(19) United States
(12) Patent Application Publication
(10) Pub. No.: US 2021/0361603 A1
(43) Pub. Date: Nov. 25, 2021

(54) METHODS OF TREATING CANCERS HAVING A DEREGULATED NRF2/KEAP1 PATHWAY

(71) Applicants: New York University, New York, NY (US); Massachusetts Institute Of Technology, Cambridge, MA (US)

(72) Inventors: Thales PAPAGIANNAKOPOULOS, New York, NY (US); Tyler JACKS, Cambridge, MA (US); Rodrigo ROMERO, Cambridge, MA (US)

(73) Assignees: New York University, New York, NY (US); Massachusetts Institute Of Technology, Cambridge, MA (US)

(21) Appl. No.: 16/483,835

(22) PCT Filed: Feb. 6, 2018
(86) PCT No.: PCT/US2018/017068

§ 371 Date: Aug. 6, 2019

Related U.S. Application Data
(60) Provisional application No. 62/454,897, filed on Feb. 6, 2017.

(51) Int. Cl. A61K 31/198; A61K 31/4184; A61K 31/282; A61K 31/675; A61K 31/655; A61K 31/519; A61K 31/495; A61K 33/243; A61K 31/5377; A61K 31/475; A61K 31/506; A61K 38/50; A61K 31/4745; A61K 38/20; A61K 38/21; A61K 38/19; A61P 35/04

(52) U.S. Cl. CPC A61K 31/198; A61P 35/04; A61K 31/282; A61K 31/675; A61K 31/655; A61K 31/519; A61K 31/495; A61K 33/243; A61K 31/5377; A61K 31/475; A61K 31/506; A61K 38/50; A61K 31/4745; A61K 38/20; A61K 38/21; A61K 38/191; A61K 38/193; A61K 31/4184

(57) ABSTRACT

The present disclosure relates to a method of treating a subject having cancer that includes selecting a subject having cancer associated with a deregulated NRF2/KEAP1 pathway and administering to the selected subject one or more inhibitors comprising a glutamine transporter inhibitor; a GPD2 inhibitor; or combination(s) thereof.

### GOVERNMENT SUPPORT

This invention was made with government support under grant number K22CA201088-01 awarded by the National Institutes of Health.

### FIELD OF THE INVENTION

The present invention relates to methods of treating cancers having a deregulated NRF2/KEAP1 pathway.

### BACKGROUND OF THE INVENTION

Lung cancer is the leading cause of cancer-related deaths in the United States and worldwide (Herbst et al., “Lung cancer,” The New England Journal of Medicine 359: 1367-1380 (2008)). A major subtype of lung cancer is non-small-cell lung cancer (NSCLC), which accounts for ˜85% of all lung cancer cases. NSCLC can be further sub-divided into three main histological subtypes: lung adenocarcinoma (LUAD), squamous cell carcinoma (SQCC) and large-cell lung cancer. Cancer genome sequencing efforts in lung adenocarcinoma (Network, “Comprehensive Molecular Profiling of Lung Adenocarcinoma,” Nature (2014)) and SQCC (Cancer Genome Atlas Research, “Comprehensive Genomic Characterization of Squamous Cell Lung Cancers,” Nature 489:519-525 (2012)) have revealed that ˜30% of human NSCLC tumors acquire gain-of-function (GOF) mutations in anti-oxidant transcription factor, nuclear factor, erythroid 2-like 2 (NFE2L2; hereafter referred to as NRF2) or loss-of-function (LOF) mutations in kelch-like ECH-associated protein 1 (KEAP1), the negative regulator of NRF2. KRAS-driven lung adenocarcinomas, a subtype of NSCLC, contain ˜20% KEAP1 mutations. The high mutation frequency of the NRF2/KEAP1 pathway suggests an important role for oxidative stress homeostasis in maintaining cancer cell survival during lung carcinogenesis. This presents an opportunity to identify novel therapeutic strategies for patients with this genetic subtype (NRF2/KEAP1 mutant) of NSCLC.

Tumorigenesis requires that cancer cells increase their metabolic output to support tumor growth. Increased metabolic activity increases oxidative stress in the form of reactive oxygen species (ROS), which can lead to oxidative damage of macromolecules. The role of ROS in the development and progression of cancer has been the subject of considerable study and debate. Initial studies suggested that ROS promotes the development and progression of cancer and therefore anti-oxidants can be used for the treatment of cancer. These studies led to clinical trials where lung cancer patients receiving anti-oxidants exhibited worse outcomes than control patients, which was unexpected. There is now emerging evidence supporting the idea that cancers increase antioxidant capacity as a stress response mechanism, suggesting that high ROS levels may constitute a barrier to tumorigenesis.

Despite advances in the field, there remains a great need to identify therapeutic strategies for the treatment of cancer, particularly NSCLC.

The present invention is directed to overcoming these and other deficiencies in the art.

### SUMMARY OF THE INVENTION

One aspect of the present invention is directed to a method of treating a subject having cancer that includes selecting a subject having cancer associated with a deregulated NRF2/KEAP1 pathway and administering to the selected subject one or more inhibitors comprising a glutamine transporter inhibitor; a GPD2 inhibitor; or combination(s) thereof.

Genetically engineered mouse models (GEMMs) of lung cancer have assisted in pre-clinical studies of human lung cancer. These GEMMs faithfully mimic human lung tumors in their progression, showing similarities both at the molecular and histopathological levels1. A model of Kras-driven lung cancer GEMM exists where lung tumors are induced in Kras^{LSL-G12D/+} (K) or Kras^{LSL-G12D/+}; p53^{flox/flox}(KP) mice after intratracheal administration of viral vectors expressing Cre-recombinase (DuPage et al. 2009, Nat Protoc 4:1064-1072), which activates a oncogenic KrasG12D allele with or without concomitant deletion of the tumor suppressor p53 in the murine lung epithelium. Recently, a novel CRISPR/Cas9-based in vivo genome engineering method was developed that allows rapid, efficient and systematic interrogation of the functional role of genes in this Kras-driven GEMM of NSCLC (Sanchez-Rivera et al. 2014, Nature 516:428-431). Using novel CRISPR/Cas9-based genome-engineering technologies, a unique and rapid research program has been established to functionally characterize lung cancer mutations and identify genotype-specific vulnerabilities that can lead to novel therapeutic approaches (Sayin et al. 2017, Cancer letters, 387:10-17). Using this approach, it has been demonstrated that CRISPR/Cas9-based somatic loss of the anti-oxidant transcription factor Nrf2 represses lung tumorigenesis, whereas loss of the negative regulator of Nrf2, Keap1, strongly promotes NSCLC progression. The GEMM data supports the tumor suppressive role of Keap1 in lung cancer. To further validate the findings from the GEMM, publically available data was analyzed for KRAS mutant lung adenocarcinomas from The Cancer Genome Atlas (TCGA). It was observed that patients with KRAS and KEAP1 mutant tumors have significantly less median survival than patients with KRAS mutant and KEAP1 wild-type tumors. To identify genotype specific vulnerabilities in Keap1 mutant Kras-driven NSCLC, a focused genetic CRISPR-based screen was performed and it was identified that loss of Gpd2 or SLC1A5 is synthetic lethal in Keap1 mutant but not wild-type GEMM-derived lung cancer cell lines. The findings of the studies described herein will justify the development of specific inhibitors for these molecules. See also Romero et al. (2017) Nature Medicine 23:1362-1368; the entire content of which is incorporated herein in its entirety.

Overall, these studies provide a platform for future characterization of lung cancer mutations in order to identify genotype-specific vulnerabilities than can lead to novel therapeutic approaches.

The invention provides a method of treating a subject having cancer, the method comprising: selecting a subject having cancer associated with a deregulated NRF2/KEAP1 pathway; and administering to the selected subject one or more inhibitors comprising a glutamine transporter inhibitor; a GPD2 inhibitor; or combination(s) thereof.

The invention provides a method of treating cancer associated with a deregulated NRF2/KEAP1 pathway in a subject, comprising administering to the subject one or more inhibitors comprising a glutamine transporter inhibitor; a GPD2 inhibitor; or combination(s) thereof. In an aspect, the cancer associated with a deregulated NRF2/KEAP1 pathway in a subject is determined by isolating a sample of cancer cells or tumor from a subject and assessing the NRF2/KEAP1 pathway in the cells or tumor from the subject. In an aspect, the cancer is lung cancer, In an aspect the cancer is NSCLC.

The invention provides a method for treating lung cancer in a subject wherein the NRF2/KEAP1 pathway is deregulated in the lung cancer or cells thereof, comprising administering to the subject one or more inhibitors comprising a glutamine transporter inhibitor; a GPD2 inhibitor; or combination(s) thereof. In an aspect the lung cancer is NSCLC.

In an aspect of the method(s) herein, the glutamine transporter inhibitor inhibits SLC1A5, SLC1A4, SLC6A19, SLC38A3, SLC38A5, SLC38A7, SLC7A5, SLC7A6, SLC7A8, SLC38A1, SLC38A2, or combinations thereof. In a particular aspect, the glutamine transporter inhibitor inhibits SLC1A5.

The method includes an aspect wherein said selecting comprises detecting deregulation of the NRF2/KEAP1 pathway in a biological sample from the subject.

In accordance with the method, the cancer is or may be selected from bladder cancer, bone marrow cancer, breast cancer, cancer of the central nervous system, cervical cancer, colon cancer, endometrial cancer, cancer of the gastric system, head and neck cancer, kidney cancer, liver cancer, lung cancer, muscle cancer, ovarian cancer, pancreatic cancer, prostate cancer, skin cancer, or thyroid cancer. In a particular aspect the cancer is lung cancer. In an aspect, the lung cancer is non-small cell lung cancer (NSCLC).

In an aspect of the methods provided herein, the cancer is mediated by a KRAS gene mutation.

In a further aspect of the invention, the one or more inhibitors are selected from the group consisting of an antibody or binding portion thereof, a nucleic acid aptamer, a peptide inhibitor, a small molecule, and combinations thereof.

In an aspect of the instant methods, the one or more inhibitors comprises the glutamine transporter inhibitor and the GPD2 inhibitor. In an aspect of the instant method, the one or more inhibitors comprises a glutamine transporter inhibitor and a GPD2 inhibitor. In an aspect, the one or more inhibitors comprises the glutamine transporter inhibitor. In an aspect, the one or more inhibitors comprises a glutamine transporter inhibitor.

In another aspect of the methods, the glutamine transporter inhibitor is GPNA. In another aspect of the method, a glutamine transporter inhibitor is GPNA.

In a further aspect of the instant methods, the one or more inhibitors comprises the GPD2 inhibitor. In a further aspect of the instant methods, a one or more inhibitor comprises a GPD2 inhibitor. In a further aspect, the GPD2 inhibitor is iGP-1, iGP-5, or a combination thereof. In another aspect, the GPD2 inhibitor is selected from iGP-1, iGP-5, or a combination thereof. In another aspect, a GPD2 inhibitor is selected from iGP-1, iGP-5, or a combination thereof.

In a further general aspect of the invention, it has been recognized that direct competition for metabolic resources represents a relevant mode of immune evasion by malignant cells in cancer, particularly in lung cancer and LUAD. Thus, tumors wherein the NRF2/KEAP pathway is deregulated, such as wherein the NRF2 pathway is upregulated or in a loss-of-function mutation in KEAP, dampen immune responses through competing for nutrients with the infiltrating immune cells. The experiments herein show that CTL activated in presence of supernatant from KPK (Krasp53Keap mutant) cells vs. KP (Krasp53 mutant) isogenic cells responded differently to stimuli. CTL activated in KPK conditioned media failed to upregulate markers of activation (such as CD25) and produced significantly less IFNγ or the effector protease, granzyme-B, compared to CD8+ T cells grown in media from KP tumor cells. The results provided herein indicate that tumor-intrinsic mutations in Keap1, for example in LUAD, impact T cell activation, proliferation and function.

The data provided herein reveals that genotype-specific changes in the NRF2 pathway of cancer cells can influence the metabolic landscape of the tumor microenvironment. In particular, changes in glutamine and serine uptake by lung cancer cells results in significant changes in T cell recruitment to the tumor, and in cytotoxic T cell activation and proliferation.

Thus, in accordance with the present invention, in addition to or in combination with modulating glutamine metabolism, such as via glutamine transporter inhibition or GPD2 inhibition, therapeutics or therapeutic approaches to enhance immune responses, block negative feedback signaling to immune cells are warranted for treatment of cancer, particularly lung cancer, particularly cancer wherein the NRF2/KEAP pathway is deregulated.

In an aspect, the invention provides a method of treating cancer in a subject, particularly lung cancer, having a deregulated NRF2/KEAP pathway, comprising administering to the subject one or more inhibitors comprising a glutamine transporter inhibitor, a GPD2 inhibitor, or combination(s) thereof, and one or more immune check-point inhibitor.

In an aspect, the invention provides a method of treating cancer in a subject, particularly lung cancer, having a deregulated NRF2/KEAP pathway, comprising administering to the subject one or more inhibitors comprising a glutamine transporter inhibitor, a GPD2 inhibitor, or combination(s) thereof, and one or more immune check-point inhibitor, immune modulator, or combination(s) thereof.

An aspect of the present invention is directed to a method of treating a subject having cancer that includes selecting a subject having cancer associated with a deregulated NRF2/KEAP1 pathway and administering to the selected subject one or more inhibitors comprising a glutamine transporter inhibitor, a GPD2 inhibitor, or combination(s) thereof, and one or more immune check-point inhibitor, immune modulator, or combination(s) thereof.

In an aspect of the invention and the method(s) hereof, checkpoint inhibitor therapy is combined with one or more glutamine transporter inhibitor, a GPD2 inhibitor, or combination(s) thereof. In an aspect, checkpoint inhibitor therapy is combined with one or more glutaminase inhibitor, such as CB839. In a further aspect checkpoint inhibitor therapy is combined with suppression of metabolic enzymes/pathways, particularly with regard to glutamine and/or serine, for enhancement of anti-tumor immunity.

In an aspect an immune check-point inhibitor may be one or more therapeutic that blocks or inhibits the immune checkpoint molecule CTLA-4, such as CTLA-4 blocking antibodies. Agents that interfere with the interactions between programmed cell death 1 protein (PD-1, CD279) and its ligand PD-1 ligand (PD-L1, CD274). Examples include PD-1 antibody, such as Nivolumab, pembrolizumab, antibody BGB-A317. PD-L1 inhibitors may include anti-PD-L1 antibodies. Examples include atezolizumab, avelumab, durvalumab.

In an aspect of the method(s) herein, one or more inhibitor is further combined with one or more of an immune modulator, including an interleukin, interferon, tumor necrosis factor (TNF) or other growth factor, or colony stimulating factor. An immune modulator may be an adjuvant. Applicable immune modulators include IDO, TDO (Platten M (2012) Cancer Research 72(21):5435-40), α-galactosyl ceramide and analogs thereof such as threitolceramide (ThrCer) and ThrCer 6, TLR ligands such as poly I:C (TLR3), MPL (TLR4), imiquimod (TLR7), R848 (TLR8) or CpG (TLR9), iCOS, OX40 and OX40 ligand, Lag3, GITR, GITR ligand interleukins, tumor necrosis factor (TNF) or other growth factors, colony stimulating factors, T cell modulators including modulators of CD8⁺ T cells, cytokines or hormones which stimulate the immune response or reduction or elimination of cancer cells or tumors (Mellman I (2011) Nature (480):480-489). Additional immune modulators include small molecules, antagonist antibodies or agonist antibodies targeting the applicable immune modulators including IDO, TDO, Toll like receptor family or iCOS, CTLA-4, PD1, PD1 ligand, OX40 and OX40 ligand, interleukins, tumor necrosis factor (TNF) or other growth factors, colony stimulating factors, T cell modulators including modulators of CD8⁺ T cells, cytokines which stimulate the immune response or reduction or elimination of cancer cells or tumors.

In accordance with the methods hereof, in one aspect the subject is a human. In accordance with the methods hereof, in one aspect the subject is a rodent. In accordance with the methods hereof, in one aspect the subject is a mammal. In accordance with the methods hereof, in one aspect the subject is a non-human mammal.

In an aspect of the method(s) hereof, said administering one or more inhibitors occurs in combination with administration of at least one other cancer therapeutic to the selected subject.

In an additional aspect, said administering occurs in combination with administration of a chemotherapeutic to the selected subject. In a particular aspect, the chemotherapeutic is selected from the group consisting of alkylating agents, antimetabolites, anthracyclines, antitumor antibiotics, platinum-based chemotherapeutics, and plant alkaloids. In another aspect, the chemotherapeutic is selected from the group consisting of oxaliplatin, cyclophosphamide, ifosfamide, thiotepa, melphalan, busulfan, nimustine, ranimustine, dacarbazine, procarbazine, temozolomide, cisplatin, carboplatin, nedaplatin, methotrexate, pemetrexed, fluorouracil, tegaful/uracil, doxifluridine, tegaful/gimeracil/oteracil, capecitabine, cytarabine, enocitabine, gemcitabine, 6-mercaptopurine, fuludarabin, pentostatin, cladribine, hydroxyurea, doxorubicin, epirubicin, daunorubicin, idarubicine, pirarubicin, mitoxantrone, amurubicin, actinomycin D, bleomycine, pepleomycin, mytomycin C, aclarubicin, zinostatin, vincristine, vindesine, vinblastine, vinorelbine, paclitaxel, docetaxel, irinotecan, irinotecan active metabolite (SN-38), nogitecan (topotecan), etoposide, prednisolone, dexamethasone, tamoxifen, toremifene, medroxyprogesterone, anastrozole, exemestane, letrozole, rituximab, imatinib, gefitinib, gemtuzumab ozogamicin, bortezomib, erlotinib, cetuximab, bevacizumab, sunitinib, sorafenib, dasatinib, panitumumab, asparaginase, tretinoin, arsenic trioxide, salts thereof, active metabolites thereof, and combinations thereof.

In another aspect of the method(s) hereof, said administering occurs in combination with administering radiation therapy to the selected subject.

In another aspect of the method(s) hereof, said administering occurs in combination with administering to the selected subject surgery, thermoablation, focused ultrasound therapy, cryotherapy, or combinations thereof.

In a further aspect of the method(s) hereof, said administering occurs in combination with administering a glutaminase inhibitor to the selected subject.

In another aspect of the method(s) hereof, administering a glutaminase inhibitor occurs in combination with administration of at least one other cancer therapeutic to the selected subject. In another aspect of the method(s) hereof, administering a glutaminase inhibitor occurs in combination with administration of at least one other chemotherapeutic to the selected subject. In another aspect of the method(s) hereof, administering a glutaminase inhibitor occurs in combination with administering radiation therapy to the selected subject.

In an aspect of the method(s), said administration of one or more inhibitors comprising a glutamine transporter inhibitor; a GPD2 inhibitor; or combination(s) thereof occurs simultaneously with administration of the at least one other cancer therapeutic.

In one aspect, said administration occurs before administration of the at least one other cancer therapeutic. In another aspect, said administration occurs after administration of the at least one other cancer therapeutic.

In a further aspect, said administration improves the efficacy of the other cancer therapeutic as compared to when said administration does not occur.

In an aspect of the method(s) hereof, the deregulated NRF2/KEAP1 pathway results in hyperactive NRF2 signaling.

In an aspect of the method(s) hereof, assessing Nqo1 levels is used to detect the deregulated NRF2/KEAP1 pathway. In a particular aspect, the Nqo1 levels are assessed using immunohistochemical staining.

In an aspect of the method(s) hereof, the NRF2/KEAP1 pathway is deregulated by a loss-of-function mutation in KEAP1, a gain-of-function mutation in NRF2, loss of KEAP1 expression by copy number loss, loss of KEAP1 expression by promoter hypermethylation, increased NRF2 expression by copy number gain, or increased NRF2 expression by promoter hypomethylation.

In accordance with any of the method(s) hereof, administering may be carried out orally, parenterally, periadventitially, subcutaneously, intravenously, intramuscularly, intraarticularly, intraperitoneally, by inhalation, by intranasal instillation, by implantation, by intracavitary or intravesical instillation, intraocularly, intraarterially, intralesionally, transdermally, or by application to mucous membranes.

In an aspect of the method(s) hereof, the method suppresses tumor growth in the subject or selected subject. In an aspect of the method(s) hereof, the method inhibits metastasis of a tumor in the subject or selected subject.

In an aspect of the method(s) hereof, the method improves or lengthens survival in the subject or selected subject. In an aspect of the method(s) hereof, the method improves or lengthens survival in the subject or selected subject versus cancer subjects, particularly cancer subjects having cancer associated with a deregulated NRF2/KEAP1 pathway, that are not administered one or more glutamine transporter inhibitor, GPD2 inhibitor, or combinations thereof.

Other objects and advantages will become apparent to those skilled in the art from a review of the following description which proceeds with reference to the following illustrative drawings.

### BRIEF DESCRIPTION OF THE DRAWINGS

FIG. 1A shows micro-computed tomography (micro-CT) quantification of total tumor volume (mm³) of tumors from sgKeap1.4 (n=5) or sgTom (n=3) infected animals at 4 and 5 months post infection. *P<0.05. Obtained from two-sided Student's t-test. All error bars denote s.e.m. FIG. 1B shows the combined quantification of tumor burden (total tumor area/total lung area) in Kras^{LSL-G12D/+}; p53^{fl/fl} animals after infection with pSECC lentiviruses. The left panel shows tumor burden 21 weeks post infection of animals infected with control sgTom (n=3) or sgKeap1.2 (n=7). The right panel shows tumor burden 21 weeks post infection of animals infected with control sgTom (n=6) or sgKeap1.4 (n=5). The asterisks indicate statistical significance obtained from comparing Kras^{LSL-G12D/+}; p53^{fl/fl}-sgKeap1 samples to Kras^{LSL-G12D/+}; p53^{fl/fl}-sgTom samples using two-sided Student's t-test, *P<0.05. All error bars denote s.e.m. FIG. 1C shows the distribution of tumor grades in Kras^{LSL-G12D/+}; p53^{fl/fl} animals 21 weeks after infection with pSECC lentiviruses expressing: control (sgTom, Kras^{LSL-G12D/+}; p53^{fl/fl} (n=7 mice)), sgKeap1.2 (Kras^{LSL-G12D/+}; p53^{fl/fl} (n=3 mice)), 1, grade 1; 2, grade 2; 3, grade 3; 4, grade 4. ***P<0.001, ****P<0.0001, obtained from two-sided Student's t-test. All error bars denote s.e.m. FIG. 1D shows quantification of phospho-Histone H3 (pHH3) positive tumor cells (pHH3 positive nuclei per mm²) to assess the mitotic index of tumor cells from lung tumors in Kras^{LSL-G12D/+}; p53^{fl/fl} animals 21 weeks after infection with pSECC lentiviruses expressing: control (sgTom, n=14 tumors), or sgKeap1.2 (n=50 tumors). *P<0.05, obtained from two-sided Student's t-test. All error bars denote s.e.m. FIG. 1E shows contingency tables demonstrating correlation between nuclear Nrf2 expression and Nqo1 expression. The top panel shows quantified tumors obtained from control sgTom infected mice. The bottom panel shows quantified tumors obtained from sgKeap1.2 infected mice (two-sided Fisher's exact test, ****P<0.0001). FIG. 1F shows representative haematoxylin and eosin (H&E) and immunohistochemistry (IHC) staining of serial sections from lung tumors of mice 21 weeks after infection with sgTom-pSECC (top panel) or sgKeap1.2-pSECC (bottom panel). Nuclear Nrf2 stain is shown in middle panels. Nqo1 stain is shown in right panels. Note the accumulation of Nrf2 and Nqo1 occurs only in tumors from sgKeap1.2-pSECC mice. Inset represents higher magnification. Size bars are 100 um. FIG. 1G is a LOF/WT pie chart. FIG. 1H shows the spread of sgKeap1.2 mutation calls.

FIG. 2A is a schematic representation of Kras^{LSL-G12D/+}; p53^{fl/fl} mice intratracheally infected with pSECC lentiviruses containing sgKeap1 or control sgTom. Mouse tumor burden was tracked by micro-computed tomography (micro-CT) at 4 and 5 months post infection. Mouse lungs were harvested 21 weeks post infection. Whole lungs were subjected to immunohistochemistry (IHC) and tumors were micro-dissected for sequencing, IHC, and generation of tumor derived cell lines. FIG. 2B shows the distribution of tumor grades in Kras^{LSL-G12D/+}; p53^{fl/fl} animals 21 weeks after infection with pSECC lentiviruses expressing: control (sgTom, Kras^{LSL-G12D/+}; p53^{fl/fl} (n=6)), sgKeap1.4 (Kras^{LSL-G12D/+}; p53^{fl/fl} (n=5)), 1, grade 1; 2, grade 2; 3, grade 3; 4, grade 4. *P<0.05, **P<0.01, ***P<0.001 obtained from two-sided Student's t-test. All error bars denote s.e.m. FIG. 2C shows the percentage of total mutation reads in tumors obtained from Kras^{LSL-G12D/+}; p53^{fl/fl} mice 21 weeks after infection with pSECC lentiviruses expressing sgKeap1.2. FIG. 2D shows the percentage of total mutation reads in tumors obtained from Kras^{LSL-G12D/+}; p53^{fl/fl} mice 21 weeks after infection with pSECC lentiviruses expressing sgKeap1.4. FIG. 2E shows representative alleles obtained from Kras^{LSL-G12D/+}; p53^{fl/fl} tumor, 22T3 or in FIG. 2F, the related metastasis 22LN, 21 weeks post infection with pSECC lentiviruses expressing sgKeap1.2. The left is a pie chart that contains the fraction of alleles over total mutant reads from tumor 22T3 or 22LN. All mutant reads falling below 1% sequencing reads are marked as other. The right panel shows Keap1 wild-type locus containing the sgKeap1.2 cut site (black) and PAM sequence (green) with the predicted cut site highlighted by a red arrow. Sequences were obtained using MiSEQ. Note that alleles A and B from FIG. 2E are found enriched in the metastatic tumor 22LN, obtained from the mediastinal lymph node indicating the ability to track genetic bottlenecks in the metastatic cascade using CRISPR and next generation sequencing as shown in FIG. 2G.

FIG. 3A is a schematic representation of the generation of the indicated cells (n=2/genotype). Parental KP cells were electroporated with pX458 containing sgTom, sgNrf2.3, or sgKeap1.4 sgRNAs and sorted for GFP as single cells. FIG. 3B shows a western blot nuclear and cytoplasmic fractionation analysis of indicated cells. HH3 and Hsp90 were used as a loading control for the nuclear fraction cytoplasmic fractions respectively. It is noted that accumulation of nuclear Nrf2 and increases in cytoplasmic Gclc occurs only in KPK clones. FIG. 3C is a heatmap representing the z-scores (z=(X−μ)/σ, where z is the z-score) of the IC₅₀ values for each indicated cells treated with the indicated compounds. Grey squares depict samples in which IC₅₀ values could not be determined. Z-score color scale depicted below the heatmap. FIG. 3D, top panel, shows glutathione (GSH) concentrations (μM) overtime after treating with 100 μM BSO (0 hr). Concentrations of GSH were normalized to the 0 hr BSO samples of each individual cell line. The bottom panel shows cell viability at same time points after addition of 100 μM BSO (0 hr). Cell viability was obtained by cell-titer glo analysis. All samples were normalized to their respective vehicle treated control (relative luminescent units). All error bars depict s.e.m. (n=3 treatments per time point/cell line). FIG. 3E shows the amount of ROS in CRISPR targeted cells as judged by fluorescence-activated cell sorting (FACS) analyses of CM-DCF fluorescence (n=3 per cell line) ****P<0.0001 obtained from 1-way Anova with Tukeys post hoc test. FIG. 3F shows oxidative stress index as judged by % 8-oxo-dG positive nuclei (n=10 per genotype) ****P<0.0001 obtained from two-sided Student's t-test. FIG. 3G shows final subcutaneous tumor masses. KP control or KPK cell lines were injected subcutaneously into the flanks of immunodeficient mice and weighed at end point. ***P<0.001, obtained from two-sided Student's t-test. FIG. 3H shows orthotopic growth measurements of KP and KPK cells (n=4). Quantitation of luminescence (photon flux) in mice orthotopically transplanted with KP or KPK cells transduced with a vector expressing Luciferase. Relative photon flux calculated by normalizing all time points per animal to initial measurements at 14 days post transplantation. ***P<0.001 obtained from 2-way Anova.

FIG. 4A, left panel, shows the Keap1 locus containing the sgKeap1.4 cut site (black) and PAM sequence (green) with the predicted cut site highlighted by a red arrow. KPK1 and KPK2 alleles screened by next generation sequencing. The right panel shows the Nrf2 locus containing the sgNrf2.3 cut site (black) and PAM sequence (green) with the predicted cut site highlighted by a red arrow. KPN1 and KPN2 alleles screened by next generation sequencing.

FIG. 4B shows real-time quantitative PCR of Nrf2 target genes, Nqo1, Hmox1, and Gclc in the indicated cell lines. The Y-axis depicts the fold change relative to KP1 for each respective target gene. Errors bars depict standard deviation (n=3 per cell line per gene). *P<0.05, **P<0.01, ***P<0.001, ****P<0.0001. Obtained from two-sided Student's t-test. Statistics are derived via the comparison of the KP1 sample for each target gene. FIG. 4C shows western blot analysis of CRISPR targeted KP clones with or without SFN (10 uM for 6 hr). GAPDH was used as a loading control. Note, accumulation of Nrf2 occurs only in the KP samples, but is not further stabilized in either KPK samples. FIGS. 4D-4F show real-time quantitative PCR of Nrf2 target genes, Nqo1, Hmox1, and Gclc in KP cell lines treated with the Nrf2 activator, SFN (10 uM for 6 hours). The Y-axis depicts the fold change relative to the KP1-SFN treated sample. Error bars depict standard deviation (n=3 per cell line per gene). *P<0.05, **P<0.01, ***P<0.001, ****P<0.0001. Obtained from two-sided Student's t-test. All statistics are derived via the comparison of the KP1-SFN sample for each target gene. FIGS. 4G-4I show dose response curves of BSO, AUR, ERA, and FIG. 4J show dose response curves of dimethyl fumarate (DMF, Nrf2 activator). Relative viability was obtained using cell-titer glo assay after 72 hours of drug treatment. Relative viability for DMF treated samples were obtained using Hoechst staining to count individually stained nuclei after 72 hours of drug treatment. All values were normalized to their respective vehicle treated control. The X-axis depicts respective drug concentration (uM) in log scale. Error bars depict s.e.m. (n=3 per cell line per treatment).

FIG. 5A shows a representative schematic of the lentivirus containing the HA tagged GOF-Nrf2 cDNA driven by a TRE-promoter. FIG. 5B shows a western blot depicting two independent KPN cell lines expressing a doxycycline inducible HA-tagged GOF-Nrf2 cDNA (KPN-ix). The GOF-Nrf2 cDNA was induced for 72 hours using doxycycline. Induced cells were treated with SFN (10 uM) for 6 hours before harvesting. The first panel shows blotting for HA. The second panel depicts an Nrf2 antibody blot. Note * depicts the expected wild-type Nrf2 size (not seen in KPN clones) and ** depicts the lower molecular weight GOF mutant lacking exon 2. The third panel depicts Gclc blotting. The final panel shows the GAPDH loading control. Note the GOF-Nrf2 cDNA is only expressed following doxycycline administration, which leads to increases in cytoplasmic Gclc. FIGS. 5C-5D show dose response curves of KPN-ix cell lines treated with BSO and 72 hours of dox induction. KPN-ix—dox is shown in dark red, and KPN-ix+dox is shown in light red. Relative viability was obtained using CellTiter-Glo® assay after 72 hours of drug treatment. All values were normalized to their respective vehicle treated control. The X-axis depicts respective BSO concentrations in log scale. Error bars depict s.e.m. (n=3 per cell line per treatment). FIG. 5E shows an IC₅₀ bar graph of KPN-ix cell lines treated with BSO and 72 hours of dox induction. KPN-ix—dox is shown in dark red, and KPN-ix+dox is shown in light red. Note, induction of the GOF-Nrf2 cDNA results in an increase IC₅₀. FIGS. 5F-5H show real-time quantitative PCR of Nrf2 target genes, Nqo1, Gclc, and Slc7all in KPN-ix (− dox dark red; + dox light red). The GOF-Nrf2 cDNA was induced for 72 hours using doxycycline. Induced cells were treated with SFN (10 uM) for 6 hours before harvesting. The Y-axis depicts the fold change relative to untreated KPN1-ix for each respective target gene. Errors bars depict standard deviation (n=3 per cell line per gene). *P<0.05, **P<0.01, ***P<0.001, ****P<0.0001. Obtained from two-sided Student's t-test. FIG. 51 shows basal reduced glutathione (GSH): oxidized glutathione (GSSG) ratios of KP (grey shades), KPN (red shades), and KPK (blue shades) cell lines (n=3 per cell line). **P<0.01, ****P<0.0001. Obtained from two-sided Student's t-test. FIG. 5J shows antioxidant (Trolox and NAC) rescue of oxidative stressed (BSO, AUR, ERA or Control) treated KPN cell lines (n=3 per cell line per antioxidant per oxidative stress condition). ****P<0.0001, obtained from 1-way Anova with Tukeys post hoc test. FIG. 5K shows the amounts of ROS in human lung cancer cell lines as judged by FACS analyses of CM-DCF fluorescence (n=3 per cell line). ****P<0.0001. Obtained from 1-way Anova with Tukeys post hoc test. FIG. 5L shows subcutaneous tumor volumes (mm³; (a²*b)*(7t/6) where a is the smaller dimension and b is the larger dimension) measured over time for 22 days. FIG. 5M shows quantification of Ki67 in tumors from mice orthotopically transplanted with KP and KPK cells (n=10). P>0.0001, Obtained from two-sided Student's t-test. FIG. 5N shows representative images from Ki67 stained KP and KPK orthotopic lung tumors. FIG. 5O shows cumulative population doublings in vitro of indicated cell lines (n=4).

FIG. 6A shows empirical cumulative distribution function (CDF) plot showing correlation of individual tumors with the NRF2 core target signature across various clinical stages within the TCGA LUAD cohort. Each curve in the plot represents a unique clinical stage as depicted in the figure legend. Clinical stage IV tumors (n=24) are highly correlated with the NRF2 core target signature and are significantly different compared to lower stage I tumors (n=251; p=0.028) KW=Kruskal-Wallis test; KS=Kolmogorov-Smirnov test. FIG. 6B shows Kaplan-Meier (KM) survival curves comparing LUAD TCGA patients stratified by their correlation with the NRF2 core target signature. TCGA LUAD tumors were binned according to their correlation with the NRF2 signature. The top 15% (n=68) correlated tumors display significantly decreased survival compared to the rest (n=389) of the TCGA LUAD cohort (p=0.009, log-rank test). FIG. 6C shows box-plot highlighting the KEAP1 wild-type (n=380) versus KEAP1 mutant (n=79) gene expression signature detected within the TCGA LUAD cohort. The Y-axis shows a significantly different signature profile between the two sets of tumors (p<2.22e-16, Mann-Whitney-Wilcoxon test). FIG. 6D shows GSEA enrichment plot showing that the NRF2 core target signature is highly enriched in the KEAP1 mutant signature derived from the TCGA LUAD cohort (FDR=0.0). FIG. 6E shows GSEA enrichment plot of the published NFE2L2.V2 signature (Malhotra et al. 2010, Nucleic Acids Res 38:5718-5734, doi:10.1093/nar/gkq212) exhibiting enrichment in the TCGA KEAP1 mutant signature (FDR=0.0). FIG. 6F shows Kaplan-Meier (KM) survival curves comparing TCGA LUAD patients stratified by their correlation with the KEAP1 mutant signature derived from TCGA patient expression profiles. The top 20% correlated patients (n=91) display decreased survival compared to the rest (n=367) of the TCGA LUAD cohort (p=0.012, log-rank test). FIG. 6G shows an empirical cumulative distribution function (CDF) plot showing correlation of individual tumors with the KEAP1 mutant signature across various clinical stages within the TCGA LUAD cohort. Each curve represents a unique clinical stage as depicted in the figure legend. Clinical stage IV tumors (n=24) are highly correlated with the KEAP1 mutant signature and are significantly different compared to stage I tumors (n=251; p=0.038, Kolmogorov-Smirnov test; KW=Kruskal-Wallis test across all stages). FIG. 6H shows an empirical cumulative distribution function (CDF) plot showing correlation of individual tumors with the KEAP1 mutant signature across various tumor grades from the TCGA LUAD cohort. Each curve represents a unique tumor grade as depicted in the figure legend. Grade III/IV tumors (n=60) exhibit significantly higher correlation with the KEAP-mutant signature compared to grade I tumors (n=146; p=0.02, Kolmogorov-Smirnov test; p_{g}=Kruskal-Wallis test across all grades).

FIG. 7A shows a pooled sgRNA library screen. The figure inlet shows a schematic of experiment. Cells were passaged 14 times before collection. Bars represent score for median sgRNA. Full representation is shown in FIG. 8A. FIG. 7B shows a western blot analysis of Slc1a5 in KP and KPK cells post selection infected with sgTom or sgSlc1a5. Hsp90 was used as loading control. FIG. 7C shows cumulative population doublings of KP and KPK cells after transduction with sgTom or sgSlc1a5 (n=4). The picture inlet shows a colony formation assay in KP and KPK cells transduced with sgTom or sgSlc1a5. ****P<0.0001 obtained from 2-way Anova. FIG. 7D shows cumulative population doublings of KRAS mutant human lung cancer cell lines either KEAP1 wt (H647 and H2009) or KEAP1 mutant (A549 and H2030) after selection with sgTom or sgSLC1A5 (n=4). ****P<0.0001 obtained from 2-way Anova. FIG. 7E shows crystal violet stain of KP and KPK cells treated with 1 mM GPNA or Vehicle for 72 hours. FIG. 7F shows cumulative population doublings of KP and KPK cells cultured in 2.0 mM or 0.5 mM Glutamine (n=4). ****P<0.0001 obtained from 2-way Anova FIG. 7G shows glutamine consumption in KP and KPK cells measured (n=3). All samples were normalized to their respective vehicle treated control. **P<0.01 obtained from 1-way Anova with Tukey's post hoc test All error bars depict s.e.m.

FIG. 8A shows a full representation of pooled sgRNA library screen related to FIG. 7A. FIG. 8B shows crystal violet stain of KP and KPK cells after transduction with sgTom, sgSlc1a5.1 or sgSlc1a5.2.cultured for 72 h. FIG. 8C shows the relative viability assayed with CellTiter-Glo® (relative luminescent units) on KP and KPK cells after treatment with GPNA for 72 hours. FIG. 8D shows crystal violet stain of KP and KPK cells cultured with 2 mM, 1 mM or 0.5 mM Glutamine cultured for 72 h. FIG. 8E shows glucose consumption (left Y-axis) and lactate excretion (right Y-axis) in KP and KPK cells measured (n=3). All samples were normalized to their respective vehicle treated control. **P<0.01 obtained from 1-way Anova with Tukey's post hoc test. All error bars depict s.e.m. FIG. 8F shows trypan blue exclusion viability counts of KP and KPK cells cultured in the presence or absence of 5 mM 2DG for 72 h. ****P<0.0001. Obtained from two-sided Student's t-test. FIG. 8G shows crystal violet stain of KP and KPK cells cultured with or without 5 mM 2DG for 72 h. All error bars depict s.e.m.

FIG. 9A shows the relative viability assayed with CellTiter-Glo® (relative luminescent units) on KP and KPK cells after treatment with CB-839 (left) or BPTES (right) for 72 hours. All data points are relative to vehicle treated controls (n=4/data point). FIG. 9B shows crystal violet stain of KP and KPK cells receiving CB-839 (250 nM), BPTES (5 uM) or control for 72 hours. FIG. 9C shows viability counts of human lung cancer cell lines that are KEAP1 wt, KEAP1 mutant or NRF2 mutant, treated with 500 nM CB-839 plotted as % of control (Individual lines in FIG. 10B) for 72 hours. **P<0.01 obtained from Mann-Whitney test. FIG. 9D shows viability counts of KP-ix cells containing a doxycycline inducible Nrf2 Aexon2 (GOF Nrf2 cDNA) treated with control, doxycycline, CB-839 (250 nM), or both doxycycline and CB-839 for 72 hours. FIG. 9E shows a schematic of Xenograft CB-839 experiments. FIG. 9F shows the response index to CB-839 Subcutaneous transplant (n=6). FIG. 9G shows the response index to CB-839 Orthotopic transplant (n=4).

FIG. 10A shows cumulative population doublings of KP and KPK cells in the presence of Vehicle, CB-839 or BPTES (n=4) after 6 days in culture. FIG. 10B shows trypan blue exclusion viability counts of indicated human lung cancer cell lines. Each cell line was cultured in the presence of vehicle control or 500 nM CB-839 (n=4). Displayed results are normalized against vehicle control treated cell lines after 72 h of treatment. FIG. 10C shows a western blot depicting two independent KP cell lines expressing a doxycycline inducible HA-tagged GOF-Nrf2 cDNA (KP-ix). The GOF-Nrf2 cDNA was induced for 72 hours using doxycycline. Induced cells were treated with SFN (10 uM) for 6 hours before harvesting. The first panel shows blotting for HA. The second panel depicts an Nrf2 antibody blot. Note * depicts the expected wild-type Nrf2 size and ** depicts the lower molecular weight GOF mutant lacking exon 2. The third panel depicts Gclc blotting. The final panel shows GAPDH loading control. Note the GOF-Nrf2 cDNA is only expressed following doxycycline administration.

FIG. 10D shows real-time quantitative PCR of Nrf2 target genes, Nqo1, Gclc, and Slc7all in KP-ix. The GOF-Nrf2 cDNA was induced for 72 hours using doxycycline. Induced cells were treated with SFN (10 uM) for 6 hours before harvesting. The Y-axis depicts the fold change relative to untreated KP1-ix for each respective target gene (n=3). FIG. 10E shows subcutaneous tumor volumes of KP and KPK treated with Vehicle or CB-839 starting from day 13 (arrow indicating treatment start) measured over time for 25 days (n=6). Related to FIG. 9F.

FIG. 10F shows final tumor masses related to FIG. 10F. *P<0.05 ****P<0.0001 obtained from 1-way Anova with Tukey's post hoc test. FIG. 10G shows orthotopic growth measurements of KP and KPK cells treated with Vehicle or CB-839 starting from day 13 (arrow indicating treatment start) (n=4). Quantitation of luminescence (photon flux) in mice orthotopically transplanted with KP or KPK cells transduced with a vector expressing Luciferase. Relative photon flux calculated by normalizing all time points per animal to initial measurements at 10 days post transplantation. Related to FIG. 9G. ***P<0.001 obtained from 2-way Anova. All error bars depict s.e.m.

FIGS. 11A-11B show loss of Keap1 accelerates lung tumorigenesis in KP animals. FIG. 11A shows temporal microCT measurements of Tumor volume in animals infected with KP (control) or KPK (sgKeap1) pSECC lentivirus. FIG. 11B shows histopathological assessment of tumor grades.

FIGS. 12A-12B show survival of patients based on KEAP1 mutation status. FIG. 12A shows Kaplan-Meier survival of patients with KRAS mutant tumors with wild-type (WT, red) or mutant (Mut, blue) KEAP1. Logrank Test p-value=0.00584. FIG. 12B shows cumulative distribution function (CDF) plots comparing the expression of 108 NRF2 targets in TCGA lung adenocarcinoma from different clinical stages. Test p-value<0.05.

FIG. 13 shows assessment of Keap1 and Nrf2 status in CRISPR/cas9 edited isogenic clones. Western blot analysis for Nrf2 and Keap1 and control GAPDH in control (KP) and CRISPR/Cas9 induced LOF of Keap1 (KPK) cells is shown. Cells were treated with oxidative stress inducer DMF.

FIGS. 14A-14B show CRISPR/cas9 genetic screen in isogenic KP and KPK clones. FIG. 14A is a schematic of depletion screen. FIG. 14B shows validation of screen candidate Slc1a5 in two independent KP and KPK clones. Synthetic lethality of Slc1a5 (sgSlca15) loss is seen in KPK and not KP cells.

FIG. 15A shows KPK cells are sensitive to serine. FIG. 15B shows KPK cells are sensitive to glutamine deprivation.

FIG. 16A shows HUSEC enables for CRISPR/cas9 somatic constitutive expression of one sgRNA and inducible expression of another. FIG. 16B shows HOTCG enables simultaneous expression of Cas9 and inducible expression of an sgRNA.

FIGS. 17A-17B show CRISPR/cas9 genetic screen in isogenic KP and KPK clones. FIG. 17A is a Schematic of depletion screen. FIG. 17B shows validation of screen candidate Gpd2 in two independent KP and KPK clones. Synthetic lethality of Gpd2 (sgGpd2) loss is seen in KPK and not KP cells.

FIG. 18 shows analysis of sensitivity using a chemical inhibitor of Gpd2, iGP-5, in KPK cells as compared to KP cells.

FIG. 19A shows HUSEC enables for CRISPR/cas9 somatic constitutive expression of one sgRNA and inducible expression of another. FIG. 19B shows HOTCG enables simultaneous expression of Cas9 and inducible expression of an sgRNA.

FIG. 20 shows a schematic of the experimental workflow for future SLC1A5 studies.

FIG. 21 shows a schematic of the experimental workflow for future GPD2 studies.

FIG. 22 shows that loss of Keap1 accelerates lung tumorigenesis and alters tumor metabolism. FIG. 22a provides micro-CT quantification of total tumor volume (mm3) of tumors from sgKeap1.4 (n=5) or sgTom (n=3) infected animals at 4 and 5 months post infection. FIG. 22b shows representative hematoxylin and eosin (H&E) and immunohistochemistry (IHC) staining of serial sections from lung tumors of mice 21 weeks after infection with sgTom-pSECC (top panel) or sgKeap1.2-pSECC (bottom panel). Inset represents higher magnification. Size bars are 100 μm. FIG. 22c shows colony formation assay in KP and KPK cells transduced with sgTom or sgSlc1a5 demonstrates synthetic lethal interaction of Slc1a5 in KPK cells. Relative consumption of glutamine (FIG. 22d) and serine (FIG. 22e) in conditioned media from a 24 hr KP or KPK tumor cell line culture. FIG. 22f provides response index to CB839 Orthotopic transplants (n=4). FIG. 22f shows model of immune cell competition for metabolites with tumor cells.

FIG. 23 shows that conditioned media from Keap1 mutant tumors results in blunting of T cell proliferation and activation. FIG. 23 a provides a schematic of in vitro T cell assays. FIG. 23b and FIG. 23c depicts naïve CD8 T cells isolated from spleens of C57BL/6 mice via negative selection using B220, Gr1, CD11b, Ter119, NK1.1, CD25, CD4 antibodies. T cells were activated in tumor conditioned media for 72 hours following α-CD3/CD28 stimulation CD8 T cells activated in Keap1 mutant (KPK) cell conditioned media displayed reduced production of granzyme B (FIG. 23b) and significantly reduced expression of activation marker CD25 (FIG. 23c) relative to T cells activated in the presence of KP tumor media. Conditioned media was derived by harvesting media from a 24 hr KP or KPK tumor cell line culture, controlled for tumor cell number.

FIG. 24 shows that K-Ras driven lung adenocarcinoma tumors with Keap1 mutation are characterized by decreased CD3 infiltration. FIG. 24a depicts analysis of immune infiltrate in Keap1 mutant tumors revealed significantly reduced immune response, characterized by absence of significant T cell infiltration. Plotted is the IC(=immune cell) scoring evaluation of individual tumors where 0=no T cell infiltration, 1=presence of individual infiltrating cells, 2=infiltrating cells with some T cell clusters and 3=infiltrating cells as well as T cell clusters found throughout the tumor periphery. Keap1 mutant tumors have a markedly lower IC score. FIG. 24b) Murine Keap1 deficient tumors are characterized by decreased CD3+ T cell infiltrate. To exclude that differences in CD3 infiltration are due to differences in tumor size we categorized individual tumors into three size categories. FIG. 24 c) Multi-color Immunofluorescent staining of a human lung adenocarcinoma with markers for: T cell CD3 (green), cytotoxic T cell CD8 (red), regulatory T cell Foxp3 (pink), inhibitory receptor PD1 (yellow), epithelial marker (brown) and nuclei (blue).

FIG. 25 depicts a novel genetically-engineered mouse model for studying immune checkpoint inhibition in lung adenocarcinoma. FIG. 25a provides experimental outline of in vivo assays. FIG. 25b describes in vitro assays. FIG. 25c shows a schematic of GOF CRISPRa screen to identify novel metabolic mediators of immune evasion.

FIG. 26 shows novel genetically-engineered mouse model for studying immune checkpoint inhibition in lung adenocarcinoma FIG. 26a provides a schematic depicting CRISPR synergistic activation mediated (SAM) system. A modified sgRNA recruits catalytically dead Cas9 that is fused to VP64 to promoter regions up stream of the transcription start site of target genes and recruits additional transcriptional activators to induced endogenous transcription of target genes. FIG. 26b shows that SAM system enables induction of endogenous PDL1 (sgPDL1) on the surface of KPK cells as compared to control (sgCtrl). FIG. 26c shows engineered KP and KPK cells expressing luciferase or luciferase fused to the model CD8 antigen OVA (LUCOS) expressing cell surface PDL1 or control were orthotopically transplanted in the lungs of C57B6/J animals and tumor growth was monitored by noninvasive luminescent imaging. Engineered expression of PDL1 enabled engraftment of cells expressing OVA. FIG. 26d provides a model of pre-clinical studies to assess the impact of suppressing tumor metabolism to enhance checkpoint inhibitor efficacy.

### DETAILED DESCRIPTION OF THE INVENTION

One aspect of the present invention is directed to a method of treating a subject having cancer that includes selecting a subject having cancer associated with a deregulated NRF2/KEAP1 pathway and administering to the selected subject one or more inhibitors comprising a glutamine transporter inhibitor; a glycerol-3-phosphate dehydrogenase 2 (GPD2) inhibitor; or combination(s) thereof.

The anti-oxidant transcription factor nuclear factor (erythroid-derived 2)-like 2 (NRF2) acts as the primary line of cellular defense against ROS to maintain oxidative homeostasis by regulating the expression of a plethora of genes involved in ROS clearance and rewiring of cellular metabolism. KEAP1, a ubiquitin ligase adaptor protein, is a negative regulator of NRF2, the key transcription factor that activates the cellular antioxidant response. Deregulation of the NRF2/KEAP1 pathway, NRF2 signaling, an increase in the intracellular concentration of glutathione, or mutations in KEAP1 or NRF2 (for example, a loss-of-function mutation in KEAP1, or a gain-of-function mutation in NRF2) confer a dependence of tumors on reduced glutathione, the major endogenous antioxidant comprised of glycine, cysteine, and glutamine-derived glutamate.

Nuclear factor (erythroid-derived 2)-like 2, also known as NRF2, is a transcription factor that in humans is encoded by the NFE2L2 gene. NRF2 is ubiquitously expressed at low levels in all human organs. The NRF2 antioxidant response pathway is the primary cellular defense against the cytotoxic effects of oxidative stress. Among other effects, NRF2 increases the expression of several antioxidant enzymes. As NRF2 regulates a major cellular defense mechanism, tight regulation is crucial to maintain cellular homeostasis. Activation of this pathway is important in preventing human diseases, such as cancer, neurodegenerative disease, cardiovascular diseases, ischemia, diabetes, pulmonary fibrosis, and inflammatory diseases. Conversely, high constitutive levels of NRF2 occur in many tumors or cancer cell lines. Moreover, overexpression of NRF2 in cancer cells protects them from the cytotoxic effects of anticancer therapies, resulting in chemo- and/or radioresistance.

Under normal or unstressed conditions, NRF2 is kept in the cytoplasm by a cluster of proteins that degrade it quickly. Under oxidative stress, NRF2 is not degraded, but instead travels to the nucleus where it binds to a DNA promoter and initiates transcription of antioxidative genes and their proteins. NRF2 is kept in the cytoplasm by Kelch like-ECH-associated protein 1 (KEAP1) and Cullin 3 which degrade NRF2 by ubiquitination. Cullin 3 ubiquitinates its substrate, NRF2. KEAP1 is a substrate adaptor, which helps Cullin 3 ubiquitinate NRF2. When NRF2 is ubiquitinated, it is transported to the proteasome, where it is degraded and its components recycled. Under normal conditions NRF2 has a half-life of only 20 minutes. Oxidative stress or electrophilic stress disrupts critical cysteine residues in KEAP1, disrupting the KEAP1-Cul3 ubiquitination system. When NRF2 is not ubiquitinated, it builds up in the cytoplasm, and translocates into the nucleus. In the nucleus, it combines (forms a heterodimer) with a small Maf protein and binds to the Antioxidant Response Element (ARE) in the upstream promoter region of many antioxidative genes, and initiates their transcription.

The NRF2/KEAP1 pathway is the major regulator of cytoprotective responses to endogenous and exogenous stresses caused by reactive oxygen species (ROS) and electrophiles. The key signaling proteins within the pathway are the transcription factor NRF2 that binds together with small Maf proteins to the antioxidant response element (ARE) in the regulatory regions of target genes, and KEAP1, the repressor protein that binds to NRF2 and promotes its degradation by the ubiquitin proteasome pathway. KEAP1 is a very cysteine-rich protein, with mouse KEAP1 having a total of 25 and human KEAP having 27 cysteine residues, most of which can be modified in vitro by different oxidants and electrophiles. Three of these residues, C151, C273 and C288, have been shown to play a functional role by altering the conformation of KEAP1 leading to nuclear translocation of NRF2 and subsequent target gene expression.

As described herein, it has been recognized that alterations to the NRF2/KEAP1 pathway sensitizes cancer cells to glutamine transporter inhibition and/or GPD2 inhibition.

As used herein, a “glutamine transporter inhibitor” may be any inhibitor that inhibits or reduces glutamine transport into cells.

Glutamine transport processes are guaranteed by a number of membrane transporters which share specificity for glutamine but show differences in transport modes. The pleiotropic role of glutamine may be the reason why glutamine transporters, belonging to several protein families, are redundant and ubiquitous. In particular, the SLC (solute carrier)-type transporters (˜400 in number) in mammalian cells consist of 52 distinct gene families, grouped solely based on the amino acid sequence (primary structure) of the transporter proteins and not on their transport function. Among them are the transporters for amino acids. Fourteen of them, capable of transporting glutamine across the plasma membrane, are found in four families: SLC1, SLC6, SLC7, and SLC38. Some of the glutamine transporters are obligatory exchangers whereas some function as active transporters in one direction. While most glutamine transporters mediate the influx of the amino acid into cells, some actually mediate the efflux of the amino acid out of the cells. Glutamine transporters play important roles in a variety of tissues, including the liver, brain, kidney, and placenta.

Emerging evidence implicates oncogenic signaling pathways with nutrient uptake in cancer cells. The neutral amino acid glutamine is essential for cell growth and proliferation. In addition to glucose, cancer cells utilize glutamine as a carbon source for ATP production and biosynthesis.

In one embodiment, the glutamine transporter inhibitor inhibits SLC1A5, SLC1A4, SLC6A19, SLC38A3, SLC38A5, SLC38A7, SLC7A5, SLC7A6, SLC7A8, SLC38A1, SLC38A2, or combinations thereof. In certain embodiments, the glutamine transporter inhibitor inhibits SLC1A5.

The SLC1A5 transporter is a transporter of neutral amino acids such as glutamine and a range of other neutral amino acids (such as glutamine, leucine, and isoleucine) in and out of a cell in a Na⁺ dependent, obligate amino acid exchange process. These transporters, by shuttling of various amino acids across the cell membrane, may facilitate or regulate various physiological processes such as cell growth, proliferation, or even glutamatergic neurotransmission via the glutamate/glutamine cycle. SLC1A5 expression is associated with oncogenic MYC and KRAS, and may be relevant in clinically important tumors, including lung, colon, and pancreas associated with MYC and KRAS.

GPD2, also known as glycerol-3-phosphate dehydrogenase 2, localizes to the inner mitochondrial membrane and catalyzes the conversion of glycerol-3-phosphate to dihydroxyacetone phosphate, using FAD as a cofactor. Along with GPD1, the encoded protein constitutes the glycerol phosphate shuttle, which reoxidizes NADH formed during glycolysis.

The terms “treat”, “treating”, “treatment” and the like are used interchangeably herein and mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed the disease, or reducing the severity of a disease, recurrence of a disease, sustaining remission from a disease, or lengthening survival of those afflicted with a disease. “Treating” as used herein covers treating a disease in a vertebrate and particularly a mammal and most particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease. The term ‘treating’ or ‘treatment’ of a disease, in one embodiment, to ameliorating the disease or growth or progression of diseased cells (i.e., arresting the disease or growth of cancerous or pre-cancerous cells or tumors or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or growth or progression of diseased cells, either physically, (e.g., stabilization of a discernible symptom), physiologically, (e.g., stabilization of a physical parameter), or both. In a further embodiment, ‘treating’ or ‘treatment’ relates to slowing the progression of a disease or reducing recurrence or sustaining remission of a disease.

As used herein, “subject” refers to any animal that is amenable to treatment in accordance with the methods of the present invention. The subject may be, in one embodiment, a mammal. Exemplary mammalian subjects include, without limitation, humans, non-human primates, dogs, cats, rodents (e.g., mouse, rat, guinea pig), horses, cattle and cows, sheep, and pigs.

In one embodiment, selecting a subject having cancer associated with a deregulated NRF2/KEAP1 pathway comprises detecting deregulation of the NRF2/KEAP1 pathway in a biological sample from the subject.

The NRF2/KEAP1 pathway may be altered in one of several different mechanisms. In one embodiment, the NRF2/KEAP1 pathway in said subject is deregulated and the deregulated NRF2/KEAP1 pathway results in hyperactive NRF2 signaling. As described below, NRF2 signaling could be hyperactive due to several different factors, such as disruption of KEAP1-NRF2 binding or a reduction or loss in the function of KEAP1, caused, for example, by any of the mechanisms described below. NRF2 signaling could also be hyperactive due to factors that affect expression of NRF2, including, but not limited to, induction of NRF2 by oncogenic signaling or mutations in NRF2 that result in increasing its expression or function, for example including as described below.

Hyperactive NRF2 signaling may also result when the NRF2/KEAP1 pathway in said subject is deregulated and ubiquitination of NRF2, which ordinarily ultimately leads to its degradation, is prevented. The prevention, blocking or reduction of ubiquitinization of NRF2 results in hyperactive NRF2 signaling. The NRF2/KEAP1 pathway may be deregulated due to a mutation in either NRF2 or KEAP1. The mutation may be any mutation that disrupts the function of KEAP1 or a mutation in either KEAP1 or NRF2 that prevents the binding of KEAP1 to NRF2. In one embodiment, the mutation is a somatic mutation.

In another embodiment, hyperactive NRF2 signaling may result when the NRF2/KEAP1 pathway is deregulated due to epigenetic silencing of KEAP1 expression. Epigenetic silencing of KEAP1 expression results in constitutively activated NRF2 signaling. For example, DNA hypermethylation at the promoter region of KEAP1 may result in epigenetic silencing of KEAP1 expression.

In yet another embodiment, hyperactive NRF2 signaling may result due to accumulation of disruptor proteins leading to dissociation of the KEAP1-NRF2 complex. Examples of disruptor proteins include, but are not limited to, cyclin-dependent kinase inhibitor p21, and polyubiquitin binding protein p62.

Transcriptional induction of NRF2 is another mechanism by which the NRF2/KEAP1 pathway may be deregulated and NRF2 signaling would be hyperactive. The transcriptional induction of NRF2 may, for example, be caused by oncogenic signaling due to elements including, but not limited to, K-Ras, B-Raf and c-Myc.

In another embodiment, hyperactive NRF2 signaling may also result due to post-translational modification of KEAP1 that affects binding of KEAP1 to NRF2. Any of the amino acid residues of KEAP1 may undergo post-translational modification. In one embodiment, the cysteine residues of KEAP1 are modified. Post-translational modification of KEAP1, for example modification of the cysteine residues, includes, but is not limited to, succination of critical cysteine residues. Critical KEAP1 cysteine residues include, but are not limited to, those at positions 151, 273 and 288. KEAP1 human sequence (e.g. Uniprot KB Q14145) and mouse KEAP1 sequence (e.g. Uniprot KB Q9Z2X8) are known and publicly available, among other mammalian KEAP1 sequences.

In other embodiments, the NRF2/KEAP1 pathway is deregulated by a loss-of-function mutation in KEAP1, a gain-of-function mutation in NRF2, loss of KEAP1 expression by copy number loss, loss of KEAP1 expression by promoter hypermethylation, increased NRF2 expression by copy number gain, or increased NRF2 expression by promoter hypomethylation.

Methods of detecting deregulation of the NRF2/KEAP1 pathway in a biological sample are well known in the art. The present disclosure is not limited to particular types of detection methods. Examples include, but are not limited to, screening cells for gene expression at the mRNA or protein level (e.g., via reporter genes in live cells or molecular analysis), protein analysis (e.g., immunoassays such as e.g., Western blot and mass spectrometry analysis), epigenome analysis (e.g., methylation status of genes and/or promoters), copy number variation (CNV) assays, and screening for mutations or polymorphisms (e.g., SNPs).

In some embodiments, gene expression or other protein analysis is performed using immunoassays, mass spectrometry, PCR, and/or Northern blot, which are all well known in the art. Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

By way of example, one may use an artificially constructed expression construct containing the Nrf2 promoter element and an artificial reporter gene. Alternatively, one may use PCR or Northern blotting to determine expression levels of Nrf2 mRNA, or Western blotting to determine Nrf2 protein levels. Exemplary procedures for determining expression levels of Nrf2 are described in Kwak et al., Mol. Cell. Biol. 2002, 22(9):2883-2892 and Kwak et al., Mol. Med., 2001, 7:135-145, each of which are hereby incorporated by reference in its entirety.

In some embodiments, subcellular localization and/or nuclear translocation of Nrf2 may be performed using assays that include cell staining, or analysis of cytoplasmic versus nuclear cell extracts. For example, a Nrf2-green fluorescence protein (GFP) fusion protein construct can be made and introduced into cells and visualized as described in, e.g., Kraft et al., J. Neurosci., 2004, 24, 1101-1112; and Satoh et al., PNAS, 2006, 103(3):768-773, each of which is hereby incorporated by reference in its entirety.

Expression levels and/or activity of one or more genes under the control of Nrf2 can be assessed using endogenous or artificially introduced reporter genes in reporter constructs introduced into cells. For example, expression levels of endogenous or exogenously introduced NQO1 may be determined. Alternatively, a reporter gene construct with one or more ARE sites operably linked to a reporter gene (e.g., luciferase or GFP) can be made, as described in, e.g., Satoh et al., PNAS, 2006, 103(3):768-773, which is hereby incorporated by reference in its entirety. Expression levels of an Nrf-2 induced gene product can be measured at the protein (e.g., by Western blotting or enzymatic activity assays) or at the mRNA levels (e.g., by PCR). Methods for performing RT-PCT are described in, e.g., Calabrese et al., J. Neurosci. Res., 2005, 79:509-521, which is hereby incorporated by reference in its entirety, for HO-1, in Wierinckx et al., J. Neuroimmunology, 2005, 166:132-143, which is hereby incorporated by reference in its entirety, for NQO1. Methods for measuring enzymatic activity of NQO1, using for example, menadione as a substrate, are described in Dinkova-Kostova et al., PNAS, 2001, 98:3404-09, which is hereby incorporated by reference in its entirety, or by Prochaska et al., Anal. Biochem., 1988, 169:328-336, which is hereby incorporated by reference in its entirety.

In certain embodiment, the stability of Nrf2/Keap1 complexes may be measured. Such assays may include analysis of immunoprecipitated complexes with Nrf2 and/or Keap1 or other Nrf2/Keap1-associated proteins as described in, e.g., Satoh et al., PNAS, 2006, 103(3):768-773, which is hereby incorporated by reference in its entirety. Anti-Keap1 antibodies can be produced using methods known in the art and are available commercially from, for example, Santa Cruz Biotechnology. Accordingly, in some embodiments, the Nrf-2 pathway is activated so that the stability of Nrf2/Keap1 complexes is increased by, for example, at least 30%, 50%, 100%, 200%, 500% or more as compared to the non-activated state.

A variety of nucleic acid sequencing methods are also contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Different kinds of biological assays may include microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., those described in table 1) by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

In one embodiment, single sample gene set enrichment (ssGSEA) can be used as described in U.S. Patent Application Publication No. 20160058759 to Heffernan, which is hereby incorporated by reference in its entirety) to score NRF2 activity in lung cancer cell lines with expression data published as part of the Cancer Cell Line Encyclopedia (CCLE). ssGSEA assigns an enrichment based on the distribution of a gene set relative all other genes within a single sample (Barbie et al, 2009 Nature 462:108-112, which is hereby incorporated by reference in its entirety). The ssGSEA enrichment scores may be median centered and normalized by the standard deviation of the enrichment scores from all lung cell lines in the CCLE panel. The NRF2 signature gene set may be based on a known set of NRF2 target genes: ABCC1, ABCC2, G6PD, GCLC, GCLM, GRK6, GSR, GSTM4, HMOX1, ME1, MGST1, NQO1, PRDX1, TXN and TXNRD 1.

Southern and Northern blotting may be used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

Nucleic acids may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The methylation levels of non-amplified or amplified nucleic acids can be detected by any conventional means. For example, Methylplex-Next Generation Sequencing (M-NGS) methodology may be utilized. In other embodiments, the methods described in U.S. Pat. Nos. 7,611,869, 7,553,627, 7,399,614, and/or 7,794,939, each of which is herein incorporated by reference in its entirety, are utilized. Additional detection methods include, but are not limited to, bisulfate modification followed by any number of detection methods (e.g., probe binding, sequencing, amplification, mass spectrometry, antibody binding, etc.) methylation-sensitive restriction enzymes and physical separation by methylated DNA-binding proteins or antibodies against methylated DNA (See e.g., Levenson, Expert Rev Mol Diagn. 2010 May; 10(4): 481-488; which is hereby incorporated by reference in its entirety).

The cancer may be any type of cancer. In certain embodiments, the cancer is bladder cancer, bone marrow cancer, breast cancer, cancer of the central nervous system, cervical cancer, colon cancer, endometrial cancer, cancer of the gastric system, head and neck cancer, kidney cancer, liver cancer, lung cancer, muscle cancer, ovarian cancer, pancreatic cancer, prostate cancer, skin cancer, or thyroid cancer.

Lung cancer is the leading cause of cancer-related deaths worldwide. Accordingly, in other embodiments, the cancer is lung cancer. A major subtype of lung cancer is non-small-cell lung cancer (NSCLC). NSCLC remains one of the most lethal and aggressive solid subtypes of lung cancer, and about 30% of NSCLC acquire activating mutations in NRF2 or KEAP1. Thus, in other embodiments, the cancer is non-small cell lung cancer (NSCLC).

In one embodiment, the cancer is mediated by a KRAS gene mutation.

In one embodiment, the one or more inhibitors are selected from the group consisting of an antibody or binding portion thereof, a nucleic acid aptamer, a peptide inhibitor, a small molecule, and combinations thereof.

An antibody of the present invention encompasses any immunoglobulin molecule that specifically binds to a glutamine transporter or GPD2 protein as described above. As used herein, the term “antibody” is meant to include intact immunoglobulins derived from natural sources or from recombinant sources, as well as immunoreactive portions (i.e., antigen binding portions) of intact immunoglobulins. The antibodies of the present invention may exist in a variety of forms including, for example, polyclonal antibodies, monoclonal antibodies, intracellular antibodies (“intrabodies”), antibody fragments (e.g. Fv, Fab and F(ab)2), as well as single chain antibodies (scFv), chimeric antibodies, and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc Natl Acad Sci USA 85:5879-5883 (1988); Bird et al, “Single-Chain Antigen-Binding Proteins,” Science 242:423-426 (1988), which are hereby incorporated by reference in their entirety).

Methods for monoclonal and polyclonal antibody production may be carried out using the techniques described herein or other well-known in the art (MONOCLONAL ANTIBODIES—PRODUCTION, ENGINEERING AND CLINICAL APPLICATIONS (Mary A. Ritter and Heather M. Ladyman eds., 1995), and Ed Harlow and David Lane, USING ANTIBODIES: A LABORATORY MANUAL (Cold Spring Harbor Laboratory Press, 1988), which are hereby incorporated by reference in its entirety).

For monoclonal antibody production, the process generally involves obtaining immune cells (lymphocytes) from the spleen of a mammal which has been previously immunized with the antigen of interest either in vivo or in vitro. The antibody-secreting lymphocytes are fused with myeloma cells or transformed cells, which are capable of replicating indefinitely in cell culture, thereby producing an immortal, immunoglobulin-secreting cell line. Fusion with mammalian myeloma cells or other fusion partners capable of replicating indefinitely in cell culture is achieved by standard and well-known techniques, for example, by using polyethylene glycol (PEG) or other fusing agents (Milstein and Kohler, “Derivation of Specific Antibody-Producing Tissue Culture and Tumor Lines by Cell Fusion,” Eur J Immunol 6:511 (1976), which is hereby incorporated by reference in its entirety). The immortal cell line, which may be murine, but may also be derived from cells of other mammalian species, is selected to be deficient in enzymes necessary for the utilization of certain nutrients, to be capable of rapid growth, and have good fusion capability. The resulting fused cells, or hybridomas, are cultured, and the resulting colonies screened for the production of the desired monoclonal antibodies. Colonies producing such antibodies are cloned, and grown either in vivo or in vitro to produce large quantities of antibody.

Alternatively monoclonal antibodies can be made using recombinant DNA methods (i.e. synthetic antibodies) as described in U.S. Pat. No. 4,816,567 to Cabilly et al, which is hereby incorporated by reference in its entirety. The polynucleotide(s) encoding a monoclonal antibody can further be modified using recombinant DNA technology to generate alternative antibodies. For example, the constant domains of the light and heavy chains of a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

The monoclonal antibody of the present invention can be a humanized antibody. Humanized antibodies are antibodies that contain minimal sequences from non-human (e.g., murine) antibodies within the variable regions. Such antibodies are used therapeutically to reduce antigenicity and human anti-mouse antibody responses when administered to a human subject. In practice, humanized antibodies are typically human antibodies with minimal to no non-human sequences. An antibody can be humanized by substituting the complementarity determining region (CDR) of a human antibody with that of a non-human antibody (e.g., mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

The antibody of the present invention can also be a human monoclonal antibody. A human antibody is an antibody produced by a human or an antibody having an amino acid sequence corresponding to an antibody produced by a human. Human antibodies can be produced using various techniques known in the art. Immortalized human B lymphocytes immunized in vitro or isolated from an immunized individual that produce an antibody directed against a target antigen can be generated (See e.g., Reisfeld et al., MONOCLONAL ANTIBODIES AND CANCER THERAPy 77 (Alan R. Liss ed., 1985) and U.S. Pat. No. 5,750,373 to Garrard, which are hereby incorporated by reference in their entirety). Also, the human antibody can be selected from a phage library, where that phage library expresses human antibodies (Vaughan et al., “Human Antibodies with Sub-Nanomolar Affinities Isolated from a Large Non-immunized Phage Display Library,” Nature Biotechnology, 14:309-314 (1996); Sheets et al., “Efficient Construction of a Large Nonimmune Phage Antibody Library: The Production of High-Affinity Human Single-Chain Antibodies to Protein Antigens,” Proc. Natl. Acad. Sci. U.S.A. 95:6157-6162 (1998); Hoogenboom et al., “By-passing Immunisation. Human Antibodies From Synthetic Repertoires of Germline VH Gene Segments Rearranged In Vitro,” J Mol Biol 227:381-8 (1992); Marks et al., “By-passing Immunization. Human Antibodies from V-gene Libraries Displayed on Phage,” J Mol Biol 222:581-97 (1991), which are hereby incorporated by reference in their entirety). Human antibodies can also be made in transgenic mice containing human immunoglobulin loci that are capable upon immunization of producing the full repertoire of human antibodies in the absence of endogenous immunoglobulin production. This approach is described in U.S. Pat. No. 5,545,807 to Surani et al.; U.S. Pat. No. 5,545,806 to Lonberg et al.; U.S. Pat. No. 5,569,825 to Lonberg et al.; U.S. Pat. No. 5,625,126 to Lonberg et al.; U.S. Pat. No. 5,633,425 to Lonberg et al.; and U.S. Pat. No. 5,661,016 to Lonberg et al., which are hereby incorporated by reference in their entirety.

Also suitable for use in the present invention are antibody fragments engineered to bind to intracellular proteins, i.e. intrabodies. Intrabodies are generally obtained by selecting a single variable domain from variable regions of an antibody having two variable domains (i.e., a heterodimer of a heavy chain variable domain and a light chain variable domain). Single chain Fv fragments, Fab fragments, ScFv-Cκ fusion proteins, single chain diabodies, V_{H}—C_{H}1 fragments, and even whole IgG molecules are suitable formats for intrabody development (Kontermann R. E., “Intrabodies as Therapeutic Agents,” Methods 34:163-70 (2004), which is here by incorporated by reference in its entirety).

Intrabodies having antigen specificity for a specific epitope can be obtained from phage display, yeast surface display, or ribosome surface display. Methods for producing libraries of intrabodies and isolating intrabodies of interest are further described in U.S. Published Patent Application No. 20030104402 to Zauderer and U.S. Published Patent Application No. 20050276800 to Rabbitts, which are hereby incorporated by reference in their entirety. Methods for improving the stability and affinity binding characteristics of intrabodies are described in WO2008070363 to Zhenping, and Contreras-Martinez et al., “Intracellular Ribosome Display via SecM Translation Arrest as a Selection for Antibodies with Enhanced Cytosolic Stability,” J Mol Biol 372(2):513-24 (2007), which are hereby incorporated by reference in their entirety.

By way of example, monoclonal antibodies, KM4008, KM4012, and KM4018, against SLC1A5 are described in Suzuki et al., “Establishment of Monoclonal Antibodies Against Cell Surface Domains of ASCT2/SLC1A5 and Their Inhibition of Glutamine-Dependent Tumor Cell Growth,” Biochem Biophys Res Comm pii: S0006-291X(16)31952-0. doi: 10.1016/j.bbrc. 2016.11.089 (2016), which is hereby incorporated by reference in its entirety.

Inhibitory peptides may be chemically synthesized using known peptide synthesis methodology or may be prepared and purified using recombinant technology. Such peptides are usually at least about 5 amino acids in length, but can be anywhere from 5 to 100 amino acids in length. Such peptides may be identified without undue experimentation using well known techniques. Techniques for screening peptide libraries for peptides that are capable of specifically binding to a polypeptide target, in this case a glutamine transporter or GPD2, are well known in the art (see e.g., U.S. Pat. No. 5,556,762 to Pinilla et al.; U.S. Pat. No. 5,750,373 to Garrard et al.; U.S. Pat. No. 4,708,871 to Geysen; U.S. Pat. No. 4,833,092 to Geysen; U.S. Pat. No. 5,223,409 to Ladner et al.; U.S. Pat. No. 5,403,484 to Ladner et al.; U.S. Pat. No. 5,571,689 to Heuckeroth et al.; U.S. Pat. No. 5,663,143 to Ley et al.; and PCT Publication Nos. WO84/03506 to Geysen and WO84/03564 to Geysen, which are hereby incorporated by reference in their entirety).

Nucleic acid aptamers are characterized by a single-strand and have secondary structure that may possess one or more stems (i.e., base-paired regions) as well as one or more non base-paired regions along the length of the stem. These non base-paired regions can be in the form of a bulge or loop (e.g., internal loop) along the length of the stem(s) and/or a loop at the end of the one or more stem(s) (e.g., hairpin loop). These nucleic acid aptamers possess specificity in binding to a particular target molecule, and they noncovalently bind their target molecule through an interaction such as an ion-ion force, dipole-dipole force, hydrogen bond, van der Waals force, electrostatic interaction, stacking interaction or any combination of these interactions.

Identifying suitable nucleic acid aptamers basically involves selecting aptamers that bind a particular target molecule with sufficiently high affinity (e.g., K_{d}<500 nM) and specificity from a pool or library of nucleic acids containing a random region of varying or predetermined length. For example, identifying suitable nucleic acid aptamers of the present invention can be carried out using an established in vitro selection and amplification scheme known as SELEX. The SELEX scheme is described in detail in U.S. Pat. No. 5,270,163 to Gold et al.; Ellington and Szostak, “In Vitro Selection of RNA Molecules that Bind Specific Ligands,” Nature 346:818-822 (1990); and Tuerk and Gold, “Systematic Evolution of Ligands by Exponential Enrichment: RNA Ligands to Bacteriophage T4 DNA Polymerase,” Science 249:505-510 (1990), each of which is hereby incorporated by reference in their entirety. An established template-primer system (Bartel et al., “HIV-1 Rev Regulation Involves Recognition of Non-Watson-Crick Base Pairs in Viral RNA,” Cell 67:529-536 (1991), which is hereby incorporated by reference in its entirety) can be adapted to produce RNA molecules having a stretch of about 38-40 random bases sandwiched between 5′ and 3′ constant regions.

The synthetic oligonucleotide templates can be amplified by polymerase chain reaction and then transcribed to generate the original RNA pool. Assuming that ten percent of the RNA molecules are free of chemical lesions that prevent second-strand synthesis and transcription, this pool would contain more than 3×10¹³ different sequences. Because filter binding is applicable for most protein targets, it can be used as the partitioning device, although other suitable schemes can be used. The selected primary RNA aptamers can be cloned into any conventional subcloning vector and sequenced using any variation of the dideoxy method. Next, the secondary structure of each primary RNA aptamer can be predicted by computer programs such as MulFold or mFOLD (Jaeger et al., “Improved Predictions of Secondary Structures for RNA,” Proc. Natl. Acad. Sci. USA 86:7706-7710 (1989); Zuker, “On Finding All Suboptimal Foldings of an RNA Molecule,” Science 244:48-52 (1989), each of which is hereby incorporated by reference in its entirety). Mutational studies can be conducted by preparing substitutions or deletions to map both binding sites on the RNA aptamer and its target molecule, as well as to further enhance aptamer binding affinity, as described in the accompanying Examples.

Aptamers generated from SELEX experiments can be optimized to produce second generation aptamers with improved properties (Eaton et al., “Post-SELEX Combinatorial Optimization of Aptamers,” Bioorg. Med. Chem. 5:1087-1096 (1997), which is hereby incorporated by reference in its entirety). Through successive rounds of affinity maturation of a primary SELEX clone, it is possible to obtain aptamers that possess improved affinity for their target as compared to the original clone. Therefore, as will be understood, prior to using aptamers in cell-based experiments, each aptamer can be optimized.

If any cross-reactivity is observed, then a doped library can be prepared and subjected to “negative selection,” also called “counter-SELEX.” There is considerable precedent that documents the ability of negative selection to generate aptamers with high degrees of selectivity, even among closely related molecules (Tuerk et al., “Using the SELEX Combinatorial Chemistry Process to Find High Affinity Nucleic Acid Ligands to Target Molecules,” Methods Mol Biol. 67:219-230 (1997); Rink et al., “Creation of RNA Molecules that Recognize the Oxidative Lesion 7,8-dihydro-8-hydroxy-2′-deoxyguanosine (8-oxodG) in DNA,” Proc Natl Acad Sci USA 95:11619-11624 (1998); Haller et al., “In vitro Selection of a 7-Methyl-guanosine Binding RNA that Inhibits Translation of Capped mRNA Molecules,” Proc Natl Acad Sci USA 94:8521-8526 (1997); Edwards et al., “DNA-oligonucleotide Encapsulating Liposomes as a Secondary Signal Amplification Means,” Anal Chem. 79:1806-1815 (1997), each of which is hereby incorporated by reference in its entirety). To perform negative selection, RNAs bound to receptor-agarose are subjected to a washing step in which the buffer contains other receptor molecules or peptides that replicate one or more binding domains thereof. This results in the elution of aptamers that have undesirable cross-reactivity. The RNAs that remain bound to the agarose beads are then eluted with the target of interest, and amplified as in the classic SELEX procedure. This process is can be repeated until highly selective clones are generated.

Optimization of aptamers can also be achieved during re-selection by using rigorous washing conditions in all steps, including the use of high temperature (37° C. or 45° C.) washing buffers, mild denaturants, and low salt and high salt washes, etc. The proposed stringent washing conditions are intended to select for aptamers that bind more tightly to the target, and thereby improve the over affinity. An additional benefit of generating RNA aptamers that bind with higher affinity to the target is that lower concentrations of therapeutic agents of the present invention will be needed for therapeutic in vivo applications.

Another method to use during optimization is the use of a smaller bias during doping. For example, the library can be doped with a 2:1:1:1 ratio instead of 5:1:1:1. This will result in more library members being substantially different from the parent aptamer.

The SELEX procedure can also be modified so that an entire pool of aptamers with binding affinity can be identified by selectively partitioning the pool of aptamers. This procedure is described in U.S. Patent Application Publication No. 2004/0053310 to Shi et at, which is hereby incorporated by reference in its entirety.

Single stranded DNA aptamers have advantages for in vitro settings due to their ease of synthesis and greater stability. Recent studies have argued that proper buffer conditions and certain RNA sugar modifications can lead to highly stable RNAs (Osborne et al., “Aptamers as Therapeutic and Diagnostic Reagents: Problems and Prospects,” Curr Opin Chem Biol. 1:5-9 (1997); Faria et at, “Sugar Boost: When Ribose Modifications Improve Oligonucleotide Performance,” Current Opinion in Molecular Therapeutics 10:168-175 (2008), each of which is hereby incorporated by reference in its entirety). Moreover, as part of the optimization and stabilization process, stabilizing hairpins can be added which markedly enhance aptamer levels in cells (Blind et al., “Cytoplasmic RNA Modulators of an Inside-out Signal-transduction Cascade,” Proc Natl Acad Sci USA 96:3606-3610 (1999), which is hereby incorporated by reference in its entirety). Regardless, DNA aptamer sequences that bind selectively to targets would be inexpensive to synthesize and provide additional assurance of sensor stability in solution phase or microarray-based assays.

SELEX can be performed as readily with DNA as with RNA (Breaker, “DNA Aptamers and DNA Enzymes,” Curr Opin Chem Biol. 1:26-31 (1997), which is hereby incorporated by reference in its entirety). The absence of a 2′-OH does not substantially impair the ability of DNA to fold or adopt structures. Indeed, SELEX has been used to identify DNAs that bind both small molecules and proteins, with structures that are reminiscent of RNA aptamers. Thus, DNA aptamers can be developed and subjected to analogous mutagenesis and truncation studies to identify suitable target-binding DNA molecules.

As used herein, “nucleic acid” includes both DNA and RNA, in both D and L enantiomeric forms, as well as derivatives thereof (including, but not limited to, 2′-fluoro-, 2′-amino, 2′O-methyl, 5′iodo-, and 5′-bromo-modified polynucleotides). Nucleic acids containing modified nucleotides (Kubik et al., “Isolation and Characterization of 2′fluoro-, 2′amino-, and 2′fluoro-amino-modified RNA Ligands or Human IFN-gamma that Inhibit Receptor Binding,” J. Immunol. 159:259-267 (1997); Pagratis et al., “Potent 2′-amino, and 2′-fluoro-2′-deoxy-ribonucleotide RNA Inhibitors of Keratinocyte Growth Factor,” Nat. Biotechnol. 15:68-73 (1997), each which is hereby incorporated by reference in its entirety) and the L-nucleic acids (sometimes termed Spiegelmers®), enantiomeric to natural D-nucleic acids (Klussmann et al., “Mirror-image RNA that Binds D-adenosine,” Nat. Biotechnol. 14:1112-1115 (1996) and Williams et al., “Bioactive and nuclease-resistant L-DNA Ligand of Vasopressin,” Proc. Natl. Acad. Sci. USA 94:11285-11290 (1997), each which is hereby incorporated by reference in its entirety), and non-natural bases are used to enhance biostability. In addition, the sugar-phosphate backbone can be replaced with a peptide backbone, forming a peptide nucleic acid (PNA), other natural or non-natural sugars can be used (e.g., 2′-deoxyribose sugars), or phosphothioate or phosphodithioate can be used instead of phosphodiester bonds. The use of locked nucleic acids (LNA) is also contemplated.

Small molecule inhibitors of both SLC1A5 and GPD2, which may be suitable, are available and known in the art. Small molecule inhibitors of SLC1A5 include, but are not limited to, GPNA (Esslinger et al., “N-gamma-aryl Glutamine Analogues as Probes of the ASCT2 Neutral Amino Acid Transporter Binding Site,” Bioorg Med Chem 13: 1111-1118, which is hereby incorporated by reference in its entirety); 1,2,3-dithiazoles (Oppedisano et al., “Inactivation of the Glutamine/Amino Acid Transporter ASCT2 by 1,2,3-dithiazoles: Proteoliposomes as a Tool to Gain Insights in the Molecular Mechanism of Action and of Antitumor Activity,” Toxicol Appl Pharmacol 265(1):93-102 (2012), which is hereby incorporated by reference in its entirety); benzylserine and benzylcysteine (Grewer and Grabsch, “New Inhibitors For the Neutral Amino Acid Transporter ASCT2 Reveal its Na+-Dependent Anion Leak,” J Physiol 557(Pt 3):747-59 (2004), which is hereby incorporated by reference in its entirety); 2-Amino-4-bis(aryloxybenzyl)aminobutanoic acids (Schulte et al., “2-Amino-4-bis(aryloxybenzyl)aminobutanoic Acids: a Novel Scaffold for Inhibition of ASCT2-Mediated Glutamine Transport,” Bioorg Med Chem Lett 26(3):1044-7 (2016), which is hereby incorporated by reference in its entirety); 2-Substituted N-glutamylanilides (Schulte et al., “2-Substituted N-glutamylanilides as Novel Probes of ASCT2 With Improved Potency,” Bioorg Med Chem Lett 25(1):113-6 (2015), which is hereby incorporated by reference in its entirety); and serine biphenyl-4-carboxylate (Albers et al., “Defining Substrate and Blocker Activity of Alanine-Serine-Cysteine Transporter 2 (ASCT2) Ligands with Novel Serine Analogs,” Mol Pharmacol 81(3):356-65 (2012), which is hereby incorporated by reference in its entirety)

In one embodiment, the glutamine transporter inhibitor is GPNA.

Small molecule inhibitors of GPD2 have also been described in the art and include, without limitation, i-GP1 and iGP-5 (Orr et al., “Novel Inhibitors of Mitochondrial sn-Glycerol 3-Phosphate Dehydrogenase,” PLOS One 9(2);e89938 (2014), which is hereby incorporated by reference in its entirety); and α-Tocopheryl succinate (Rauchova et al., “Inhibition of Mitochondrial Glycerol-3-phosphate Dehydrogenase by α-Tocopheryl Succinate,” Int J Biochem Cell Biol 53:409-13 (2014), which is hereby incorporated by reference in its entirety).

In one embodiment, the GPD2 inhibitor is iGP-1, iGP-5, or a combination thereof.

The inhibitors of the present invention can be administered via any standard route of administration known in the art, including, but not limited to, parenteral (e.g., intravenous, intraarterial, intramuscular, subcutaneous injection, intrathecal), oral (e.g., dietary), topical, transmucosal, or by inhalation (e.g., intrabronchial, intranasal or oral inhalation, intranasal drops). Typically, parenteral administration is the preferred mode of administration.

Therapeutic agents of the present invention are formulated in accordance with their mode of administration. For oral administration, for example, the therapeutic agents of the present invention are formulated into an inert diluent or an assimilable edible carrier, enclosed in hard or soft shell capsules, compressed into tablets, or incorporated directly into food. Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the invention in such therapeutically useful compositions is such that a suitable dosage will be obtained.

Also specifically contemplated are oral dosage forms of the agents of the invention. The agents may be chemically modified so that oral delivery of the derivative is efficacious. Generally, the chemical modification contemplated is the attachment of at least one moiety to the component molecule itself, where said moiety permits inhibition of proteolysis and uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, “Soluble Polymer-Enzyme Adducts,” In: Enzymes as Drugs, Hocenberg and Roberts, eds., Wiley-Interscience (1981), which is hereby incorporated by reference in entirety). Other polymers that could be used are poly-1,3-dioxolane and poly-1,3,6-tioxocane. Preferred for pharmaceutical usage are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; a sweetening agent such as sucrose, lactose, sucrulose, or saccharin. A dosage unit form may contain, in addition to one or more of the above materials, a liquid carrier such as a fatty oil.

The therapeutic agents of the invention may also be formulated for parenteral administration. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Effective doses of the therapeutic agents of the present invention vary depending upon many different factors, including type and stage of cancer, mode of administration, target site, physiological state of the patient, other medications or therapies administered, and physical state of the patient relative to other medical complications. Treatment dosages need to be titrated to optimize safety and efficacy.

In a preferred embodiment, the administering step is repeated periodically as needed (e.g., hourly, daily, weekly, monthly, yearly).

The inhibitors of the present invention can be administered in a single dose or multiple doses. The dosage can be determined by methods known in the art and can be dependent, for example, upon the individual's age, sensitivity, tolerance and overall well-being. Suitable dosages for antibodies can be from about 0.1 mg/kg body weight to about 10.0 mg/kg body weight per treatment.

The inhibitors of the present invention can be administered to an individual or subject (e.g., a human) alone or in conjunction with one or more other inhibitors of the invention. In one embodiment, the one or more inhibitors comprise a glutamine transporter inhibitor and a GPD2 inhibitor. In another embodiment, the one or more inhibitors comprise a glutamine transporter inhibitor. In a further embodiment, the one or more inhibitors comprise a GPD2 inhibitor.

The inhibitors of the invention can be used, alone or in combination with other pharmaceutically active compounds, to treat conditions such as those disclosed hereinabove. The inhibitors of the invention and other pharmaceutically active compound(s) can be administered simultaneously (either in the same dosage form or in separate dosage forms) or sequentially. Accordingly, in one embodiment, the invention comprises methods for treating a condition by administering to the subject a therapeutically-effective amount of one or more inhibitors of the invention and one or more additional pharmaceutically active compounds.

In one embodiment, there is provided a pharmaceutical composition comprising one or more compounds of the present invention, one or more additional pharmaceutically active compounds, and a pharmaceutically acceptable carrier. In another embodiment, the one or more additional pharmaceutically active compounds is chosen from anti-cancer drugs, anti-proliferative drugs, and anti-inflammatory drugs. In certain embodiments, the anti-cancer agent is chosen from a platinum-based agent, a taxane-based agent, an immunotherapy, and a targeted therapy. In certain embodiments, the targeted therapy is an inhibitor of MEK kinase, HSP90, CDK4, or the mTOR pathway.

The inhibitors, e.g., SLC1A5 and GPD2 inhibitors, disclosed herein are also optionally used in combination with other therapeutic reagents that are selected for their therapeutic value for the condition to be treated. In general, the inhibitors described herein and, in embodiments where combination therapy is employed, other agents do not have to be administered in the same pharmaceutical composition and, because of different physical and chemical characteristics, are optionally administered by different routes. The initial administration is generally made according to established protocols and then, based upon the observed effects, the dosage, modes of administration and times of administration subsequently modified. In certain instances, it is appropriate to administer an inhibitor compound, as disclosed herein, in combination with another therapeutic agent. By way of example only, the therapeutic effectiveness of an inhibitor is enhanced by administration of another therapeutic agent (which also includes a therapeutic regimen) that also has therapeutic benefit. Regardless of the disease, disorder or condition being treated, the overall benefit experienced by the patient is either simply additive of the two therapeutic agents or the patient experiences an enhanced (i.e., synergistic) benefit. Alternatively, if a compound disclosed herein has a side effect, it may be appropriate to administer an agent to reduce the side effect; or the therapeutic effectiveness of a compound described herein may be enhanced by administration of an adjuvant.

Therapeutically effective dosages vary when the drugs are used in treatment combinations. Methods for experimentally determining therapeutically effective dosages of drugs and other agents for use in combination treatment regimens are documented methodologies. Combination treatment further includes periodic treatments that start and stop at various times to assist with the clinical management of the patient. In any case, the multiple therapeutic agents (one of which is a glutamine transporter inhibitor and/or a GPD2 inhibitor, as disclosed herein) may be administered in any order, or simultaneously. If simultaneously, the multiple therapeutic agents are optionally provided in a single, unified form, or in multiple forms (by way of example only, either as a single pill or as two separate pills).

In some embodiments, one of the therapeutic agents is given in multiple doses, or both are given as multiple doses. If not simultaneous, the timing between the multiple doses optionally varies from more than zero weeks to less than twelve weeks.

In addition, the combination methods, compositions and formulations are not to be limited to the use of only two agents, the use of multiple therapeutic combinations are also envisioned. It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually employed varies widely, in some embodiments, and therefore may deviate from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also administered sequentially, with either agent being administered by a regimen calling for two-step administration. The two-step administration regimen optionally calls for sequential administration of the active agents or spaced-apart administration of the separate active agents. The time between the multiple administration steps ranges from a few minutes to several hours, depending upon the properties of each pharmaceutical agent, such as potency, solubility, bioavailability, plasma half-life and kinetic profile of the pharmaceutical agent.

In another embodiment, a glutamine transporter inhibitor, e.g., an SLC1A5 inhibitor, and/or a GPD2 inhibitor is optionally used in combination with procedures that provide additional benefit to the patient. A glutamine transporter inhibitor and/or a GPD2 inhibitor and any additional therapies are optionally administered before, during, or after the occurrence of a disease or condition, and the timing of administering the composition containing a glutamine transporter inhibitor, e.g., an SLC1A5 inhibitor, and/or a GPD2 inhibitor varies in some embodiments. Thus, for example, a glutamine transporter inhibitor and/or a GPD2 inhibitor is used as a prophylactic and is administered continuously to subjects with a propensity to develop conditions or diseases in order to prevent the occurrence of the disease or condition. A glutamine transporter inhibitor, e.g., an SLC1A5 inhibitor, and/or a GPD2 inhibitor and compositions are optionally administered to a subject during or as soon as possible after the onset of the symptoms. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that in some embodiments of the invention various alternatives to the embodiments described herein are employed in practicing the invention.

In one embodiment, the administering of the glutamine transporter inhibitor and/or a GPD2 inhibitor occurs in combination with administration of at least one other cancer therapeutic to the selected subject.

A glutamine transporter inhibitor, e.g., an SLC1A5 inhibitor, and/or a GPD2 inhibitor disclosed herein can be used in combination with anti-cancer drugs, including but not limited to the following classes: alkylating agents, antimetabolites, anthracyclines, antitumor antibiotics, platinum-based chemotherapeutics, and plant alkaloids.

In one embodiment, the administering occurs in combination with administration of a chemotherapeutic to the selected subject. In certain embodiments, the chemotherapeutic is selected from the group consisting of oxaliplatin, cyclophosphamide, ifosfamide, thiotepa, melphalan, busulfan, nimustine, ranimustine, dacarbazine, procarbazine, temozolomide, cisplatin, carboplatin, nedaplatin, methotrexate, pemetrexed, fluorouracil, tegaful/uracil, doxifluridine, tegaful/gimeracil/oteracil, capecitabine, cytarabine, enocitabine, gemcitabine, 6-mercaptopurine, fuludarabin, pentostatin, cladribine, hydroxyurea, doxorubicin, epirubicin, daunorubicin, idarubicine, pirarubicin, mitoxantrone, amurubicin, actinomycin D, bleomycine, pepleomycin, mytomycin C, aclarubicin, zinostatin, vincristine, vindesine, vinblastine, vinorelbine, paclitaxel, docetaxel, irinotecan, irinotecan active metabolite (SN-38), nogitecan (topotecan), etoposide, prednisolone, dexamethasone, tamoxifen, toremifene, medroxyprogesterone, anastrozole, exemestane, letrozole, rituximab, imatinib, gefitinib, gemtuzumab ozogamicin, bortezomib, erlotinib, cetuximab, bevacizumab, sunitinib, sorafenib, dasatinib, panitumumab, asparaginase, tretinoin, arsenic trioxide, salts thereof, active metabolites thereof, and combinations thereof.

In certain embodiments, methods disclosed herein further comprise administering non-chemical methods of cancer treatment. In certain embodiments, the method further comprises administering radiation therapy. In certain embodiments, the method further comprises administering surgery, thermoablation, focused ultrasound therapy, cryotherapy, or any combination thereof.

In one embodiment, the administering of the glutamine transporter inhibitor and/or a GPD2 inhibitor occurs in combination with administering a glutaminase inhibitor to the selected subject.

Glutamine metabolism, i.e., glutaminolysis is regulated by mitochondrial glutaminase (GLS), the rate limiting enzyme that catalyzes the conversion of glutamine to glutamate and ammonia. Mammalian cells contain two genes that encode glutaminase: the kidney-type (GLS-1) and liver-type (GLS-2) enzymes. Each has been detected in multiple tissue types, with GLS-1 being widely distributed throughout the body. GLS-1 is a phosphate-activated enzyme that exists in humans as two major splice variants, a long form (referred to as KGA) and a short form (GAC), which differ only in their C-terminal sequences. Both forms of GLS-1 are thought to bind to the inner membrane of the mitochondrion in mammalian cells, although at least one report suggests that glutaminase may exist in the intramembrane space, dissociated from the membrane. GLS is frequently overexpressed in human tumors and has been shown to be positively regulated by oncogenes such as Myc.

Glutaminase inhibitors have been described in the art and include, without limitation, all of those described in U.S. Patent Application Publication No. 2016/0058759 to Heffernan, which is hereby incorporated by reference in its entirety.

In one embodiment, the administration occurs simultaneously with administration of the at least one other cancer therapeutic. In another embodiment, the administration occurs before administration of the at least one other cancer therapeutic. In yet another embodiment, the administration occurs after administration of the at least one other cancer therapeutic.

In one embodiment, the administration improves the efficacy of the other cancer therapeutic as compared to when said administration does not occur.

In another embodiment, the method suppresses tumor growth in the selected subject. As used herein, “suppresses tumor growth” is meant to include a decrease of the number of tumor cells entering the cell cycle, tumor cell death or the decrease of tumor cell metastasis. In a further embodiment, the method inhibits metastasis of a tumor in the selected subject.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLES

### Example 1—Keap1 is a Tumor Suppressor with Genotype-Specific Metabolic Vulnerabilities in KRAS Driven Lung Cancer

Genetically engineered mouse models (GEMMs) of lung cancer have greatly assisted in the functional characterization of genetic variants in human lung cancers. The Kras^{LSL-G12D/+}; p53^{flox/flox} (KP) GEMMs of human LUAD faithfully mimic human KRAS-driven LUAD in their progression displaying similarities at the molecular and histopathological levels following intratracheal administration of viral vectors expressing Cre-recombinase (Carr et al., “Glutamine Uptake and Metabolism are Coordinately Regulated by ERK/MAPK During T Lymphocyte Activation,” J. Immunol. 185:1037-1044 (2010), which is hereby incorporated by reference in its entirety). pSECC, a CRISPR/Cas9-based in vivo genome engineering method that allows for rapid interrogation of genetic putative driver events that cooperate with oncogenic Kras in the KP GEMM (Sanchez-Rivera et al., “Rapid Modelling of Cooperating Genetic Events in Cancer Through Somatic Genome Editing,” Nature 516:428-431 (2014); Mazur et al., “Combined Inhibition of BET Family Proteins and Histone Deacetylases as a Potential Epigenetics-Based Therapy for Pancreatic Ductal Adenocarcinoma,” Nat. Med. 21:1163-1171 (2015); Davidson et al., “Environment Impacts the Metabolic Dependencies of Ras-Driven Non-Small Cell Lung Cancer,” Cell Metab 23:517-528 (2016), each of which is hereby incorporated by reference in its entirety), was recently developed. The requirement for cancer cells to overcome oxidative stress barriers during tumorigenesis is conserved in these GEMMs, which supports the use of these models to study the role of oxidative homeostasis during carcinogenesis (Bauer et al., “Targeted Deletion of Nrf2 Reduces Urethane-Induced Lung Tumor Development in Mice,” PLoS One 6:e26590 (2011); DeNicola et al., “Oncogene-Induced Nrf2 Transcription Promotes ROS Detoxification and Tumorigenesis,” Nature 475:106-109 (2011); Satoh et al., “Nrf2 Prevents Initiation but Accelerates Progression Through the Kras Signaling Pathway During Lung Carcinogenesis,” Cancer Res. 73:4158-4168 (2013); Sayin et al., “Antioxidants Accelerate Lung Cancer Progression in Mice,” Sci. Transl. Med. 6: 221ra215 (2014); Chio, I I et al., “NRF2 Promotes Tumor Maintenance by Modulating mRNA Translation in Pancreatic Cancer,” Cell 166:963-976 (2016); Satoh et al., “NRF2 Intensifies Host Defense Systems to Prevent Lung Carcinogenesis, but After Tumor Initiation Accelerates Malignant Cell Growth,” Cancer Res. 76:3088-3096 (2016), each of which is hereby incorporated by reference in its entirety).

### Materials and Methods

Lentiviral Vectors and sgRNA Cloning

The U6-sgRNA-EFS-Cas9-2A-Cre (pSECC; Sanchez-Rivera et al. 2014, Nature 516:428-431, doi:10.1038/nature13906) lentiviral vector was constructed by assembling four parts with overlapping DNA ends using Gibson assembly. Briefly, a 2.2 kb part (corresponding to the U6-Filler fragment from lentiCRISPR-V2 (addgene #52961), a 0.3 kb part (corresponding to the EFS promoter from lentiCRISPR-V2, a 5.3 kb part (corresponding to a Cas9-2A-Cre fragment, which was generated by assembly PCR) and a 5.7 kb lentiviral backbone were assembled using Gibson assembly following manufacturer guidelines. Detailed cloning strategies and primer sequences are available on request. For sgRNA cloning, the pSECC vector was digested with BsmBI and ligated with BsmBI-compatible annealed oligos.

Cell Culture

Cells were maintained in DMEM or RPMI supplemented with 10% Fetal Bovine Serum and gentamicin. Cell lines expressing rtTA were kept under Neomycin selection (400 ug/mL). Cell lines expressing dox inducible Nrf2 constructs remained under Hygromycin selection (600 ug/mL). Cells were treated with inhibitors D,L-Sulforaphane (SFN, EMD Millipore Calbiochem), Dimethyl fumarate (DMF, Sigma Aldrich), L-Buthionine-sulfoximine (BSO, Sigma Aldrich), Auranofin (AUR, TOCRIS bioscience), Erastin (ERA, Sigma Aldrich), L-Glutamic acid y-(p-nitroanilide)-hydrochloride (GPNA, Sigma Aldrich), 2-deoxy-D-glucose (2DG, Acros Organics), BPTES (Sigma Aldrich), CB-839 (provided by Craig J. Thomas) and antioxidants Trolox (Acros Organics) and N-acetyl-L-cysteine (NAC, Sigma Aldrich). Viability in the presence of all compounds was assessed by cell titer glo (Promega #G7570) and trypan blue exclusion on a Countess II automated cell counter (Life Technologies). For clonogenic and low-density assays, cells were stained with Crystal Violet solution (25% Methanol 75% H20). For cell counts after DMF treatment cells were fixed with 4% paraformaldehyde for 15 minutes at 4 C. Cells were then washed in ice cold PBS and then stained with Hoechst DNA stain. Plates were quantified using a Tecan infinite M200 Pro plate reader or a SpectraMax M5 Miroplate reader (Molecular Devices).

Focused CRISPR/Cas9 Genetic Screen

Oligonucleotides for sgRNAs were synthesized by Integrated DNA Technologies, annealed in vitro and inserted into lentiCRISPR-V2 (Sanjana et al. (2014, Nat Methods 11, 783-784, doi:10.1038/nmeth.3047). Gibson Assembly products were then transformed into E. coli 10G SUPREME electrocompetent cells (Lucigen). This plasmid pool was used to generate lentivirus-containing supernatants. The titer of lentiviral supernatants was determined by infecting targets cells at several amounts of virus in the presence of polybrene (8 ug/mL), counting the number of drug resistant infected cells after 3 days of selection. KP and KPK cells were infected at an MOI of ˜0.5 and selected with puromycin (3 ug/ml) 72 hours after infection. An initial pool of cells was harvested for genomic DNA extraction. The remaining cells were cultured for 14 doublings, after which cells were harvested for genomic DNA extraction. sgRNA inserts were PCR amplified, purified and sequenced on a MiSeq (Illumina) according to prior studies (Sanjana et al. (2014, Nat Methods 11, 783-784, doi:10.1038/nmeth.3047). Sequencing reads were mapped and the abundance of each sgRNA was tallied. Gene score is defined as the median log₂ fold change in the abundance between the initial and final population of all sgRNAs targeting that gene. The differential gene score is the difference between KP and KPK cell gene scores.

Immunoblotting

Cells were lysed in 250 μL ice-cold RIPA buffer (Pierce, #89900) supplemented with 1× Complete Mini inhibitor mixture (Roche, #11 836 153 001) and mixed on a rotator at 4° C. for 30 minutes. The protein concentration of the cell lysates was quantified using the Bio-Rad DC Protein Assay (Catalog #500-0114). 50-80 μg of total protein was separated on 4-12% Bis-Tris gradient gels (Bio-Rad) by SDS-PAGE and then transferred to nitrocellulose membranes. The following antibodies were used for immunoblotting: anti-FLAG (Sigma, F1804, 1:1000), anti-GAPDH (Santa Cruz, sc-25778, 1:500), anti-Hsp90 (BD, #610418, 1:10,000), anti-Nrf2 (Santa-Cruz, sc-722, 1:200 and custom antibody provided by Edward Schmidt at 1:200), anti-Keap1 (CST, #8047, 1:1000), anti-Gclc (Santa Cruz, sc-22755, 1:200) anti-S1c1a5 (Santa Cruz, ASCT2 (M-63) sc-99003, 1:100).

Immunohistochemistry and Immunofluorescence

Mice were euthanized by carbon dioxide asphyxiation. Lungs were perfused through the trachea with 4% paraformaldehyde (PFA), fixed overnight, transferred to 70% ethanol and subsequently embedded in paraffin. Sections were cut at a thickness of four micrometers and stained with H&E for pathological examination. Chromogenic immunohistochemistry (IHC) was performed on a Ventana Medical Systems Discovery XT instrument with online deparaffinization using Ventana's reagents and detection kits and antigen retrieved in Ventana Cell Conditioner 1 or 2. The following antibodies were used for IHC: anti-phospho-Histone H3 (pHH3) (Ser10; Cell Signaling, 9701, 1:200), anti-Ki67 (Spring Bioscience, Cat #M3062, 1:400), anti-Nqo1 (Sigma Aldrich, HPA007308, 1:100) anti-Nrf2 (Provided by Edward E. Schmidt lab, 1:100), anti-8-Hydroxydeoxyguanosine(8-oxo-dg) (Abcam, ab48508, N45.1, 1:200). Horseradish peroxidase (HRP) detection was used for NQO1, NRF2, pHH3 and Ki67. Alkaline phosphatase (AP) detection was used for 8-OXO and was visualized with Fast Red chromogen. NQO1 and NRF2 was antigen retrieved in Ventana Cell Conditioner 1 (Tris-Borate-EDTA). Antigen retrieval was performed with Ventana Cell Conditioner 2 (Citrate) for 8-OXO, Ki67 and pHH3. Pictures were obtained using a Nikon 80i microscope with a DS-U3 camera and MS-elements software and with a digital whole slide scanner Leica SCN400F and Slidepath software version 4.0.8.

Genomic DNA Isolation

Genomic DNA from entire snap-frozen left lung lobes or microdissected tumors was isolated using the High Pure PCR Template Preparation Kit (Roche) following manufacturer guidelines. PCR products for MiSeq were amplified using Herculase II Fusion DNA polymerase (Agilent).

Mice All animal studies described in this study were approved by the MIT Institutional Animal Care and Use Committee. Kras^{LSL-G12D} and Trp53^{flox} mice have already been described (Jackson et al. (2001, Genes Dev 15, 3243-3248, doi:10.1101/gad.943001); Jackson et al. (2005, Cancer Res 65, 10280-10288, doi:10.1158/0008-5472.CAN-05-2193)). Mice were infected intratracheally with lentiviruses as described (DuPage et al. 2009, Nat Protoc 4:1064-1072, doi:10.1038/nprot.2009.95). Total lung area occupied by tumor was measured on hematoxylin and eosin (H&E) stained slides using NIS-elements software.

Lentiviral Production

Lentiviruses were produced by co-transfection of 293T cells with lentiviral backbone constructs and packaging vectors (delta8.2 and VSV-G) using TranslT-LT1 (Mirus Bio). Supernatant was collected 48 and 72 hours post-transfection, concentrated by ultracentrifugation at 25,000 RPM for 90 minutes and resuspended in an appropriate volume of OptiMEM (Gibco).

Lentiviral vectors and sgRNA cloning pSECC lentiviral vector and cloning strategy was previously described (Sanchez-Rivera et al. 2014, Nature 516:428-431, doi:10.1038/nature13906). For CRISPR experiments the lentiCRISPR-V2 lentiviral vector was used (Shalem et al. (2014, Science 343, 84-87, doi:10.1126/science.1247005). For sgRNA cloning, the lentiCRISPR-V2 vector was digested with BsmBI and ligated with BsmBI-compatible annealed oligos for sgRNAs.

Tumor Purity Correction

Lung lobe and microdissected tumor genomic DNA was used to perform real-time PCR based analysis to detect the relative levels of the un-recombined lox-stop-lox Kras^{G12D} allele using forward primer 5′-ctcttgcctacgccaccagctc-3′ and reverse primer 5′-agctagcca ccatggcttgagtaagtctgc a-3′. To correct for DNA loading of each sample, we amplified chr5:10054507-10054621 using forward primer 5′-gaagaaattagagggcatgcttc-3′ and reverse primer 5′-cttctcccagtgaccttatgta-3′. Real-time PCR reaction was performed using KAPA Fast SYBR master mix in a Roche LightCycler Real-Time PCR instrument. To calculate percent purity we performed the following calculations for each sample: ΔCp^{tumorX}=Cp^{Chr5}−Cp^{LSL-KrasG12D} to normalize for sample loading and then calculated 1/ΔΔCp=(ΔCp^{X}−ΔCp^{LungControl}) for each sample.

Transcriptome Analysis

RNA was collected from cells as before (Papagiannakopoulos et al. (2016, Cell Metab 24, 324-331, doi:10.1016/j.cmet.2016.07.001) with RNeasy plus mini kit (Qiagen), sequenced with X analyzed with YZ. For Real Time qPCR analysis, complementary DNA (cDNA) was synthesized from RNA with the High Capacity cDNA Reverse Transcription Kit(Applied Biosystems #4368814). Genes Slc711, Gclc, Hmox1, and Ngo1 were analyzed by quantitative reverse transcription polymerase chain reaction on LightCycler 480 II (Roche). RI qPCR primers (Supplementary Sequences).

Glutaminase Inhibitor

Animals were treated as before (Davidson et al. 2016, Cell Metab 23:517-528, doi:10.1016/j.cmet.2016.01.007) with 200 mg/kg CB-839 or vehicle twice a day after tumor establishment phase. The vehicle contained 25% (w/v) hydroxypropyl-β-cyclodextrin in 10 mmol/L citrate (pH 2.0), and CB-839 was formulated at 20 mg/mL for a final dosing volume of 10 mL/kg.

Extracellular Flux Measurements

Extracellular flux measurements were calculated by extracting fresh and spent medium supernatant from tracing experiments after 24 hours of growth. Cells were assumed to grow exponentially over the culture period. Glucose, lactate and glutamine were measured using YSI biochemistry analyzer (Yellow Springs Instruments, Yellow Springs, Ohio).

ROS and Glutathione

ROS in cultured cells were measured by incubating 10⁶ cells with 5 μM CM-H2DCFDA (C6827, Life Technologies) for 30 min at 37° C. DCF fluorescence was acquired on the Attune NxT (ThermoFisher) flow cytometer and analyzed using FlowJo software (Tree Star). Reduced (GSH) and oxidized glutathione (GSSG) was measured with a GSH/GSSG-Glo Assay kit (V6611, Promega) for the indicated amount of time.

Statistics

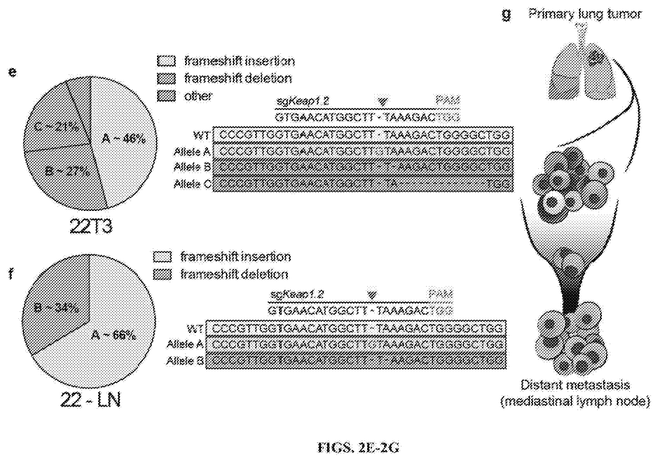

For statistical analyses, GraphPad Prism software v.6.03 was used: P-values were determined by Student's t-test for all measurements of tumor burden and IHC quantifications except for contingency tables, in which Fisher's exact test or Chi-square test were used. One-way analysis of variance (ANOVA) with Tukey's post hoc test were used for comparisons between multiple groups; for analysis between groups over multiple time measurements (growth curves) two-way ANOVA was used. Standardized IC50 values for the heatmap displayed in FIG. 3C were calculated as follows: z=(X−μ)/σ, where z is the z-score. All error bars denote s.e.m.

Bioinformatic Analysis of CRISPR-Targeted Loci

For libraries prepared with the Nextera DNA library prep kit, Illumina MiSeq reads were trimmed to 120 bp after reviewing base quality profiles, in order to drop lower quality 3′ ends. Traces of Nextera adapters were clipped using the FAS TX toolkit (Hannon Lab, CSHL) and pairs with each read greater than 15 bp in length were retained. Additionally, read pairs where either read had 50% or more bases below a base quality threshold of Q30 (Sanger) were dropped from subsequent analyses. For PCR amplicons (sequenced at the MGH sequencing facility), 142 bp paired-end reads were used in downstream analyses. The reference sequence of the target locus was supplemented with 10 bp genomic flanks and was indexed using an enhanced suffix array (Abouelhoda et al. (2004, Journal of Discrete Algorithms 2, 53-86, doi:http://dx.doi.org/10.1016/S1570-8667(03)00065-0). Read ends were anchored in the reference sequence using 10 bp terminal segments for a suffix array index lookup to search for exact matches. A sliding window of unit step size and a maximal soft-clip limit of 10 bp was used to search for possible anchors at either end of each read. For each read, optimal Smith-Waterman dynamic programming alignment (Smith et al. (1981, J Mol Biol 147, 195-197) was performed between the reduced state space of the read sequence and the corresponding reference sequence spanning the maximally distanced anchor locations. Scoring parameters were selected to allow for sensitive detection of short and long insertions and deletions while allowing for up to four mismatches and the highest scoring alignment was selected. Read pairs with both reads aligned in the proper orientation were processed to summarize the number of wild-type reads and the location and size of each insertion and deletion event. Overlapping reads within pairs were both required to support the event if they overlapped across the event location. Additionally, mutation events and wild-type reads were summarized within the extents of the sgRNA sequence and PAM site by considering read alignments that had a minimum of 20 bp overlap with this region. Mutation calls were translated to genomic coordinates and subsequently annotated using Annovar (Wang et al. (2010, Nucleic Acids Res 38, e164, doi:10.1093/nar/gkq603). The alignment and post-processing code was implemented in C++ along with library functions from SeqAn (Doring et al. (2008, BMC Bioinformatics 9, 11, doi:10.1186/1471-2105-9-11) and SSW and utility functions in Perl and R (www.R-project.org). Mutation calls were subjected to manual review using the Integrated Genomics Viewer (IGV) (Thorvaldsdottir et al. (2013, Brief Bioinform 14, 178-192, doi:10.1093/bib/bbs017).

Human Clinical Data Analyses

Genomic data for lung adenocarcinoma patient samples (n=548) were obtained from the Cancer Genome Atlas (TCGA LUAD; http://cancergenome.nih.gov/). This included RNA-seq gene expression profiles of primary tumor patient samples (n=488), mutation calls, and associated clinical data (n=458 patients with RNA-seq data for primary tumors had associated survival data). Individual sample expression profiles were scored with gene expression signatures using ssGSEA (Mootha et al. (2003, Nat Genet 34, 267-273, doi:10.1038/ng1180); Subramanian et al. (2005, Proc Natl Acad Sci USA 102, 15545-15550, doi:10.1073/pnas.0506580102). Patients were stratified according to their correlation score, into equal top and bottom percentile sets (or top-scoring n % versus rest of the cohort). Kaplan-Meier survival analysis was conducted between these sets of patients and the log-rank test was used to assess significance. The murine-derived Keap1 signature was similarly used to perform Kaplan-Meier survival analyses after translation of mouse gene names to human nomenclature (www.genenames.org). Additionally, the Cox proportional hazards regression model was used to analyze the prognostic value of the human-derived KEAP1 mutant signature across all patients within the TCGA LUAD cohort, in the context of additional clinical covariates. All univariate and multivariable analyses were conducted within a 5-year survival timeframe. The following patient and tumor-stage clinical characteristics were used: Signature (KEAP1 mutant signature strong vs. weak correlation); Gender (male vs. female); Age (years, continuous); Smoking History (reformed >15 yrs vs. non-smoker, reformed <15 yrs vs. non-smoker, current smoker vs. non-smoker); Union for International Cancer Control (UICC) TNM Stage specification (Stage III/IV vs. I/II); UICC T score specification (T2 vs. T1, T3/T4 vs. T1); UICC N score specification (N1/N2 vs. NO). Hazard ratio proportionality assumptions for the Cox regression model were validated by testing for all interactions simultaneously (p=0.723). Interactions between the KEAP1 mutant signature and TNM stage, T score, and N score (significant covariates in the model) were tested using a likelihood ratio test (LRT) to contrast a model consisting of both covariates with another model consisting of both covariates plus an interaction term. No statistically significant difference was found between the two models (TNM: p=0.445, T score: p=0.455, N score: p=0.494; likelihood ratio test). To test for statistically significant associations between the KEAP1 mutant signature correlation scores and TCGA LUAD TNM stage (stagel-IV) and grade levels (T-scores), the Kurskal-Wallis test was used to assess overall significance and the Kolmogorov-Smirnov test was used to assess pairwise differences. Results were visualized using Empirical Cumulative Distribution Function (ECDF) plots. All statistical analyses were conducted in R (www.R-project.org) and all survival analyses and were conducted using the survival package in R (Abouelhoda et al. (2004, Journal of Discrete Algorithms 2, 53-86, doi:http://dx.doi.org/10.1016/S1570-8667(03)00065-0).

Nrf2 Core Target Signature

Three published datasets were utilized to derive a high-confidence 108-gene signature of Nrf2-induced targets. Normalized microarray expression data for GSE38332 (Singh et al. (2013, J Clin Invest 123, 2921-2934, doi:10.1172/JCI66353) was downloaded from the Gene Expression Omnibus (GEO). Differential expression analysis using R/limma (Ritchie et al. 2015.+ Nucleic Acids Res 43, e47, doi:10.1093/nar/gkv007) was performed to identify genes that are differentially regulated between control and Nrf2-siRNA treated samples. A list of Nrf2-induced genes (n=433) was identified using FDR <0.05 and fold-change <1.5 thresholds. Nrf2 targets (n=345) derived by (Mitsuishi et al. 2012, Cancer Cell 22:66-79, doi:10.1016/j.ccr.2012.05.016) from microarray analysis of A549 cells treated with Nrf2 siRNA versus a control siRNA were used as the second dataset (Mitsuishi et al. 2012, Cancer Cell 22:66-79, doi:10.1016/j.ccr.2012.05.016). High-confidence Nrf2 targets (n=244) derived by (Malhotra et al. 210, Nucleic Acids Res 38:5718-5734, doi:10.1093/nar/gkq212) from integrated analyses of microarray gene expression and ChIP-seq data was used as the third dataset (Malhotra et al. 210, Nucleic Acids Res 38:5718-5734, doi:10.1093/nar/gkq212). All non-human gene names were mapped to human gene equivalents where needed. Genes that overlapped between two or more datasets were included in the high-confidence Nrf2 core target signature and used in downstream analyses. Differential gene expression analyses were conducted in R.

Gene Expression Signature Analyses

Illumina HiSeq 2000 50-nt single-ended reads were mapped to the UCSC mm9 mouse genome build (http://genome.ucsc.edu/) using RSEM (Li et al. (2011, BMC Bioinformatics 12, 323, doi:10.1186/1471-2105-12-323). Raw estimated expression counts were upper-quartile normalized to a count of 1000 (Bullard et al. (2010, BMC Bioinformatics 11, 94, doi:10.1186/1471-2105-11-94). Keap1 mutant (n=2), WT (n=2), and WT plus SFN treated (n=2) samples were jointly analyzed to derive a murine signature of Keap1 mutant gene expression changes. Given the complexity of the database in terms of a mixture of genotypes and treatment, a high-resolution signature discovery approach (Independent Component Analysis) was employed to characterize global gene expression profiles, as described previously (Papagiannakopoulos et al. (2016, Cell Metab 24, 324-331, doi:10.1016/j.cmet.2016.07.001); Li et al. (2015, Genes Dev 29, 1850-1862, doi:10.1101/gad.267393.115); Dimitrova et al. (2016, Cancer Discov 6, 188-201, doi:10.1158/2159-8290.CD-15-0854). This unsupervised blind source separation technique was used on this discrete count-based expression dataset to elucidate statistically independent and biologically relevant signatures. ICA is a signal processing and multivariate data analysis technique in the category of unsupervised matrix factorization methods. Conceptually, ICA decomposes the overall expression dataset into independent signals (gene expression patterns) that represent distinct signatures. High-ranking positively and negatively correlated genes in each signature represent gene sets that drive the corresponding expression pattern (in either direction). Each signature is thus two-sided, allowing for identification of up-regulated and down-regulated genes for each signature within each sample. Formally, utilizing input data consisting of a genes-samples matrix, ICA uses higher order moments to characterize the dataset as a linear combination of statistically independent latent variables. These latent variables represent independent components based on maximizing non-gaussianity, and can be interpreted as independent source signals that have been mixed together to form the dataset under consideration. Each component includes a weight assignment to each gene that quantifies its contribution to that component. Additionally, ICA derives a mixing matrix that describes the contribution of each sample towards the signal embodied in each component. This mixing matrix can be used to select signatures among components with distinct gene expression profiles across the set of samples. The R implementation of the core JADE algorithm (Joint Approximate Diagonalization of Eigenmatrices) (Biton et al. (2013, MineICA: Independent component analysis of transcriptomic data); Nordhausen et al. (2012, JADE: JADE and other BSS methods as well as some BSS performance criteria (R package version); Rutledge et al. (2013, Independent Components Analysis with the JADE algorithm. TrAC Trends in Analytical Chemistry 50, 22-32) was used along with custom R utilities. Statistical significance of biologically relevant signatures was assessed using the Mann-Whitney-Wilcoxon test (alpha=0.05). A murine Keap1 mutant signature was derived from this analysis, identifying genes with a differential expression pattern between wild-type and all other samples. Genes from the resulting signature with |z-score|>2 were used in subsequent Kaplan-Meier and Cox regression survival analyses. Similarly, expression profiles from the TCGA human lung-adenocarcinoma cohort were analyzed to derive a KEAP1 mutant gene expression signature. Utilizing mutation calls from TCGA (MAF files), patient primary tumor samples with protein altering mutations in KEAP1 (n=79) and wild-type KEAP1 (n=380) were identified. A combined dataset of these samples was analyzed (utilizing ICA) to detect a statistically significant expression pattern (Mann-Whitney-Wilcoxon test) separating mutant from wild-type samples. Genes from the resulting signature with |z-score|>2 were used in subsequent Kaplan-Meier and Cox regression survival analyses. All RNA-seq analyses were conducted in the R Statistical Programming language (http://www.r-project.org/). Gene set enrichment analysis (GSEA) was carried out using the pre-ranked mode with default settings (Subramanian et al. (2005, Proc Natl Acad Sci USA 102, 15545-15550, doi:10.1073/pnas.0506580102).

Results

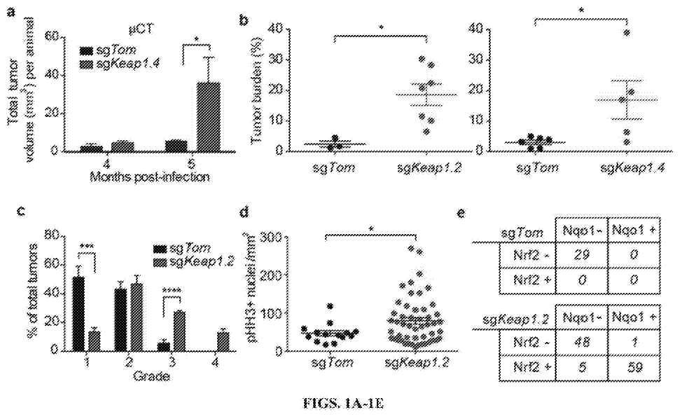

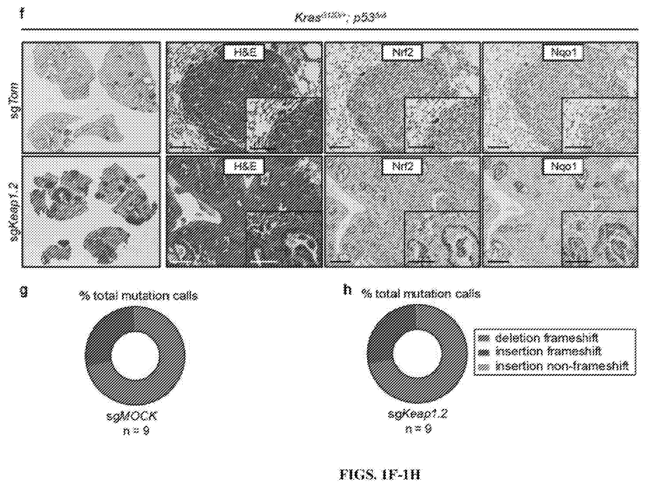

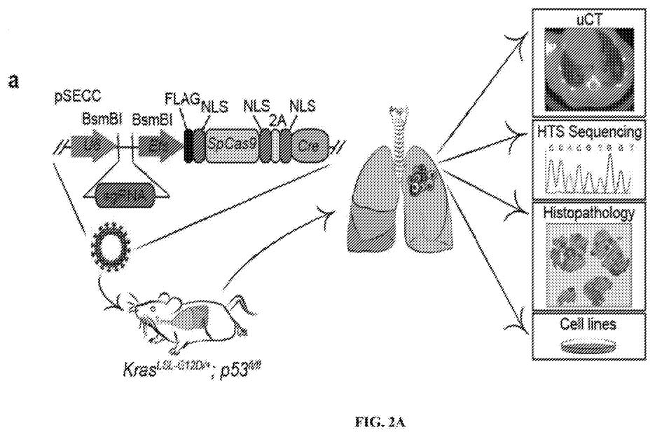

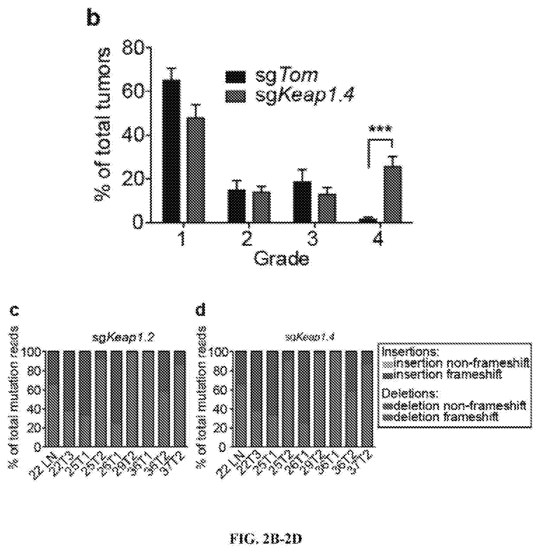

To define the role of Keap1 mutations in LUAD, KP mice were intratracheally infected with pSECC lentivirus expressing sgRNAs against Keap1 or Tdtomato as a control (FIG. 2A). sgKeap1 mice (n=5) contained significantly larger overall tumor volume and faster growth kinetics compared to sgTom mice (n=3) based on longitudinal micro-computed tomography (micro-CT; p<0.05; FIG. 1A). In line with micro-CT data, histological assessment of tumor burden revealed a significant increase in tumor burden in sgKeap1 tumors compared to controls (p<0.05 FIG. 1B). In addition, histopathological assessment revealed a dramatic increase in high-grade 3 and 4 tumors (FIG. 1C and FIG. 2B, p<0.0001 for sgKeap1.2 grade 3 and p<0.001 for sgKeap1.4 grade 4) in sgKeap1 tumors compared to controls. Furthermore, sgKeap1 tumors displayed an increase in pHH3-positive nuclei compared to controls (FIG. 1D p<0.05) indicating an increase in mitotic index.

To gain insight on how LOF of Keap1 accelerates lung tumorigenesis, immunohistochemical (IHC) analyses were performed to assess whether loss of Keap1 led to increased nuclear localization of Nrf2 protein and cytoplasmic levels of its target gene Nad(p)h dehydrogenase quinone 1 (Nqo1). It was observed that the majority (60%) of sgKeap1 tumors had increased nuclear localization of Nrf2 and dramatically higher levels of Nqo1 as compared to controls (p<0.0001, FIG. 1E, 1F). Importantly, based on analysis of serially sectioned lung tumors, nearly all tumors that stained positive for nuclear Nrf2 also contained higher levels of Nqo1, (p<0.0001, FIG. 1E). These data demonstrate that cell autonomous Keap1 LOF mutations lead to increased stability, nuclear localization and transcriptional activity of Nrf2 in KP tumors.

To determine the nature of mutations at the Keap1 locus, high-throughput DNA sequencing of micro-dissected sgKeap1 tumors (sgKeap1.2 and sgKeap1.4) was performed. As expected (Sanchez-Rivera et al., “Rapid Modelling of Cooperating Genetic Events in Cancer Through Somatic Genome Editing,” Nature 516:428-431 (2014), which is hereby incorporated by reference in its entirety), tumors that stained positive for nuclear Nrf2 and Nqo1 contained frameshift LOF insertions or deletions (indels) in Keap1 upstream of the PAM site (FIG. 1G, 1H and FIG. 2C, 2D). Additionally, a clonal enrichment of Keap1 LOF alleles from a subclonal fraction in the primary tumor to a clonal fraction in related lymph node metastasis in the same animal was observed, supporting this CRISPR/Cas9-editing system for assessing clonal relationships between primary tumors and metastases (McKenna et al., “Whole-Organism Lineage Tracing by Combinatorial and Cumulative Genome Editing,” Science 353:aaf7907 (2016), which is hereby incorporated by reference in its entirety) (FIG. 2E-2G).

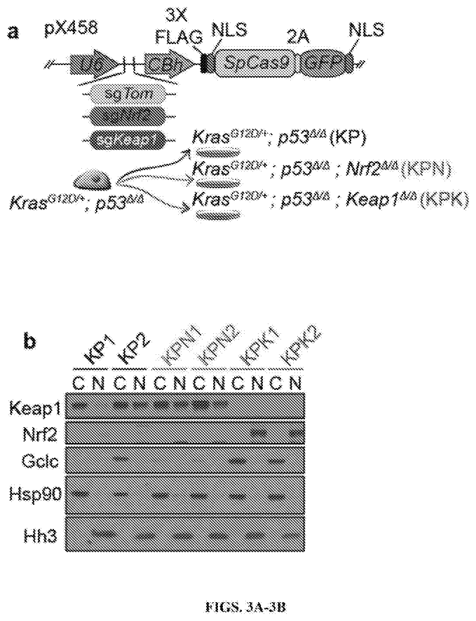

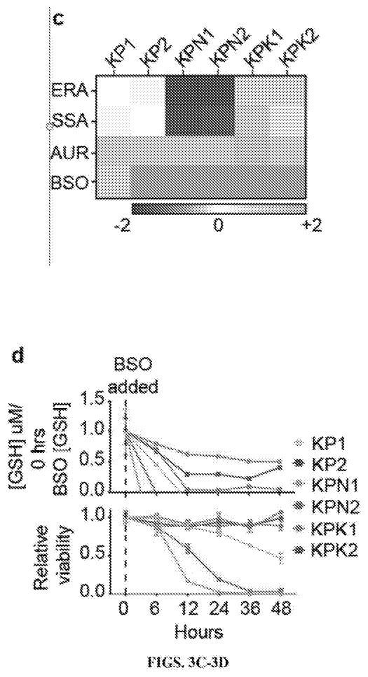

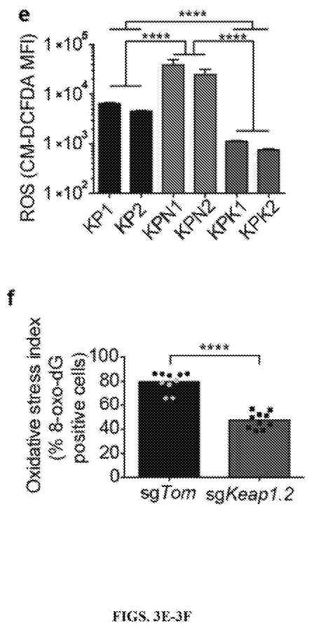

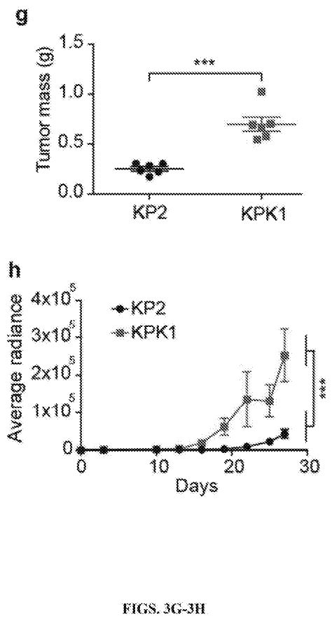

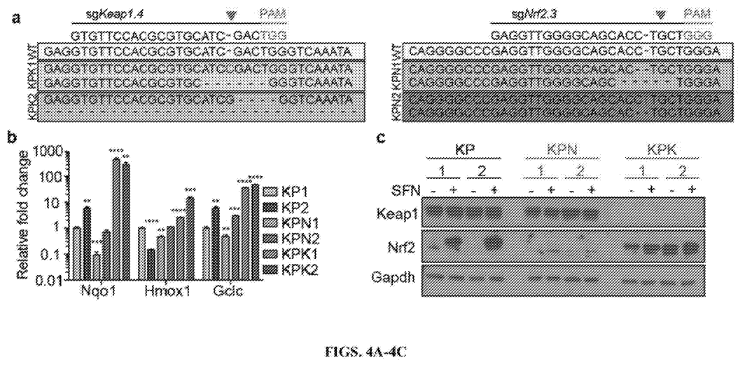

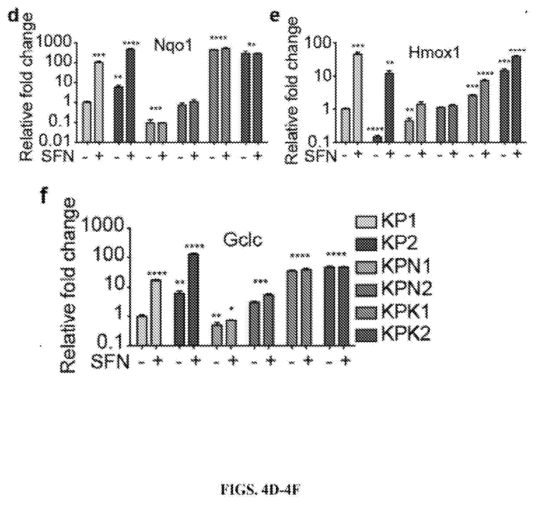

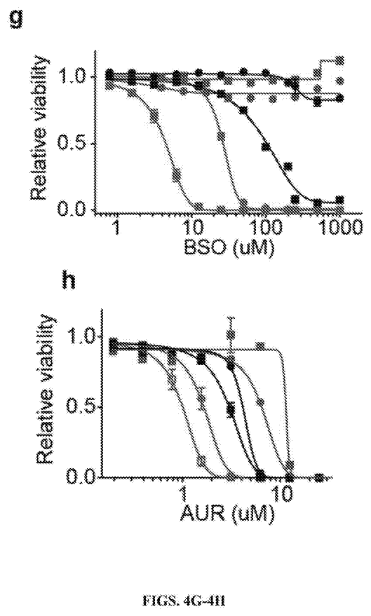

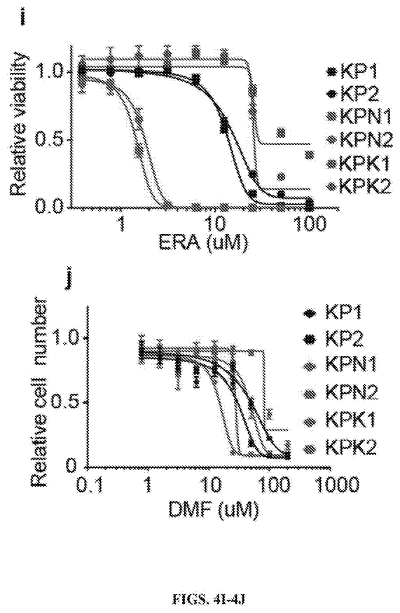

To determine the cell-autonomous role of Nrf2 and Keap1 in regulating cellular growth and regulation of antioxidant pathways, isogenic KP GEMM-derived lung tumor cell lines were generated with CRISPR/Cas9-induced LOF mutations in Nrf2 (KPN), Keap1 (KPK), and sgTom controls (KP) (FIG. 3A and FIG. 4A; n=2 cell lines per genotype). As expected, loss of Keap1 in KPK cells, led to increased nuclear Nrf2 and increased levels of Nrf2 targets both by protein (Gclc; FIG. 3B) and gene expression (Nqo1, Hmox1 and Gclc; FIG. 4B). In addition, Sulforaphane (SFN), a well-established inducer of Nrf2, stabilized Nrf2 protein levels and expression of target genes in KP cells but not in KPK or KPN cells (FIG. 4C-4F).

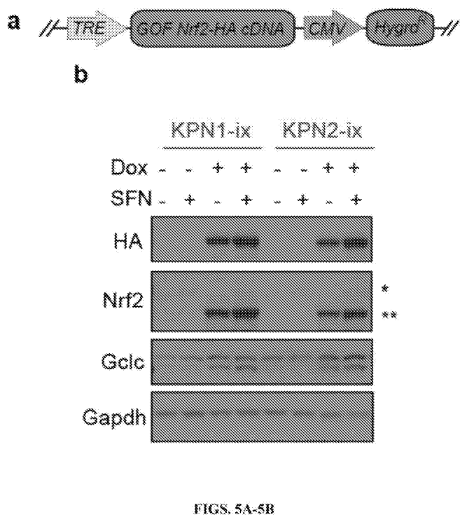

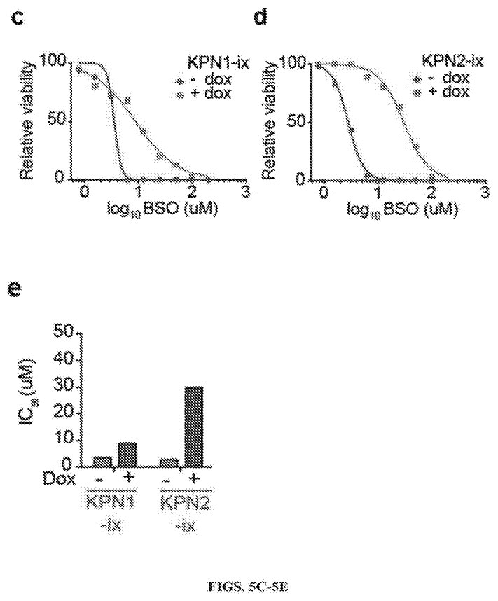

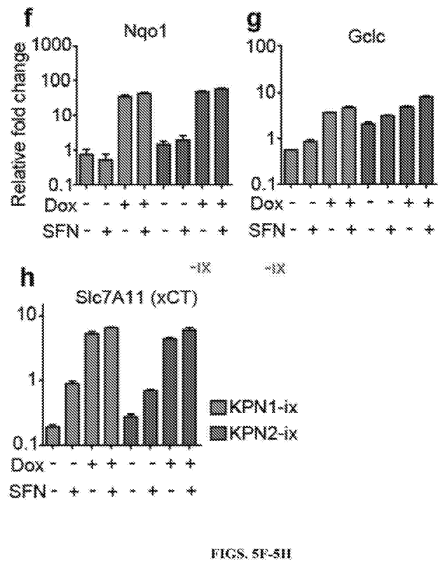

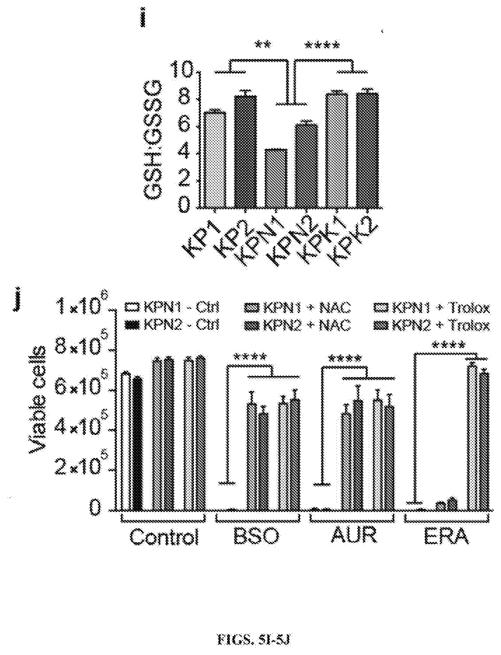

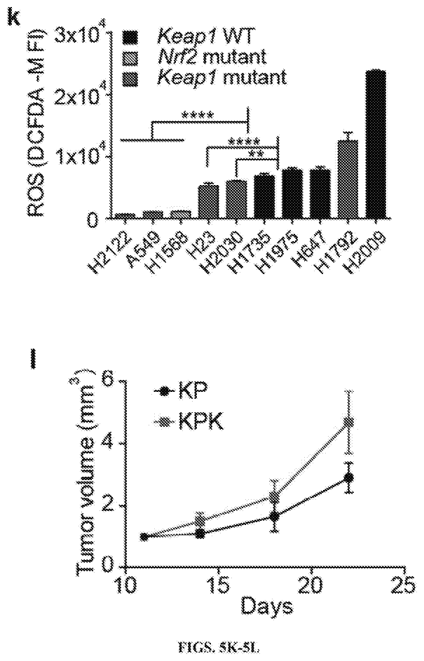

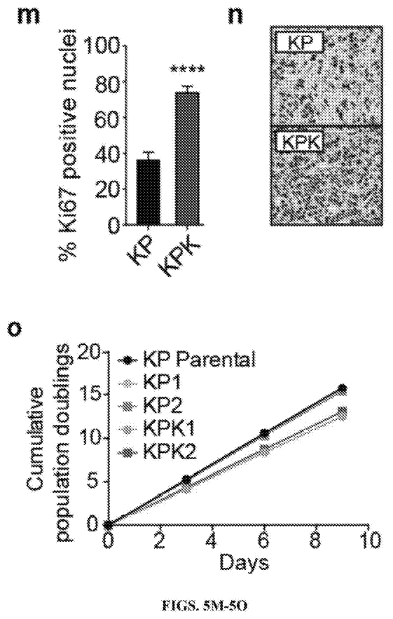

It was next demonstrated that treatment with the glutamate-cysteine ligase (GCL) inhibitor, L-buthionine-sulfoximine (BSO), the cystine/glutamate antiporter (SLC7A11/xCT) inhibitor, erastin (ERA), or the thioredoxin reductase (TXNRD1) inhibitor, auranofin (AUR) yielded distinct genotype-specific sensitivities in the cell lines (FIG. 3C and FIG. 4G-4I). Nrf2 loss resulted in a dramatic decrease in cell viability, while Keap1 loss enhanced cellular survival in the presence of all compounds (FIG. 3C and FIG. 4G-4J). To demonstrate that resistance to oxidative stress inducers is Nrf2-dependent, KPN cell lines expressing a doxycycline inducible promoter driving expression of a GOF-Nrf2 cDNA that lacks exon 2 of Nrf2 that encodes the Neh2 Keap1 association domain (Goldstein et al., “Recurrent Loss of NFE2L2 Exon 2 Is a Mechanism for Nrf2 Pathway Activation in Human Cancers,” Cell Rep 16:2605-2617 (2016), which is hereby incorporated by reference in its entirety) (KPN-ix, FIGS. 5A and 5B) were generated. Induction of GOF-Nrf2 rescued BSO-mediated loss of viability by promoting activation of multiple Nrf2 target genes (FIG. 5C-5H). The addition of BSO to the panel of isogenic cell lines revealed a stark contrast in the reduced glutathione (GSH): oxidized glutathione (GSSG) ratio (FIG. 51). KPN clones displayed rapid exhaustion of GSH pools after 12 hours of BSO treatment which correlated with the decrease in viability. In contrast, KPK counterparts were able to maintain measurable levels of GSH and viability compared to KP clones (FIG. 3D). The loss of viability in KPN cells in response to oxidative stress agents was rescued by treatment with the antioxidants N-acetyl cysteine or Trolox linking the inability to quench ROS in KPN cells to cell death (FIG. 5J). In line with this, KPK clones, displayed marked lower ROS level (FIG. 3E). Furthermore, it was observed that KEAP1 mutant human LUAD cells exhibited decreased ROS levels compared to KEAP1 wild-type (WT) cells (FIG. 5K). Consistently, sgKeap1 tumors displayed a decrease in the oxidized DNA lesion, 8-Oxo-2′-deoxyguanosine (8-oxo-dG), compared to sgTom tumors (FIG. 3F). Interestingly, KPK cells grew faster than KP cells in vivo but not in vitro (FIG. 3G, 3H and FIG. 5L-5O), suggesting a differential requirement of the antioxidant Nrf2 response during tumorigenesis in vivo. These data indicate that Nrf2 levels dictate the differential antioxidant response to oxidative stress and that high Nrf2 levels gives lung cancer cells a selective growth advantage in vivo but not necessarily in vitro.

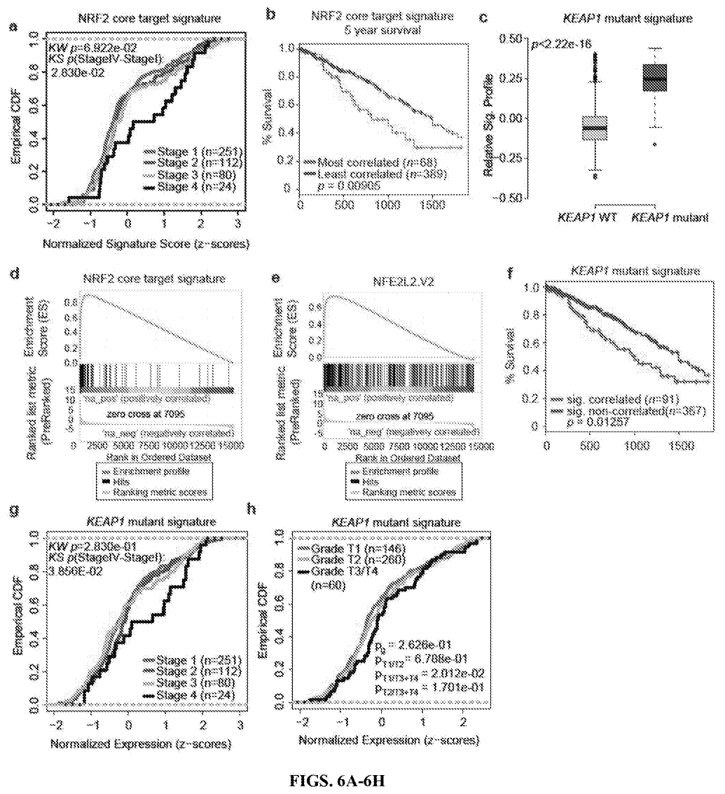

These data support the tumor suppressive role of Keap1 in a Kras-driven GEMM of lung cancer. To explore the implications of these data for human lung cancer, The Cancer Genome Atlas (TCGA) data for KRAS-mutant LUAD (Cancer Genome Atlas Research, “Comprehensive Molecular Profiling of Lung Adenocarcinoma,” Nature 511:543-550 (2014), which is hereby incorporated by reference in its entirety) was analyzed. Patients with KRAS and KEAP1 mutant tumors displayed significantly less median survival (˜3 years; p<0.01; FIG. 6B) and their tumors had an enrichment in multiple antioxidant pathways associated and the NRF2 oncogenic signature (FIG. 6C-E) compared to KRAS mutant patients with WT KEAP1 tumors. A core signature of 108 high confidence NRF2 target genes was then derived, based on published datasets (Mitsuishi et al., “Nrf2 Redirects Glucose and Glutamine into Anabolic Pathways in Metabolic Reprogramming,” Cancer Cell 22:66-79 (2012); Singh et al., “RNAi-Mediated Silencing of Nuclear Factor Erythroid-2-Related Factor 2 Gene Expression in Non-Small Cell Lung Cancer Inhibits Tumor Growth and Increases Efficacy of Chemotherapy,” Cancer Res. 68:7975-7984 (2008); Malhotra et al., “Global Mapping of Binding Sites for Nrf2 Identifies Novel Targets in Cell Survival Response Through ChIP-Seq Profiling and Network Analysis,” Nucleic Acids Res. 38:5718-5734 (2010), each of which is hereby incorporated by reference in its entirety) and an enrichment of these genes in LUAD tumors from advanced stage IV disease (p<0.05; FIG. 6) and in KRAS; KEAP1 mutant LUAD (FIG. 6D) was observed, which further validates the potent increase of the NRF2 transcriptional signature in KEAP1 mutant LUAD. Finally, this signature is independently prognostic in stratifying patients based on survival (HR=1.78; p<0.05; FIG. 6F). Furthermore, high grade tumors (grades III/IV) and late stage tumors (clinical stage IV disease) were significantly enriched for the human KEAP1 mutant transcriptional signature (grade III/IV: p=0.02; stage IV: p=0.038; APPLICATION FIG. 6G-H). Importantly, this signature was found to be independently prognostic in the TCGA LUAD cohort while controlling for other clinical covariates in a Cox proportional hazards model (HR=1.22; univariatep=0.029, multivariablep=0.04) where higher enrichment for the signature was associated with significantly worse survival. These data are consistent with KEAP1 mutations cooperating with mutant-KRAS to promote cancer progression in LUAD patients.

As indicated herein, the translational potential of our GEMM results to human LUAD with KEAP1 mutations was assessed by performing RNA-seq of KP and KPK cell lines. It was observed that the Keap1 mutant GEMM signature was prognostic of patient survival in human LUAD (FIG. 6) and was enriched in KRAS; KEAP1 mutant LUAD, including multiple NRF2-driven antioxidant pathways (FIG. 6). These results demonstrate that the findings in GEMM recapitulate human LUAD patients with KEAP1 mutations, validating the use of this GEMM for uncovering druggable genotype-specific vulnerabilities in KRAS; KEAP1 mutant LUAD.

In order to identify therapeutic vulnerabilities in KRAS; KEAP1 mutant LUAD, a focused CRISPR/Cas9-based genetic screen was performed to identify synthetic genetic interactions with Keap1 mutations. A pool of lentiviruses was generated expressing a CRISPR/Cas9 library of sgRNAs against Nrf2 targets and genes implicated in the Nrf2 antioxidant response (17 genes and 3 controls, 3-4 sgRNAs/gene, FIG. 7A and FIG. 8A) and infected KP or KPK cells (n=2 per genotype). The relative depletion of sgRNAs was assessed to identify genes in which mutations selectively affected the growth of KPK compared to KP cells. Notably, sgRNAs (n=3) against solute carrier family 1 member 5 (Slc1a5), a glutamine transporter, were depleted in KPK but not KP cells, suggesting that Slc1a5 is a synthetic lethal hit in Keap1 mutant cells (APPLICATION FIG. 7A and FIG. 8A). Consistently, sgSlc1a5 transduced Kras; Keap1 mutant mouse (KPK1, KPK2) and human (A549 and H2030) cell lines displayed markedly stunted growth with no effect in Keap1 wildtype mouse (KP1, KP2) and human (H2009 and H647) cell lines (FIG. 7B-7D and FIG. 8B). Furthermore, KPK cell lines treated with GPNA, a small molecule inhibitor of Slc1a5, displayed an increased sensitivity compared to KP cell lines (FIG. 7E and FIG. 8C). The selective requirement of Slc1a5 for growth of KPK cell lines suggested a metabolic dependency of KPK cells to glutamine. Decreasing glutamine concentration in the media led to a robust suppression of growth in KPK cell lines with little effect on KP cell lines (FIG. 7F and FIG. 8D). The metabolic dependency of KPK cell lines on Slc1a5 and glutamine suggest that glutamine is used for anaplerosis in the context of an increased glycolytic state (Metallo et al., “Reductive Glutamine Metabolism by IDH1 Mediates Lipogenesis Under Hypoxia,” Nature 481:380-384 (2011), which is hereby incorporated by reference in its entirety). Indeed, both KPK cell lines had higher glucose (FIG. 8E) and glutamine consumption (FIG. 7G) coupled with a marked increase in lactate excretion compared to KP cells (FIG. 8E) and increased sensitivity to the glycolytic inhibitor 2-deoxy-D-glucose (2-DG; FIG. 8F, 8G). These results suggest that KPK cells are more glycolytic and depend more on glutamine than KP cells.

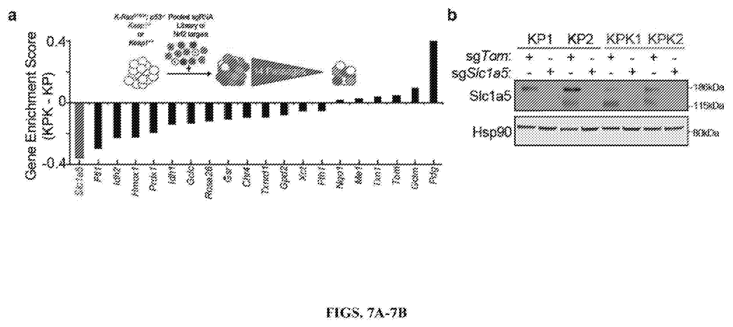

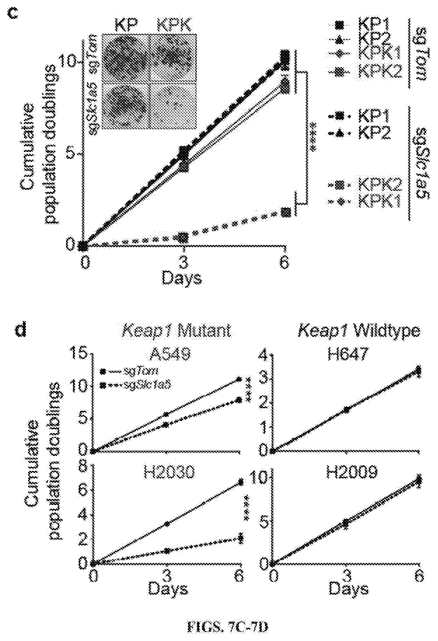

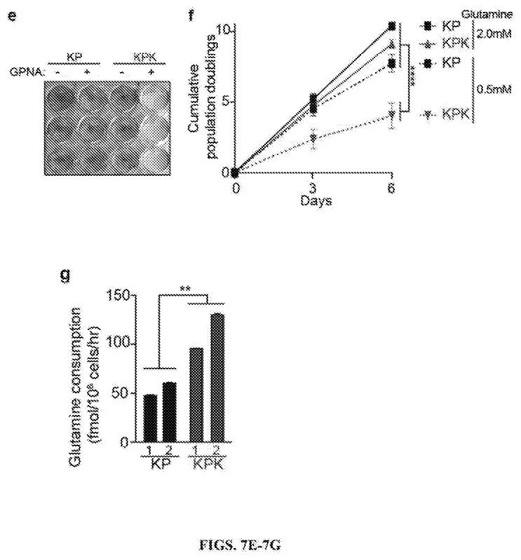

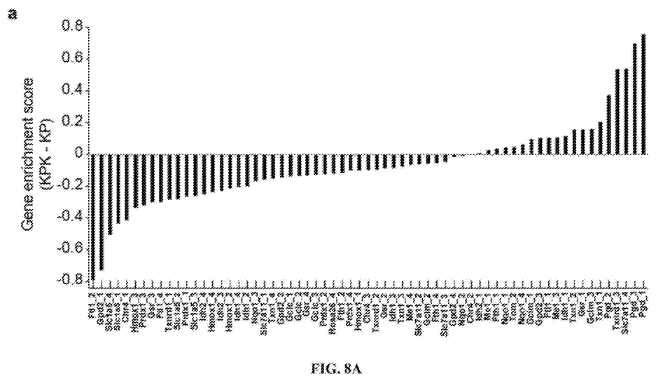

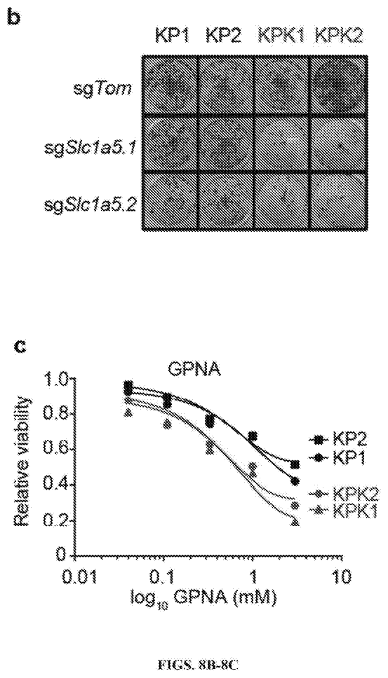

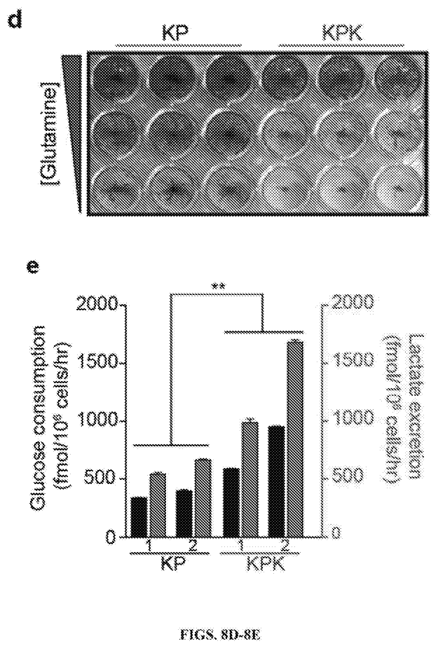

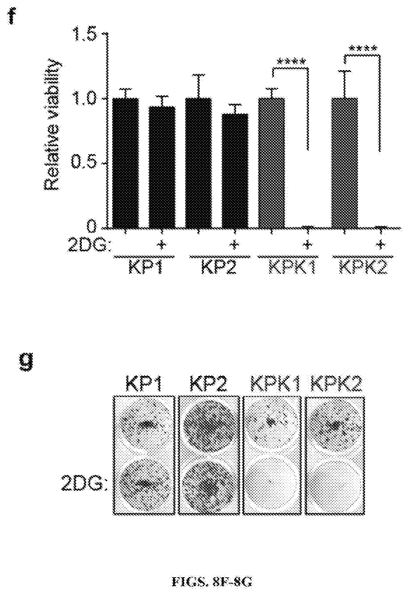

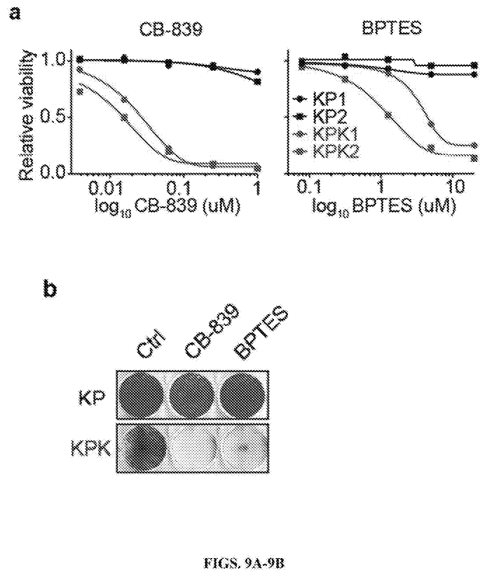

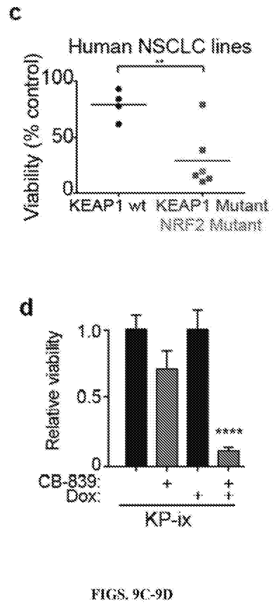

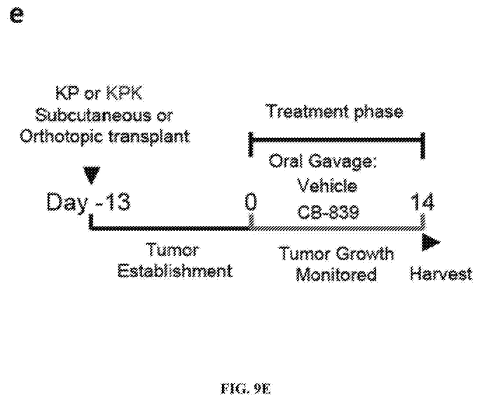

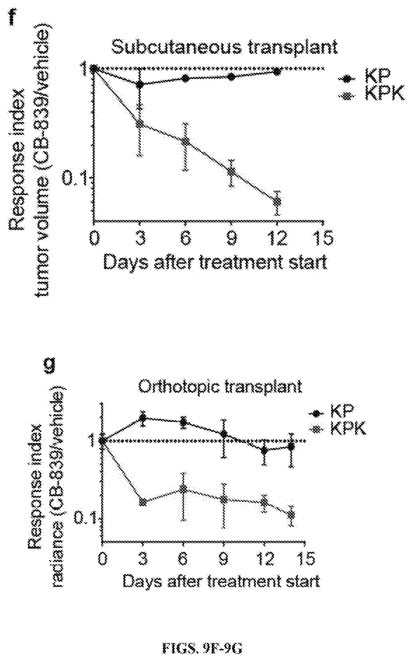

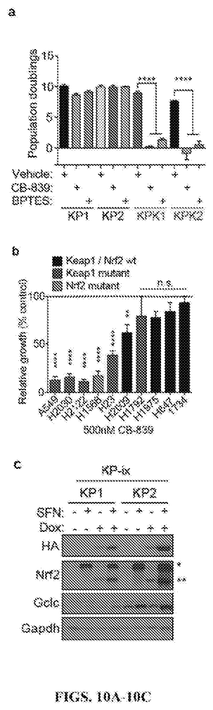

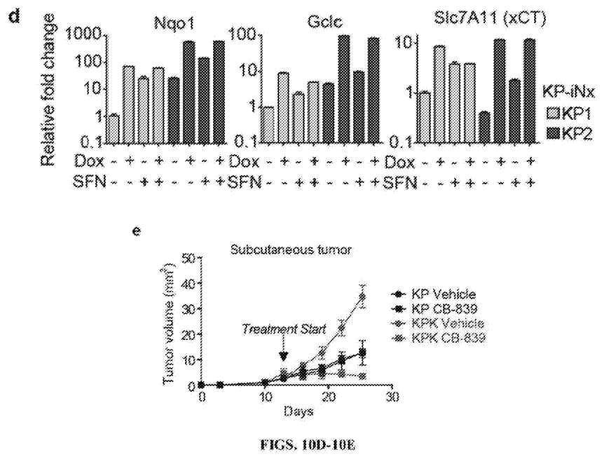

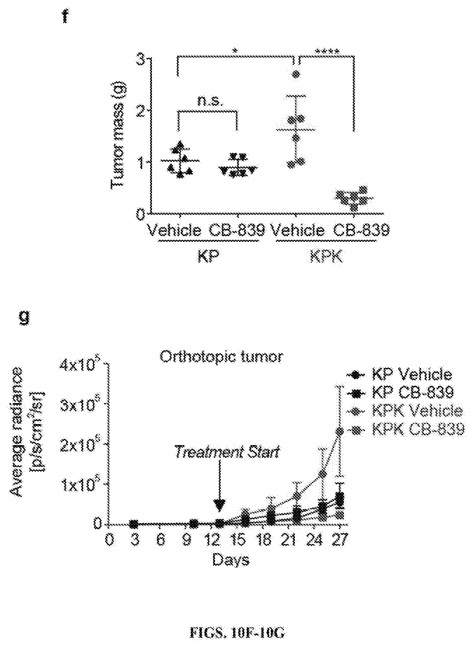

It was next asked if increased glutamine utilization in KPK cell lines could be a druggable metabolic liability. KPK cell lines displayed a striking sensitivity to two independent small molecule inhibitors of glutaminase, the rate limiting enzyme for glutamine utilization in the cell (FIG. 9A, 9B and FIG. 10A). Consistently, a panel of human lung cancer cells containing KEAP1 mutations or GOF mutations in NRF2 were sensitive to glutaminase inhibition while KEAP1 WT cells were largely resistant (FIG. 9C and FIG. 10B). To demonstrate that the sensitivity of KPK cells to glutaminase inhibitor is dependent on hyperactive Nrf2 signaling, the -ix vector (FIG. 5A) was introduced to KP cells (KP-ix, FIG. 10C, 10D). Induction of GOF-Nrf2 induced robust sensitivity to CB-839 (FIG. 9D). To investigate the therapeutic potential of targeting glutaminase in Keap1 mutant tumors in vivo, KP and KPK cells were transplanted subcutaneously and orthotopically (lung) in immunodeficient animals. Once tumors had established, treatment was initiated with either vehicle or CB-839, an orally bioavailable glutaminase inhibitor in phase I clinical trials for KRAS mutant NSCLC (FIG. 9E). In line with an earlier study (Davidson et al., “Environment Impacts the Metabolic Dependencies of Ras-Driven Non-Small Cell Lung Cancer,” Cell Metab 23:517-528 (2016), which is hereby incorporated by reference in its entirety), KP-derived tumors exhibited no response to CB-839 treatment (FIG. 9F, 9G and FIG. 10E-10G). In contrast, KPK-derived subcutaneous and orthotopic tumors showed a dramatic stunted growth over the course of the experiment, and established smaller final tumor weights in response to CB-839 (FIG. 9F, 9G and FIG. 10E-10G). Taken together, these data suggest that glutaminase is a viable target for inhibiting Keap1/Nrf2 mutant lung adenocarcinoma and might predict treatment response to glutaminase inhibitors in patients.

Collectively, this study provides the first example of a CRISPR/Cas9-based precision medicine approach that both defines the functional role of putative cooperating genetic events in lung cancer and subsequently identifies genotype-specific vulnerabilities. Using this approach it is shown that Keap1 mutations cooperate with mutant Kras to drive LUAD progression and provide a rationale for sub-stratification of LUAD patients harboring mutations in KRAS; KEAP1 or NRF2 as treatment responders to glutaminase inhibitors.

### Example 2—Additional Keap1 Data

### Loss of Keap1 Accelerates Lung Tumorigenesis

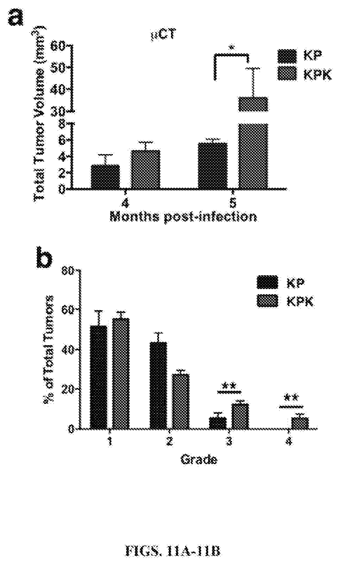

To assess whether Keap1 is a tumor suppressor in lung adenocarcinoma, the pSECC somatic genome editing system (Sanchez-Rivera et al., “Rapid Modeling of Cooperating Genetic Events in Cancer Through Somatic Genome Editing,” Nature (2014), which is hereby incorporated by reference in its entirety) was utilized to perform LOF of Keap1 in KP tumors in vivo. KP animals were infected intratracheally with a pSECC lentivirus expressing an sgRNA against Keap1 (KPK) previously validated in vitro or control sgRNA (KP). It was observed that KPK animals contain a significantly larger overall tumor volume at 5 months post-initiation (FIG. 11A). Furthermore, when comparing tumor burden from 4 to 5 months there is an average 6-8 fold increase in tumor size in KPK tumors, compared KP animals. Histological assessment of tumor grade revealed a significant increase in aggressive grade 3 and 4 tumors in KPK mice (FIG. 11B). These data suggest that LOF of Keap1 by CRISPR somatic editing in KP tumors leads to increased tumor growth, pointing to a tumor suppressive role of Keap1.

KEAP1/NRF2 Status Correlates with Poor Clinical Outcome

The GEMM data indicates that LOF of Keap1 in KP tumors leads to increased Nrf2 activity and increased tumor growth, pointing to a tumor suppressive role of Keap1. To further validate the findings from the GEMM, publically available data for KRAS mutant lung adenocarcinomas from The Cancer Genome Atlas (TCGA) was analyzed. It was observed that patients with KRAS and KEAP1 mutant tumors have significantly less median survival than patients with KRAS mutant and KEAP1 wild-type tumors (˜3 years; p<0.01; FIG. 11). This result further confirms the findings in the GEMM, suggesting that KEAP1 mutations may cooperate with KRAS to promote lung cancer progression.

Based on several published datasets on NRF2 transcriptional targets, a core signature of 108 high confidence target genes was derived. Using TCGA tumor RNAseq data from patients at different clinical stages (stage I, II, III, IV) of lung adenocarcinoma, it was observed that expression of these 108 genes is significantly enriched in tumors from stage IV patients with advanced disease (p<0.05; FIG. 12B). These data suggest that NRF2 target gene expression correlates with advanced disease.

### Generation of Isogenic Keap1 and Nrf2 Null Murine Lung Cancer Cell Lines

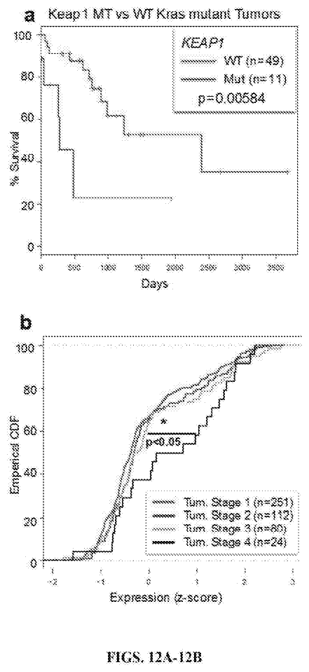

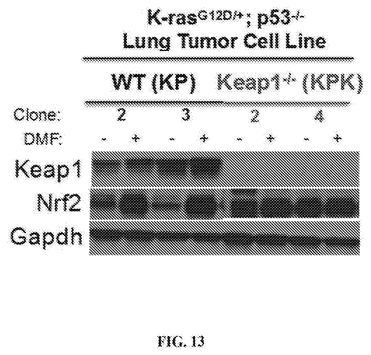

To perform in vitro functional characterization of and Keap1, isogenic cell lines were generated from a Kras^{G12D/+}; p53^{-/-} C57B6 GEMM-derived cell line using the CRISPR/Cas9 and sgRNAs against Keap1. LOF of the gene was validated by western blot analysis in basal conditions and in the presence of an oxidative stress inducer, dimethyl fumarate (DMF), which is known to deplete the cellular glutathione pool and induce Nrf2 activation (Sullivan et al., “The Proto-oncometabolite Fumarate Binds Glutathione to Amplify ROS-Dependent Signaling,” Molecular Cell 51:236-248 (2013), which is hereby incorporated by reference in its entirety). It was observed that Nrf2 is induced in control cells in response to 6 hour DMF treatment. Keap1 LOF leads to a robust increase of Nrf2 even in the absence of DMF (FIG. 13). These results demonstrate efficient CRISPR/Cas9-based knockout LOF of Nrf2 or Keap1 in multiple isogenic lines that were generated.

### Generation of Isogenic Keap1 and Nrf2 Null Murine Lung Cancer Cell Lines

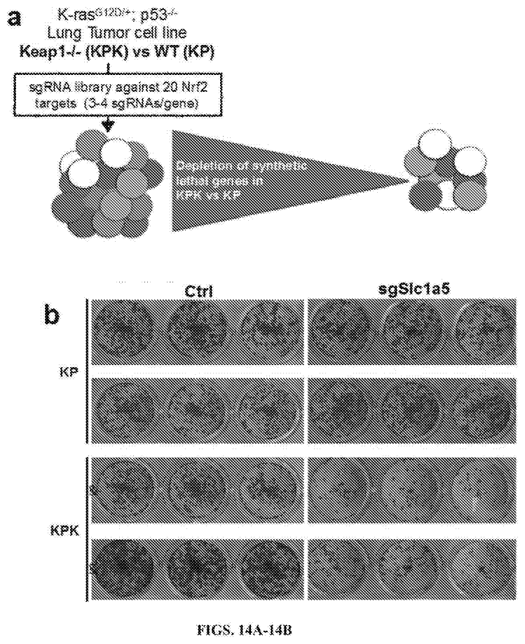

In order to identify synthetic lethal interactions in Keap1 mutant KP cells, a focused CRISPR/Cas9-based genetic screen was performed targeting 20 bona-fide Nrf2 targets genes, with using 4 different sgRNAs against each gene (FIG. 14A). The screen was performed using a pooled lentivirus format in order to deliver the CRISPR/Cas9 system into KP or KPK cells. To identify genes selectively required for the loss of KPK and not KP cells, the relative depletion of sgRNAs was assessed after culturing cells for 15 passages. Three out of 4 sgRNAs against the amino acid transporter, Slc1a5, were depleted in KPK but not KP cells, suggesting a synthetic lethal interaction of Slc1a5 with Keap1 mutation. This synthetic lethal interaction result was further validated in multiple KP and KPK cells using the sgRNA that scored as most depleted in the screen (FIG. 14B).

### Keap1 Mutant Cells Require Uptake of Serine and Glutamine

Slc1a5 is an amino acid transporter that mediated the uptake of serine and glutamine from the extracellular environment. The selective requirement of Slc1a5 for growth of KPK cells suggested a metabolic dependency of KPK cells to serine and glutamine. To test this hypothesis, KP and KPK cells were grown in media lacking serine or media with decreasing amounts of glutamine. It was observed that depletion of serine led to a dramatic attenuation in the growth of KPK with no effect on KP cells (FIG. 15A). Decreasing glutamine concentration (0.5 mM) in the media led to a robust suppression of growth in KPK with little to no effect on KP cells (FIG. 15B). These results provide additional support for the important function of Slc1a5 in KPK cells as a source of serine and glutamine, which presents a metabolic liability of KPK cells.

Generation of Dual sgRNA Inducible CRISPR/Cas9 System

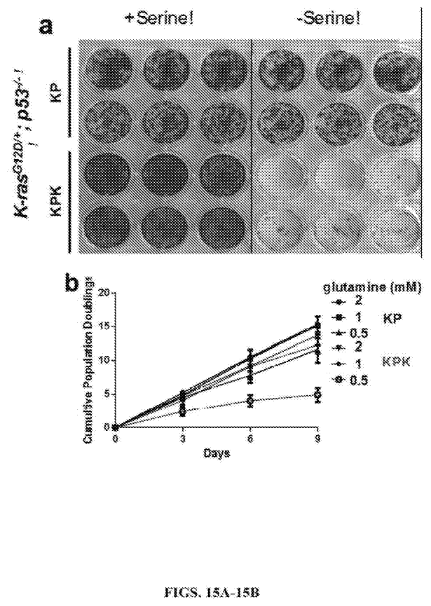

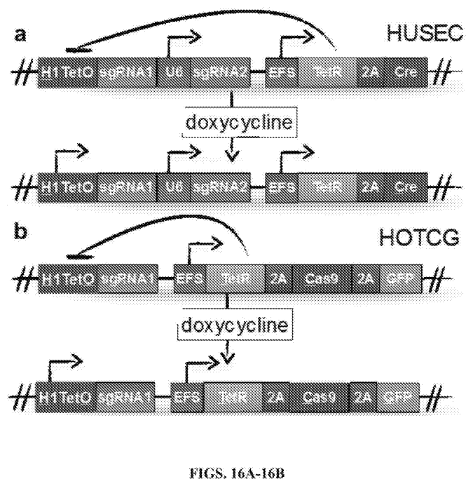

Using extensive expertise in genome engineering tool development, lentiviral vectors were generated that simultaneously express two sgRNAs, one inducible from an H1tetO promoter and another constitutive from a U6 promoter. In addition the vector constitutively expresses Cre recombinase and a tetR that silences the H1tetO promoter in the absence of doxycycline (FIG. 16A) (Aubrey et al., “An Inducible Lentiviral Guide RNA Platform Enables the Identification of Tumor-Essential Genes and Tumor-promoting Mutations In Vivo,” Cell Reports 10:1422-1432 (2015), which is hereby incorporated by reference in its entirety). Using this approach, HUSEC vectors constitutively expressing a Keap1 or control sgRNA can be generated and simultaneously be able to temporally induce expression of an sgRNA against Slca1a5 in the KP GEMM. This system enables the mediation LOF of Keap1 at tumor initiation and once tumors have established, and the introduction of doxycycline to induce expression of another sgRNA against Slc1a5 to mediate its loss.

To be able to stably introduce an all-in-one inducible CRISPR/Cas9 system, HOTCG, a vector system that stably expresses Cas9, GFP and TetR silences the H1tetO promoter in the absence of doxycycline (FIG. 16B), was generated. Upon infection with this lentivirus cells can be isolated by fluorescence-associated cell sorting (FACS) and in the presence of doxycycline the sgRNA is expressed to drive CRISPR/Cas9-editing.

### Example 3—GPD2 Study

### Genetic Screen in Isogenic Keap1 Mutant and WT Kras-Driven Murine Lung Cancer Cell Lines

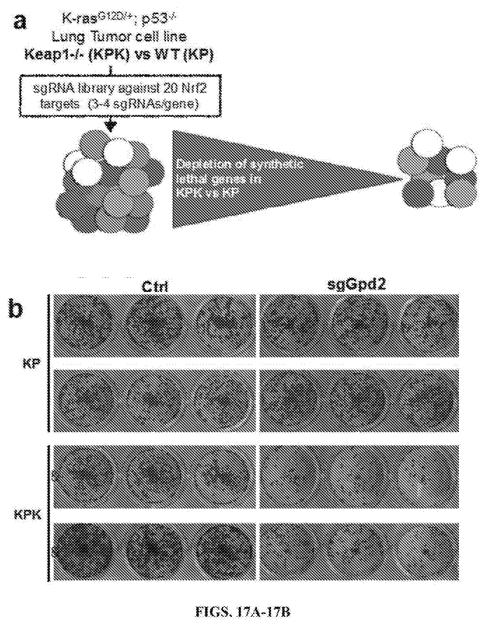

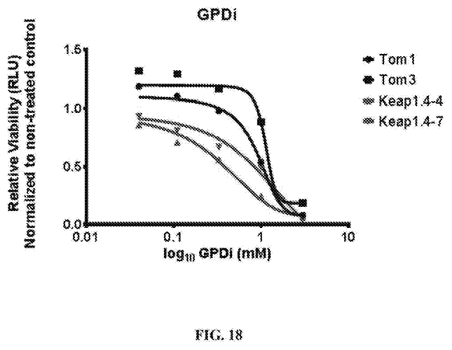

In order to identify synthetic lethal interactions in Keap1 mutant KP cells, a focused CRISPR/Cas9-based genetic screen targeting 20 bona-fide Nrf2 targets genes was performed using 4 different sgRNAs against each gene (FIG. 17A). The screen was performed using a pooled lentivirus format in order to deliver the CRISPR/Cas9 system into KP or KPK cells. To identify genes selectively required for the loss of KPK and not KP cells, the relative depletion of sgRNAs was assessed after culturing cells for 15 passages. Three out of 4 sgRNAs against the Gpd2 were depleted in KPK but not KP cells, suggesting a synthetic lethal interaction of Gpd2 with Keap1 mutation. This synthetic lethal interaction result was further validated in multiple KP and KPK cells using the sgRNA that scored as most depleted in the screen (FIG. 17B). Furthermore, using a chemical inhibitor of Gpd2, iGP-5, sensitivity was analyzed in KPK cells as compared to KP cells (FIG. 18).

Generation of Dual sgRNA Inducible CRISPR/Cas9 System

Using extensive expertise in genome engineering tool development, lentiviral vectors have been generated that simultaneously express two sgRNAs, one inducible from an H1tetO promoter and another constitutive from a U6 promoter. In addition the vector constitutively expresses Cre recombinase and a tetR that silences the H1tetO promoter in the absence of doxycycline (FIG. 19A) (Aubrey et al. 2015, Cell reports 10:1422-1432). Using this approach, HUSEC vectors can be generated constitutively expressing a Keap1 or control sgRNA and simultaneously be able to temporally induce expression of an sgRNA against Gpd2 in the KP GEMM. This system enables the mediation of LOF of Keap1 at tumor initiation and once tumors are established, introduce doxycycline to induce expression of another sgRNA against Gpd2 to mediate its loss.

To be able to stably introduce an all-in-one inducible CRISPR/Cas9 system, HOTCG, a vector system that stably expresses Cas9, GFP and TetR silences the H1tetO promoter in the absence of doxycycline (FIG. 19B) is generated. Upon infection with this lentivirus, cells can be isolated by fluorescence-associated cell sorting (FACS) and, in the presence of doxycycline, the sgRNA is expressed to drive CRISPR/Cas9-editing.

### Example 4—Slc1a5 Studies

### Determination of the Role of Slc1a5 in Keap1 Mutant Lung Tumorigenesis Using GEMMs

As described above, Slc1a5 is required for the growth of Keap1 mutant lung cancer cells in vitro, which indicates that Slc1a5 plays a crucial role in the maintenance of KPK tumors in vivo. To further establish whether Slc1a5 is critical for Keap1 mutant lung adenocarcinoma growth and maintenance, three separate but complementary in vivo based approaches are performed: 1) Orthotopic transplantation of syngenic KP or KPK cells containing an inducible CRISPR/Cas9 editing system for Slc1a5 LOF; 2) LOF of Slc1a5 in autochthonous tumors at tumor initiation using pSECC in KP and KPK animals; 3) LOF of Slc1a5 in established autochthonous tumors.

Following the extensive in vitro characterization of isogenic KP and KPK clones with differential Slc1a5 status, their cellular growth is assessed in vivo by orthotopic intratracheal transplantation in the lungs of immunocompetent C57B6 mice. The preliminary results demonstrate that loss of Slc1a5 is required for the growth of KPK cells. Therefore it is expected that KPK cells with loss of Slc1a5 will not grow upon transplantation in vivo. Therefore, whether Slc1a5 is required for tumor maintenance is assessed by transplanting cells orthotopically in the lung, allowing tumors to form and then temporally performing LOF of Slc1a5 using previously validated sgRNAs in a doxycycline-inducible CRISPR/Cas9 editing system (Aubrey et al., “An Inducible Lentiviral Guide RNA Platform Enables the Identification of Tumor-Essential Genes and Tumor-Promoting Mutations In Vivo,” Cell Reports 10:1422-1432 (2015), which is hereby incorporated by reference in its entirety). All cells will stably express luciferase, which allows for in vivo realtime monitoring of tumor growth by non-invasive bioluminescence imaging. When animals are moribund lung tissue is collected for histopathological analysis of tumor burden and grade. To determine the effects of Keap1/Nrf2 status on different hallmarks of tumor progression, rates of proliferation and apoptosis are assessed by IHC staining of Ki67 and cleaved caspase 3, respectively. Demonstrating that Slc1a5 is essential for the ability of KPK lung cancer cells to survive and grow in vivo, statistically significant changes in tumor burden, grade and/or the levels of markers of proliferation and cell viability are expected, where KPK cells are unable to grow in the absence of Slc1a5 as compared to no significant effect or less of an effect on growth of KP cells.

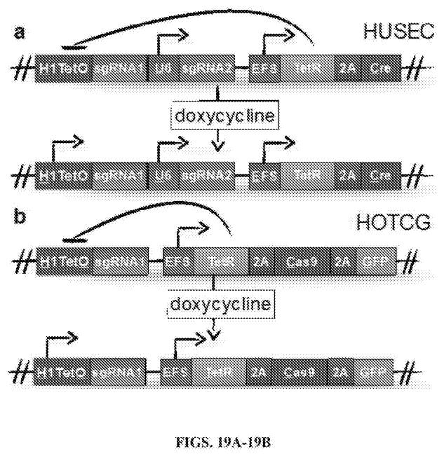

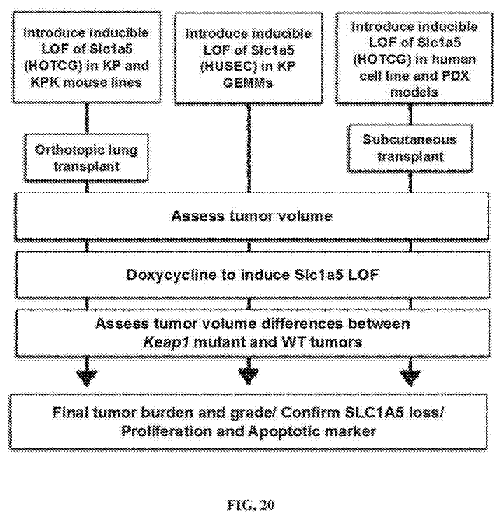

To test if Slc1a5 is required for lung tumorigenesis, LOF of Slc1a5 is performed using CRISPR/Cas9-based somatic editing in KP tumors utilizing a similar but alternative approach to the pSECC system. KPC9 (Kras^{LSL-G12D/+}; p53^{flox/flox}; Rosa26^{LSL-Cas9-2A-GFP/LSL-CAS9-2A-GFP}) animals are utilized which express a Cre-inducible version of Cas9 as well as GFP from the ubiquitously expressed Rosa26 locus (Platt et al., “CRISPR-Cas9 Knockin Mice for Genome Editing and Cancer Modeling,” Cell 159:440-455 (2014), which is hereby incorporated by reference in its entirety). A novel bi-functional lentivirus system (HUSEC) expressing Cre-recombinase is used to initiate tumors in KPC9 by intratracheal infection. The same system enables: 1) Constitutive expression of an sgRNA against Keap1 or control to obtain KPK or KP tumors; 2) Doxycycline inducible sgRNA against Slc1a5 or control allowing for Slc1a5 LOF at initiation or progression (FIG. 20).

Doxycycline is administered to animals the day of initiation or once tumors are established (˜10 weeks post-infection), to assess the role of Slc1a5 in tumor initiation as compared to tumor progression/maintenance, respectively. Tumor growth kinetics are monitored monthly by longitudinal in vivo micro-CT imaging starting 8 weeks post-infection (determined by previous studies). To characterize the effects of CRISPR/Cas9-mediated editing of Slc1a5 on tumor initiation and progression, animals are sacrificed at 16 weeks post-infection. At least 3 tumors are micro-dissected from each animal and divided into three pieces for: 1) Fixation for H&E staining and IHC analyses to assess the levels of Slc1a5; 2) GFP-based flow cytometry to isolate tumor cells for DNA extraction and sequencing based validation (Illumina MiSeq) of mutations in targeted loci (i.e. Keap1 and Slc1a5); 3) Tumor tissue for other assays. The remaining lung lobes are collected for histopathological analysis of tumor burden, grade, and IHC staining (FIG. 20). The effects of each sgRNA on different hallmarks of tumor progression are determined. In both KPK but not in KP animals, CRISPR/Cas9-mediated Slc1a5 loss is expected to attenuate tumor growth leading to statistically significant changes in tumor burden, grade and/or the levels of markers of proliferation and cell viability.

### Assessment of the Therapeutic Potential of Slc1a5 Inhibition in Human NSCLC

The evaluations described above are used to establish the function of Slc1a5 in a KEAP1 mutant GEMM of NSCLC. In order to further assess the therapeutic potential of SLC1A5 loss in human NSCLC, the synthetic lethal interactions of SLC1A5 loss in KRAS; KEAP1 mutant cell lines and PDX models are functionally characterized.

Initially, a panel of KRAS mutant human cell lines that are WT (H2009, H2030, H1944) or KEAP1 mutant (A549 and H23) are used to perform CRISPR/Cas9-based LOF of SLC1A5 using an inducible CRISPR/Cas9 lentiviral system (HOTCG) (Aubrey et al., “An Inducible Lentiviral Guide RNA Platform Enables the Identification of Tumor-Essential Genes and Tumor-Promoting Mutations In Vivo,” Cell Reports 10:1422-1432 (2015), which is hereby incorporated by reference in its entirety). HOTCG infected GFP-positive cells are transplanted subcutaneously in immune-deficient animals and once tumors are formed, doxycycline is administered to the animals to induce the CRISPR/Cas9-based LOF of SLC1A5. This permits the effects of LOF of SLC1A5 on the growth of subcutaneous tumors to be determined. When animals are moribund, they are sacrificed and the tumor tissue is collected to assess different hallmarks of tumor progression and validate the LOF mutations in SLC1A5 (APPLICATION FIG. 20). Loss of SLC1A5 repressing the growth of KEAP1 mutant but not WT cells, validates the GEMM studies.

To further validate the human cell lines findings, PDX models are utilized, which are a valuable method to model the clinical diversity of lung cancer in mice, and are a potential resource in personalized medicine as they largely reflect the heterogeneity of a primary human tumor (Crystal et al., “Patient-derived Models of Acquired Resistance Can Identify Effective Drug Combinations for Cancer,” Science 346:1480-1486 (2014), which is hereby incorporated by reference in its entirety). NSCLC PDX models are developed, using primary resected material. In addition, PDX models are obtained from Champions Oncology who provide genetically characterized PDX models with clinical documentation of the stage, grade and histology of the primary tumors used to derive the PDX. The HOTCG lentiviral system is introduced in PDX cells prior to transplantation using previously established methods (Crystal et al., “Patient-derived Models of Acquired Resistance Can Identify Effective Drug Combinations for Cancer,” Science 346:1480-1486 (2014), which is hereby incorporated by reference in its entirety). The workflow for assessing the effects of SLC1A5 on tumor growth is similar to the experiments described above for the human cell lines (FIG. 20).

In parallel to the functional studies, the levels of SLC1A5 are evaluated by IHC in both KEAP1 mutant and WT KRAS-driven NSCLC tumors from the NYU center for Biospecimen Research. The NYU center for Biospecimen Research has a large bank of lung adenocarcinoma specimens, which were provided by Dr. Pass. All the specimens have both histopathological and clinical annotation of all patients, including date of death and recurrence. In addition, there is an assessment of morphological evaluation of the samples that can provide differentiation and prognostic information. Through a collaborative effort with Dr. Moreira and Dr. Pass targeted exome sequencing of ˜200 KRAS mutant lung adenocarcinoma biospecimens in the NYU tissue bank was recently performed to determine the mutation status of the 50 most frequently mutated genes in lung adenocarcinoma (based on TCGA). This provides a genetic sub-classification of the lung adenocarcinomas collected at NYU, which includes a significant portion of KRAS mutant lung adenocarcinomas with KEAP1 mutations (˜20%). The protein levels of SLC1A5 are then determined in the tumor bank by immunostaining, using a previously validated antibody (Hassanein et al., “SLC1A5 Mediates Glutamine Transport Required for Lung Cancer Cell Growth and Survival,” Clinical Cancer Research: an Official Journal of the American Association for Cancer Research 19:560-570 (2013), which is hereby incorporated by reference in its entirety). SLC1A5 staining status (e.g. positive, mixed or negative) and intensity are then correlated with genetic, histopathological and clinical parameters. In parallel, similar analyses are performed in KP and KPK animals generated above to confirm the histopathological correlates seen in human KRAS; KEAP1 mutant tumors are consistent in the GEMM.

### Identification of the Mechanisms of Action of Slc1a5 in the Keap1 Mutant NSCLC

The above results and analyses confirm SLC1A5 is essential for KEAP1 mutant tumors. SLC1A5 is a neutral amino acid transporter responsible for importing serine and glutamine from the extracellular environment. Preliminary results suggest that Keap1 mutant cells are dependent on the uptake of serine and glutamine to grow. These two amino acids are critical for various biosynthetic pathways. To assess the role of SLC1A5 in regulating metabolism during lung tumorigenesis, how glutamine and serine are metabolized in GEMMS and PDXs upon LOF of SLC1A5 in the context of keap1 mutant or WT models is determined.

To gain a comprehensive picture of how lung tumors utilize nutrients in an endogenous physiological context, a constant infusion of stable-isotope labeled tracers have been applied in KP lung tumors in vivo (Davidson et al., “Environment Impacts the Metabolic Dependencies of Ras-Driven Non-Small Cell Lung Cancer,” Cell Metabolism (2016), which is hereby incorporated by reference in its entirety). This approach, coupled with mass-spectrometry, enables investigation of nutrient fluxes for metabolites such as serine and glutamine. Several studies have recently described major differences between in vivo and in vitro cellular metabolism in lung cancer, highlighting the importance of performing metabolic analyses in vivo in the context of the autochthonous environment (Davidson et al., “Environment Impacts the Metabolic Dependencies of Ras-Driven Non-Small Cell Lung Cancer,” Cell Metabolism 23:517-528 (2016); Marin-Valencia et al., “Analysis of Tumor Metabolism Reveals Mitochondrial Glucose Oxidation in Genetically Diverse Human Glioblastomas in the Mouse Brain In Vivo,” Cell Metabolism 15:827-837 (2012), each of which is hereby incorporated by reference in its entirety).

SLC1A5 loss appears to be acting through import of serine and glutamine. To determine why Keap1 mutant cells require uptake of Serine and Glutamine as opposed to Keap1 WT cells, metabolic tracing of isotope-labeled glucose, glutamine and serine is performed using the in vivo models described above. Tumor bearing animals are infused with isotope-labeled glutamine or serine through the jugular vein. After 7 hours of infusion, at least 3 tumors are collected by micro-dissection and divided into three pieces for: 1) DNA extraction; 2) Fixation for H&E staining and IHC; 3) Metabolite extraction and mass-spectrometry based analysis of metabolite tracing.

The LOF status of S1c1a5 is verified in tumors by IHC and DNA sequencing. Tumors are processed for metabolite extraction using standard methods to isolate polar metabolites. Processed metabolite extracts are run on a liquid chromatography mass spec (LC-MS) instrument. Using this method, the contribution of the isotope-labeled metabolites to the various downstream biosynthetic pathways are quantitatively assessed. The combination of the sophisticated in vivo mouse and human model in conjunction with this new method for in vivo metabolic profiling enables the assessment of why KEAP1 mutant tumors have differential requirements for serine and glutamine. Finally, mechanistic insight into the metabolic changes upon SLC1A5 loss that lead to attenuation of KEAP1 tumor growth are obtained.

### Example 5—GPD2 Studies

### Determination of the Role of Gpd2 in Keap1 Mutant Lung Tumorigenesis

As shown in the previous studies and Examples, Gpd2 is required for the growth of Keap1 mutant lung cancer cells in vitro, which indicates that Gpd2 plays a crucial role in the maintenance of KPK tumors in vivo. To further establish whether Gpd2 is critical for Keap1 mutant lung adenocarcinoma growth and maintenance three separate but complementary in vivo based approaches are performed: 1) Orthotopic transplantation of syngeneic KP or KPK cells containing an inducible CRISPR/Cas9 editing system for Gpd2 LOF; 2) LOF of Gpd2 in autochthonous tumors at tumor initiation or progression in KP and KPK animals; 3) Characterization of the synthetic lethal interactions of GPD2 loss in human KRAS; KEAP1 models.

Following the extensive in vitro characterization of isogenic KP and KPK clones with differential Gpd2 status, their cellular growth is assessed in vivo by orthotopic intratracheal transplantation in the lungs of immunocompetent C57B6 mice. Preliminary results demonstrate that loss of Gpd2 is required for the growth of KPK cells. The requirement of Gpd2 for tumor maintenance is further assessed by transplanting cells orthotopically in the lung, allowing tumors to form and then temporally performing LOF of Gpd2 using previously validated sgRNAs in a doxycycline-inducible CRISPR/Cas9 editing system (Aubrey et al. 2015, Cell reports 10:1422-1432). All cells stably express luciferase, which allows for in vivo realtime monitoring of tumor growth by non-invasive bioluminescence imaging. When animals are moribund, lung tissue is collected for histopathological analysis of tumor burden and grade. To determine the effects of Keap1/Nrf2 status on different hallmarks of tumor progression, rates of proliferation and apoptosis are assessed by IHC staining of Ki67 and cleaved caspase 3, respectively. Demonstrating that Gpd2 is essential for the ability of KPK lung cancer cells to survive and grow in vivo, it is expected that statistically significant changes in tumor burden, grade and/or the levels of markers of proliferation and cell viability are observed, where KPK cells are unable to grow in the absence of Gpd2 as compared to no or less of an effect on growth of KP cells.

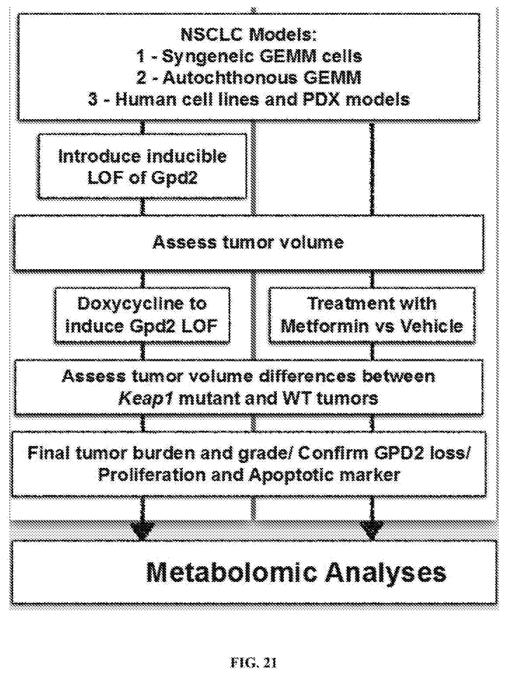

To evaluate the requirements of Gpd2 for lung tumorigenesis, LOF of Gpd2 is performed using CRISPR/Cas9-based somatic editing in KPC9 (Kras^{LSL-G12D/+}; p53^{flox/flox}; Rosa26^{LSL-Cas9-2A-GFP/LSL-CAS9-2A-GFP}) animals which express a Cre-inducible version of Cas9 as well as GFP from the ubiquitously expressed Rosa26 locus (Hasegawa et al. 2013, Exp Anim 62:295-304; Srinivas et al. 2001, BMC Dev Biol 1:4). A novel bi-functional lentivirus system (HUSEC) is used, which expresses Cre-recombinase to initiate tumors in KPC9 by intratracheal infection. The system enables: 1) Constitutive expression of an sgRNA against Keap1 or a non-targeting control to obtain KPK or KP tumors; 2) Doxycycline inducible sgRNA against Gpd2 or a non-targeting control allowing for Gpd2 LOF at tumor initiation or progression (FIG. 21).

Doxycycline is administered to animals the day of initiation or once tumors are established (˜10 weeks post-infection), to assess the role of Gpd2 in tumor initiation as compared to tumor progression/maintenance, respectively. Tumor growth kinetics are monitored monthly by longitudinal in vivo micro-CT imaging starting 8 weeks post-infection (determined by previous studies). To characterize the effects of CRISPR/Cas9-mediated editing of Gpd2 on tumor initiation and progression, animals are sacrificed at 16 weeks post-infection. At least 3 tumors are micro-dissected from each animal and divided into three for: 1) Fixation for H&E staining and IHC analyses to assess the levels of Gpd2; 2) GFP-based flow cytometry to isolate tumor cells for DNA extraction and sequencing based validation (Illumina MiSeq) of mutations in targeted loci (i.e. Keap1 and Gpd2); 3) Tumor tissue for other assays. The remaining lung lobes are collected for histopathological analysis of tumor burden, grade, and IHC staining. The effects of each sgRNA on different hallmarks of tumor progression is determined. In both KPK but not KP animals, CRISPR/Cas9-mediated Gpd2 loss is expected to attenuate tumor growth leading to statistically significant changes in tumor burden, grade and/or the levels of markers of proliferation and cell viability.

The above studies serve to establish the function of Gpd2 in a Keap1 mutant GEMM of NSCLC. In order to further evaluate the therapeutic potential of GPD2 loss in human NSCLC, the synthetic lethal interactions of GPD2 loss in KRAS; KEAP1 mutant cell lines and PDX models are characterized.

A panel of KRAS mutant human cell lines that are WT (H2009, H1944) or KEAP1 mutant (A549 and H23) are used to perform CRISPR/Cas9-based LOF of GPD2 using an inducible CRISPR/Cas9 lentiviral system (HOTCG). HOTCG infected GFP-positive cells are transplanted subcutaneously in immune-deficient animals and doxycycline is administered to the animals once tumors have formed to induce the CRISPR/Cas9-based LOF of GPD2. The effects of LOF of GPD2 on the growth of subcutaneous tumors is determined. When animals are moribund, they are sacrificed and tumors tissue collected to assess different hallmarks of tumor progression and validate the LOF mutations in GPD2 (FIG. 21). The GEMM studies are validated with loss of GPD2 repressing the growth of KEAP1 mutant but not WT cells.

To further validate the human cell lines findings, PDX models are utilized, which are a valuable method to model the clinical diversity of lung cancer in mice, and are a potential resource in personalized medicine as they largely reflect the heterogeneity of a primary human tumor. The PDX models provide genetically characterized PDX models with clinical documentation of the stage, grade and histology of the primary tumors used to derive the PDX. The HOTCG lentiviral system is introduced in PDX cells prior to transplantation using previously established methods. The workflow for assessing the effects of GPD2 on tumor growth is similar to the experiments described above for the human cell lines (FIG. 21).

### Assessment of the Prognostic and Therapeutic Potential of GPD2 Inhibition in NSCLC

The findings above serve to establish the function of Gpd2 in a Keap1 mutant human and mouse model of NSCLC. In order to assess the therapeutic potential of GPD2 loss in human NSCLC, GPD2 immunostaining is correlated with clinical features and pre-clinical studies are performed using GPD2 inhibitors.

In parallel to the functional studies, the levels of GPD2 are evaluated by IHC in both KEAP1 mutant and WT KRAS-driven NSCLC tumors from the NYU center for Biospecimen Research. The NYU center for Biospecimen Research has a large bank of lung adenocarcinoma specimens, which are provided by Dr. Harvey Pass. All the specimens have both histopathological and clinical annotation of all patients, including date of death and recurrence. In addition, there is an assessment of morphological evaluation of the samples that can provide differentiation and prognostic information. Through a collaborative effort a targeted exome sequencing of ˜200 KRAS mutant lung adenocarcinoma biospecimens in our NYU tissue bank was recently performed, to determine the mutation status of the 50 most frequently mutated genes in lung adenocarcinoma (based on TCGA). This provides a genetic sub-classification of the lung adenocarcinomas collected at NYU, which include a significant portion of KRAS mutant lung adenocarcinomas with KEAP1 mutations (˜20%). The protein levels of GPD2 in the tumor bank are determined by immunostaining, using a previously validated antibody. GPD2 staining status (e.g. positive, mixed or negative) and intensity is then correlated with genetic, histopathological and clinical parameters. Through these, the association of levels of GPD2 with worse prognosis, stage at presentation, response to chemotherapy, tumor grade, time of recurrence and overall survival is determined (i.e. multivariate Cox proportional hazards analysis). These analyses are performed with assistance from the Center for Health Informatics and Bioinformatics. In parallel, similar analyses in KP and KPK animals generated above are performed to confirm whether the histopathological correlates seen in human KRAS; KEAP1 mutant tumors are consistent in the GEMM.

The results above evaluate whether genetic loss of GPD2 can suppress tumor maintenance of KRAS; KEAP1 mutant NSCLC. In order to assess the therapeutic potential of pharmacological inhibition of GPD2 in these tumors, pre-clinical studies using the NSCLC tumor models described above are performed. Intriguingly, recent data demonstrated that the guanides/biguanides anti-diabetic drug metformin inhibits GPD2 enzymatic activity in vivo. Metformin has been previously used extensively in pre-clinical trials for various types of studies, including various cancer models, however, its activity in the context of KRAS; KEAP1 mutant NSCLC has not been previously determined.

KP, KPK GEMMs and PDX models described above are used to perform pre-clinical studies with metformin. Tumor bearing animals are randomized into vehicle and treatment arms once tumors are established. Tumor size is determined based on micro-CT imaging for GEMMs and caliper-based measurements for the subcutaneous PDX models. Intraperitoneal administration of metformin at 50 mg/kg daily over 30 days is performed, which has been shown to have on target efficacy and no toxicity. To assess the effects of metformin on tumor growth, tumor volume is monitored over the 30 days of treatment. The on-target effects of metformin on GPD2 activity are assessed by collecting tumors and performing GPD2 enzymatic activity assays. Furthermore, to characterize the effects of metformin on tumor, tumor material is collected from vehicle and metformin treated animals for: 1) IHC analyses for H&E staining, apoptotic and proliferative markers; 2) Collect tumor tissue for metabolomic assays related to Aim3. The remaining tumor material will be collected for histopathological analysis of tumor burden, grade, and IHC staining.

Metformin repression of tumor growth in KPK GEMMs and PDX models with KRAS; KEAP1 mutations, in contrast to no or marginal effects on the growth of non-KEAP1 mutant GEMMs and PDX models, confirms efficacy of a GPD2 inhibitor and these studies will have profound implications for the therapy of KRAS; KEAP1 mutant NSCLC. Metformin is already an FDA approved drug that can be further tested in clinical studies in this genetic subtype of NSCLC.

### Identification of the Mechanisms of Action of Gpd2 in Keap1 Mutant NSCLC

The results and analyses above establish whether GPD2 is essential for KEAP1 mutant tumors. GPD2 activity can have profound effects on glycolysis and oxidative phosphorylation (OXPHOS) by regulating intracellular ratios of NAD/NADH. Regulation of these metabolic processes is critical for cancer cell growth. The preliminary results indicate that Keap1 mutant cells are dependent Gpd2 for their survival. To further evaluate the role of GPD2 in regulating metabolism during lung tumorigenesis, it is determined how glucose is metabolized in: 1) GEMMs and PDXs upon LOF of GPD2 in the context of keap1 mutant or WT models described above; and 2) in the response of GPD2 inhibition of metformin described above (FIG. 21).

To gain a comprehensive picture of how lung tumors utilize nutrients in an endogenous physiological context, a constant infusion of stable-isotope labeled tracers have been applied in KP lung tumors in vivo. This approach, coupled with mass-spectrometry, enables investigation of nutrient fluxes for metabolites such as serine and glutamine. Several studies have recently described major differences between in vivo and in vitro cellular metabolism in lung cancer, highlighting the importance of performing metabolic analyses in vivo in the context of the autochthonous environment.

To determine why Keap1 mutant cells require GPD2 activity as opposed to Keap1 WT cells, metabolic tracing of isotope-labeled glucose is performed using the in vivo models described above. Similar to the prior studies, tumor bearing animals are infused with isotope-labeled glucose through the jugular vein. After 7 hours of infusion, at least 3 tumors are collected by micro-dissection and divided into three pieces for: 1) DNA extraction; 2) Fixation for H&E staining and IHC; 3) Metabolite extraction and mass-spectrometry based analysis of metabolite tracing.

The LOF status of Gpd2 in tumors is verified by IHC and DNA sequencing. Tumors are processed for metabolite extraction using standard methods to isolate polar metabolites. Processed metabolite extracts are run on a liquid chromatography mass spec (LC-MS) instrument at the Whitehead Metabolite Core Facility at MIT. Using this method, the contribution of the isotope-labeled metabolites to the various downstream biosynthetic pathways that GPD2 is known to regulate are quantitatively assessed. The rates of glycolysis and TCA cycle are then focused on. Specifically, the rates of glucose-derived lactate and TCA cycle incorporation of glucose-derived pyruvate are assessed and the ratios of NAD/NADH are analyzed. GPD2 is predicted to be essential for Keap1 mutant cells because they rely on GPD2 to maintain homeostatic levels of NAD/NADH in order to maintain glycolysis and TCA cycle. Therefore, genetic loss or pharmacological inhibition of GPD2 will lead to decreased NAD/NADH levels and a decrease in glycolysis in Keap1 mutant but not WT cells. The combination of the sophisticated in vivo mouse and human model in conjunction with this new method for in vivo metabolic profiling further enables the determination of why KEAP1 mutant tumors have differential requirements for serine and glutamine. Finally, mechanistic insight into the metabolic changes upon GPD2 loss that lead to attenuation of KEAP1 tumor growth are obtained.

### REFERENCES

Cox, A. D., Fesik, S. W., Kimmelman, A. C., Luo, J. & Der, C. J. Drugging the undruggable RAS: mission possible? Nat. Rev. Drug Discov. 13, 828-851 (2014).
Berger, A. H. et al. High-throughput phenotyping of lung cancer somatic mutations. Cancer Cell 30, 214-228 (2016).
Singh, A. et al. Dysfunctional KEAP1-NRF2 interaction in non-small-cell lung cancer. PLoS Med. 3, e420 (2006).
Itoh, K. et al. An Nrf2/small Maf heterodimer mediates the induction of phase II detoxifying enzyme genes through antioxidant response elements. Biochem. Biophys. Res. Commun. 236, 313-322 (1997).
Itoh, K. et al. Keap1 represses nuclear activation of antioxidant responsive elements by Nrf2 through binding to the amino-terminal Neh2 domain. Genes Dev. 13, 76-86 (1999).
Harris, I. S. et al. Glutathione and thioredoxin antioxidant pathways synergize to drive cancer initiation and progression. Cancer Cell 27, 211-222 (2015).
DeNicola, G. M. et al. NRF2 regulates serine biosynthesis in non-small cell lung cancer. Nat. Genet. 47, 1475-1481 (2015).
Sullivan, L. B., Gui, D. Y. & Heiden, M. G. V. Altered metabolite levels in cancer: implications for tumour biology and cancer therapy. Nat. Rev. Cancer 16, 680-693 (2016).
DuPage, M., Dooley, A. L. & Jacks, T. Conditional mouse lung cancer models using adenoviral or lentiviral delivery of Cre recombinase. Nat. Protoc. 4, 1064-1072 (2009).
Sanchez-Rivera, F. J. et al. Rapid modelling of cooperating genetic events in cancer through somatic genome editing. Nature 516, 428-431 (2014).
Maresch, R. et al. Multiplexed pancreatic genome engineering and cancer induction by transfection-based CRISPR/Cas9 delivery in mice. Nat. Commun. 7,10770 (2016).
Meylan, E. et al. Requirement for NF-κB signalling in a mouse model of lung adenocarcinoma. Nature 462, 104-107 (2009).
Altman, B. J., Stine, Z. E. & Dang, C. V. From Krebs to clinic: glutamine metabolism to cancer therapy. Nat. Rev. Cancer 16, 619-634 (2016).
Kerr, E. M., Gaude, E., Turrell, F. K., Frezza, C. & Martins, C. P. Mutant Kras copy number defines metabolic reprogramming and therapeutic susceptibilities. Nature 531, 110-113 (2016).
Jaramillo, M. C. & Zhang, D. D. The emerging role of the Nrf2-Keap1 signaling pathway in cancer. Genes Dev. 27, 2179-2191 (2013).
Konstantinopoulos, P. A. et al. Keap1 mutations and Nrf2 pathway activation in epithelial ovarian cancer. Cancer Res. 71, 5081-5089 (2011).
Shibata, T. et al. Genetic alteration of Keap1 confers constitutive Nrf2 activation and resistance to chemotherapy in gallbladder cancer. Gastroenterology 135, 1358-1368 (2008).
Kim, Y. R. et al. Oncogenic NRF2 mutations in squamous cell carcinomas of oesophagus and skin. J. Pathol. 220, 446-451 (2010).
Sato, Y. et al. Integrated molecular analysis of clear-cell renal cell carcinoma. Nat. Genet. 45, 860-867 (2013).
Fabrizio, F. P. et al. Keap1/Nrf2 pathway in kidney cancer: frequent methylation of KEAP1 gene promoter in clear renal cell carcinoma. Oncotarget 8, 11187-11198 (2017).
Muscarella, L. A. et al. Regulation of KEAP1 expression by promoter methylation in malignant gliomas and association with patient's outcome. Epigenetics 6, 317-325 (2011).
Hanada, N. et al. Methylation of the KEAP1 gene promoter region in human colorectal cancer. BMC Cancer 12, 66 (2012).
Krall, E. B. et al. KEAP1 loss modulates sensitivity to kinase targeted therapy in lung cancer. eLife 6, e18970 (2017).
Bolger, A. M., Lohse, M. & Usadel, B. Trimmomatic: a flexible trimmer for Illumina sequence data. Bioinformatics 30, 2114-2120 (2014).
Li, H. & Durbin, R. Fast and accurate short read alignment with Burrows-Wheeler transform. Bioinformatics 25, 1754-1760 (2009).
Tarasov, A., Vilella, A. J., Cuppen, E., Nijman, I. J. & Prins, P. Sambamba: fast processing of NGS alignment formats. Bioinformatics 31, 2032-2034 (2015).
DePristo, M. A. et al. A framework for variation discovery and genotyping using next-generation DNA sequencing data. Nat. Genet. 43, 491-498 (2011).
Cibulskis, K. et al. Sensitive detection of somatic point mutations in impure and heterogeneous cancer samples. Nat. Biotechnol. 31, 213-219 (2013).
Gardner, E. E. et al. Chemosensitive relapse in small cell lung cancer proceeds through an EZH2-SLFN1L axis. Cancer Cell 31, 286-299 (2017).
Cheng, D. T. et al. Memorial Sloan Kettering-Integrated Mutation Profiling of Actionable Cancer Targets (MSK-IMPACT): a hybridization capture-based next-generation sequencing clinical assay for solid tumor molecular oncology. J. Mol. Diagn. 17, 251-264 (2015).
Schneeberger, V. E., Allaj, V., Gardner, E. E., Poirier, J. T. & Rudin, C. M. Quantitation of murine stroma and selective purification of the human tumor component of patient-derived xenografts for genomic analysis. PLoS One 11, e0160587 (2016).
Li, B. & Dewey, C. N. RSEM: accurate transcript quantification from RNA-Seq data with or without a reference genome. BMC Bioinformatics 12, 323 (2011).
Li, C. M. et al. Foxa2 and Cdx2 cooperate with Nkx2-1 to inhibit lung adenocarcinoma metastasis. Genes Dev. 29, 1850-1862 (2015).
Biton, A. et al. Independent component analysis uncovers the landscape of the bladder tumor transcriptome and reveals insights into luminal and basal subtypes. Cell Rep. 9, 1235-1245 (2014).
Lewis, C. A. et al. Tracing compartmentalized NADPH metabolism in the cytosol and mitochondria of mammalian cells. Mol. Cell 55, 253-263 (2014).
Young, J. D., Walther, J. L., Antoniewicz, M. R., Yoo, H. & Stephanopoulos, G. An elementary metabolite unit (EMU) based method of isotopically nonstationary flux analysis. Biotechnol. Bioeng. 99, 686-699 (2008).
Fernandez, C. A., Des Rosiers, C., Previs, S. F., David, F. & Brunengraber, H. Correction of ¹³C mass isotopomer distributions for natural stable isotope abundance. J. Mass Spectrom. 31, 255-262 (1996).

### Example 6

### The Role of Metabolic Rewiring in Lung Cancer Immune Surveillance

Lung cancer is the leading cause of cancer-related deaths in the United States and worldwide¹⁴. A major subtype of lung cancer is NSCLC, which accounts for ˜85% of all lung cancer cases. Approximately 30% of human NSCLC tumors acquire GOF mutations in the anti-oxidant transcription factor NRF2 or LOF mutations in KEAP1, the negative regulator of NRF2. In KRAS-driven lung adenocarcinomas, a subtype of NSCLC, approximately 20% of tumors contain KEAP1 mutations. The high mutation frequency of the NRF2/KEAP1 pathway suggests an important role for oxidative stress homeostasis in maintaining cancer cell survival during lung carcinogenesis. This presented an opportunity to identify novel therapeutic strategies for patients with this genetic subtype (NRF2/KEAP1 mutant) of NSCLC.

Genetically engineered mouse models (GEMMs) of lung cancer faithfully mimic human lung tumors in their progression, showing similarities both at the molecular and histopathological levels and have greatly assisted the study of human lung cancers^{15,16}. In these models, lung tumors are induced by the conditional activation of the oncogene Kras^{G12D} and deletion of the tumor suppressor p53 (K-ras^{LSL-G12D/+}; p53^{flox/flox}, hereafter KP) in the lung via intratracheal administration of viral vectors expressing Cre-recombinase. Prior studies using these GEMMs have been largely hindered by the lack of tractable in vivo experimental systems to study genes often co-mutated with KRAS, including the KEAP1/NRF2 pathway^{17,18}; therefore, the tumor cell autonomous and non-cell autonomous role of Keap1/Nrf2 during tumorigenesis remained unclear.

The role of NRF2 in antioxidant production and metabolic rewiring: Tumorigenesis requires cancer cells to increase their metabolic output to support tumor growth. Higher metabolic activity increases oxidative stress in the form of reactive oxygen species (ROS), which can lead to oxidative damage of macromolecules. Emerging evidence from our lab and others supports the idea that cancers increase antioxidant capacity as a stress response mechanism, suggesting that high ROS levels may constitute a barrier to tumorigenesis^{1,2,18}. The anti-oxidant transcription factor NRF2 acts as the primary line of defense against ROS to maintain oxidative homeostasis by regulating the expression of a plethora of genes involved in ROS clearance and rewiring of cellular metabolism¹⁹.

We have demonstrated, including in the above examples and studies, that activation of NRF2, in either mouse or human cancer cells, leads to increased dependency on exogenous amino acids (e.g. Serine, Glutamine), which become limiting for the tricarboxylic acid cycle and other biosynthetic reactions creating a metabolic bottleneck. Cancers with genetic or pharmacological activation of the NRF2 antioxidant pathway have a metabolic imbalance between supporting increased antioxidant capacity over central carbon metabolism, which can be therapeutically exploited^{1,2}.

Increased metabolism of rapidly proliferating tumor cells is an intriguing target for novel therapeutics against cancer. Having previously established that the heightened metabolism of malignant cells represents a cell intrinsic vulnerability in a subtype of lung cancer, the present studies will further evaluate whether in addition to changes within the tumor cells themselves, the precipitous nutrient utilization by the tumor also starves off the immune response, weakening anti-tumor immune responses. The present studies explore the impact of heightened tumor metabolism on the immune responses and examine combination therapy that simultaneously targets tumor metabolism and boosts immune responses as a viable strategy for some types of lung cancer.

KRAS driven lung adenocarcinoma (LUAD) remains a major unmet clinical challenge in oncology, despite the advent of cancer immunotherapy. One approach to targeting these difficult to treat malignancies is to target the high bioenergetic demands of the rapidly proliferating malignant cells. The KEAP1/NRF2 antioxidant pathway is mutated in ˜20% KRAS-driven NSCLC and therefore presents a major therapeutic target. The prior examples and studies establish that the loss of Keap1 by the tumor cells leads to hyperactivation of the NRF2 pathway, increased glutamine metabolism and changes in the tumor microenvironment. The present studies aim to further evaluate how changes in tumor metabolism, and in particular how elevated glutaminolysis of tumor cells with augmented NRF2 pathway, influence the immune responses to cancer. Having demonstrated that competition for glutamine dampens anti-tumor T cell response, we now explore how competition for this key nutrient impacts CD8+ T cell differentiation, activation, proliferation and cytokine production, as well as recruitment to the tumor site. We take advantage of our autochthonous animal models of LUAD, well established in vitro assays and stratified patient biospecimens to further examine how tumor genotype-specific changes in metabolism influence T cell responses.

We hypothesize that activation of the NRF2 pathway by the tumor cells leads not only to cell intrinsic changes within the malignant cells, but also impacts the tumor microenvironment through take up of key nutrients. Increased glutaminolysis of the Keap1 mutant cells represent a key vulnerability for these tumor cells. These studies build on our earlier observation that hyperactivation of the NRF2 pathway leaves the malignant cells uniquely sensitive to glutamine deprivation and further examine the therapeutic potential of combination therapy of check-point blockade and glutaminase inhibitor.

Using both genetically defined mouse models and patient tumors, these studies further aim to identify novel precision medicine approaches to simultaneously target tumor metabolism and enhance immune responses. Further, the present studies may uncover therapeutic vulnerability in other cancers with genetic, epigenetic or post-transcriptional alterations in the KEAP1/NRF2 pathway, paving way for development of immune-based therapeutic interventions.

Immune-based therapies for NSCLC have yielded limited responses in KRAS-mutant patients, limited by low overall response rates. Emerging evidence suggests that metabolic competition of tumor cells and adaptive immune cells can modulate immune-based anti-tumor responses. What remains unclear is how genetic subtypes of KRAS-mutant tumors impact the metabolic microenvironment, leading to immune evasion. Approximately 20% of KRAS-mutant LUAD tumors carry loss-of-function mutations in KEAP1, a negative regulator of NRF2, which is the master transcriptional regulator of the antioxidant response. The above studies and examples, including published results based thereon, demonstrate that loss of Keap1 activates Nrf2, dramatically rewires cellular metabolism, accelerates KRAS-driven LUAD and renders tumors dependent on glutamine metabolism^{1,2}. In addition to identifying tumor-specific druggable metabolic vulnerabilities (e.g. glutamine), based on our data and existing in silico analyses of patient tumors³, we propose that the substantial rewiring of metabolism in KEAP1 mutant tumors impacts the metabolic milieu in the tumor microenvironment in a manner that facilitates tumor immune evasion. The present studies will further elucidate how changes in the NRF2/KEAP1 pathway within the malignant cells influence the tumor microenvironment and subvert the immune response to cancer. We further examine the clinical potential of combining check-point blockade with glutaminase inhibitor to treat Keap1 mutant subtype of LUAD. The studies aim to identify tumor vulnerabilities that can be used for precision medicine approaches to target the specific genetic subtypes of lung cancer. We believe that our studies and results are relevant to identifying tumor vulnerabilities in other cancers with genetic⁴⁻⁹, epigenetic¹⁰⁻¹², or post-transcriptional¹³ alterations in the KEAP1/NRF2 pathway.

The following are aims in accordance with these studies:

Aim1: Characterize the T Cell Infiltration, Activation and Cytokine Production in Genetic Subtypes of LUAD.

We evaluate T cell recruitment, activation, and localization in the context of lung adenocarcinoma tumors both from patients stratified along KEAP1/NRF2 pathway mutations and from our KRAS-driven GEMM in the presence and absence of the Keap1 mutation.

Aim2: Define and Functionally Characterize the Metabolic Pathways that Suppress T Cell Function in Keap1 Mutant Tumors.

We leverage our expertise in CRISPR/Cas9-based and metabolomics both in vitro and in vivo systems to determine how metabolites consumed and secreted by the cancer cells influence T cell differentiation, activation and proliferation. Finally, we perform unbiased gain-of-function genetic screens (positive selection) to identify novel metabolic genes/pathways that lead to immune evasion by abrogating CD8 T cell killing.

Aim3: Assess the Efficacy of Combining Immunotherapy with Metabolic Inhibitors to Treat Keap1 Lung Adenocarcinoma.

Our studies predict that immune evasion as a consequence of increased metabolite uptake by KEAP1 mutant tumors renders them refractory to immunotherapy. Using a novel KRAS-driven GEMM that expresses the checkpoint inhibitor PDL1, we test whether combination therapy of a potent immune anti-PDL1 checkpoint inhibitor with: A) a glutaminase inhibitor (phase I clinical trials) that specifically targets KEAP1 mutant tumors, or B) genetic or pharmacologic inhibition of other metabolic pathways (provided in Aim1), leads to robust anti-tumor immune responses as compared to checkpoint inhibition alone.

Immune responses against cancer are primarily guided by T lymphocytes that recognize protein based antigens in the context of major histocompatibility complex on the surface of other cells. The CD4+ helper T cells and the CD8+ cytotoxic T cells (CTLs) orchestrate the humoral and cell-mediated anti-tumor responses through the production of cytokines, perforin and granzymes²⁰. A major immune-based therapeutic approach targeting immune-regulatory membrane receptors to enhance T cell effector function has demonstrated some efficacy in a number of solid malignancies including NSCLC²¹.

Engagement of co-inhibitory immune checkpoint receptors that include cytotoxic T-lymphocyte-associated protein 4 (CTLA4), programmed cell death 1 (PD1) or other checkpoint receptors²² following recognition of MHC-antigen complex by the TCR modulates the response of T lymphocytes. While these checkpoints play a critical role in the maintenance of self-tolerance and in the regulation of the amplitude and duration of T cell response, in the context of anti-cancer responses they often are incriminated in the blunting of the response as the ligands of CTLA4 and PD1 are often upregulated in the tumor microenvironment. Immunotherapy approaches leverage the patient's immune system to induce a cellular and humoral response against malignant cells by unleashing the full potential of the immune response against cancer. The main modalities of cancer immunotherapy in NSCLC focus on CTLA4 and PD-L1/PD-1 pathway. While therapies targeting immune checkpoints have shown promise in the context of NSCLC and combination therapies of checkpoint inhibitors with other therapeutic modalities are currently being explored, the overall response rate to immune modulators in the context of lung cancer has remained at 30% or less²³.

Effector T cells have distinct metabolic requirements that enable their proper activation and differentiation in order to function²⁴. It is becoming increasingly clear that nutrient uptake and utilization in T cells is critical for the control of their differentiation, function and survival. Interestingly, the nutrients that are essential for T cell activation and differentiation are also greatly consumed and utilized by tumors. The best examples of such nutrients are glucose and glutamine, which are required for both tumor cells and T cell proliferation²⁵⁻²⁷. Emerging evidence about the competition for metabolites between tumor cells and T cells or the secretion of immunosuppressive metabolites in the tumor microenvironment points to a potential role for this metabolic competition contributing to immune evasion. However, it has remained unknown whether mutations (e.g. KEAP1, LKB1) that lead to cell autonomous tumor cell metabolic reprogramming alter the metabolic milieu in the tumor microenvironment, which can limit nutrient availability for cytotoxic CD8 T cells.

Functional Characterization of KEAP1 Mutations in KRAS-Driven Lung Cancer.

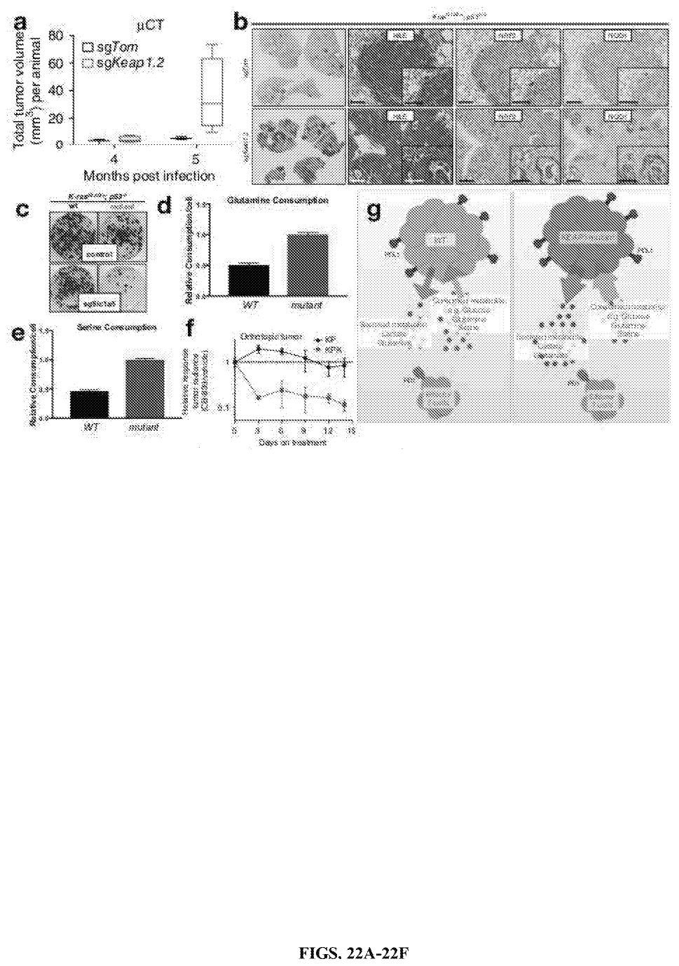

We previously developed a novel CRISPR/Cas9-based in vivo genome engineering method that allows us to rapidly, efficiently, and systematically interrogate the functional role of genes in this Kras-driven GEMM of NSCLC²⁸. Using this approach, we demonstrated that CRISPR/Cas9-based somatic loss of the negative regulator of Nrf2, Keap1, strongly promotes NSCLC progression (see above Examples and FIG. 22a-b). Furthermore, we demonstrated that KEAP1 mutations in human patients are predictors of more advanced LUAD with poorer clinical outcomes. Having established the importance of KEAP1 mutations in mouse KRAS-driven LUAD, we evaluated potential therapeutic vulnerabilities in this genetic subtype of lung cancer. To identify genotype specific vulnerabilities in KEAP1 mutant KRAS-driven NSCLC, we performed a focused CRISPR/Cas9-based genetic screen and identified that loss of Slc1a5, a neutral amino acid transporter of serine and glutamine, is synthetic lethal in KEAP1 mutant but not wild-type GEMM-derived lung cancer cell lines (FIG. 14B and FIG. 22c)¹. Recent studies have demonstrated that high levels of SCL1A5 in NSCLC tumors correlate with poor survival^{29,30}. Furthermore, when we examined glutamine utilization by the KPK and KP isogenic lines, we observed significantly increased glutamine and serine utilization by KPK cells (FIG. 22d-e). Critically, KPK cells were far more sensitive than KP cells to CB-839, a selective glutaminase inhibitor, a rate limiting enzyme in glutamine metabolism (FIG. 10 and FIG. 22f)^{1,2}.

Impact of Altered Glutamine Metabolism on T Lymphocytes in Keap1 Mutant Lung Adenocarcinoma.

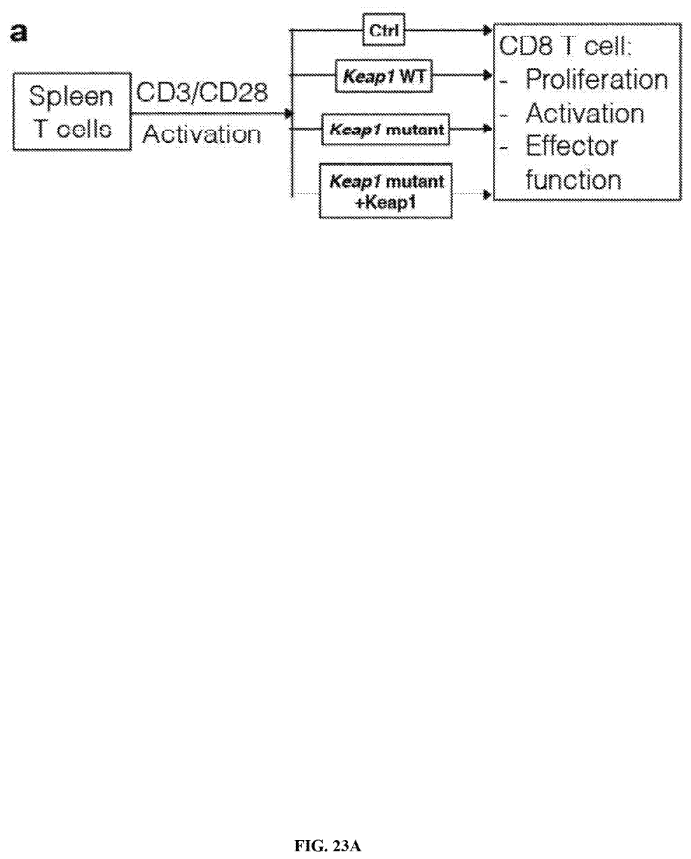

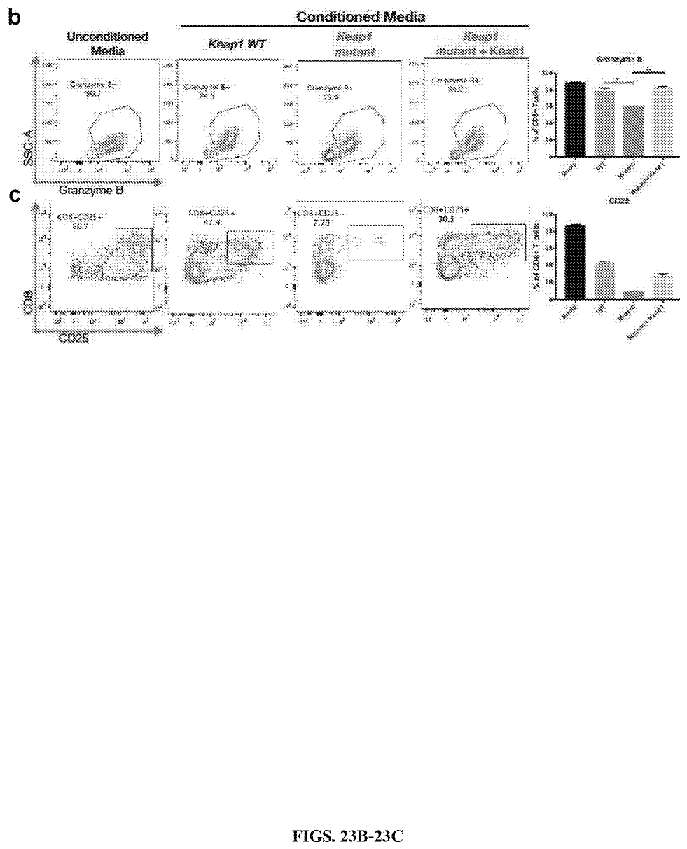

Effective immune response to cancer relies on killing of the cancer cells by cytotoxic T lymphocytes. Immune suppression by the tumor microenvironment can have a profound impact on immune surveillance. Indeed, exclusion of cytotoxic CD8+ T lymphocytes (CTLs) from the vicinity of cancer is likely one of the primary immune evasion strategies of solid malignancies as has been originally demonstrated for ovarian cancer and pancreatic duct adenocarcinoma^{31,32}. We examined whether direct competition for metabolic resources represented a relevant mode of immune evasion by the malignant cells in LUAD and if tumors that upregulate the Nrf2 pathway dampen immune responses through competing for nutrients with the infiltrating immune cells (FIG. 22g). We first examined whether CTL activated in presence of supernatant from KPK cells vs. KP isogenic cells responded differently to stimuli (FIG. 23a). Strikingly, we observed that CTL activated in KPK conditioned media failed to upregulate markers of activation CD25 and produced significantly less of IFNγ or the effector protease, granzyme-B, compared to CD8+ T cells grown in media from KP tumor cells (FIG. 23b). These results indicate that tumor-intrinsic mutations in Keap1 in LUAD impact T cell activation, proliferation and function.

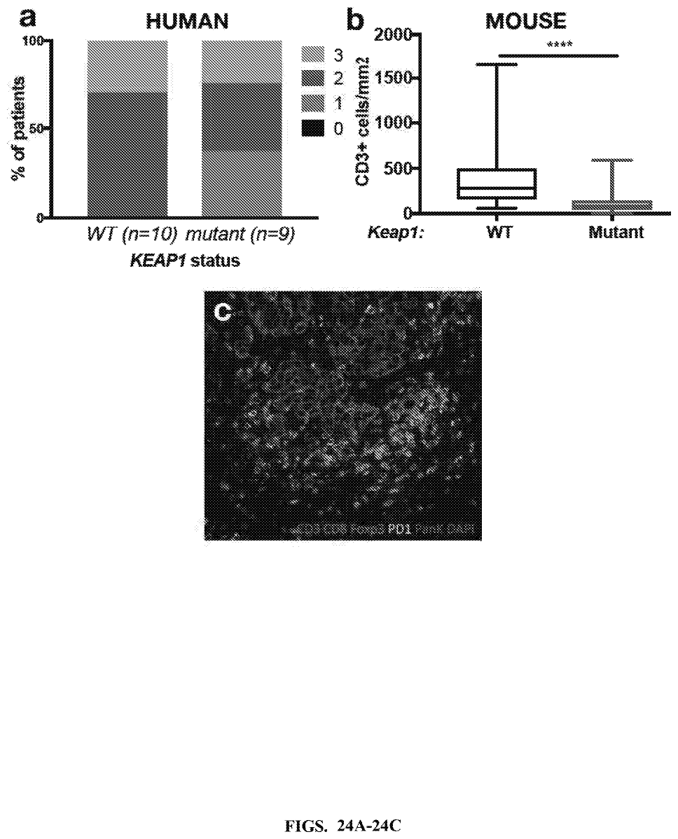

To examine whether altered tumor metabolism as a consequence of KEAP1 mutation has an impact on immune surveillance in vivo, we first examined whether patients with KRAS-driven LUAD that carry a KEAP1 mutation have any appreciable change in T cell infiltration. Patient samples from the NYU Biospecimen Repository were stratified based on mutation analysis of KEAP1 and activation of Nrf2 pathway confirmed through staining of target gene NQO1¹. In a first set of experiments, histological characterization of CD3+ infiltration in stained slides from LUAD samples of patients with and without a Keap1 mutation revealed that altered Nrf2 signaling correlates with markedly decreased T cell infiltration (FIG. 24a). Furthermore, analysis of our KP and KPK GEMMS revealed a similar trend (FIG. 24b). Taken together, these results indicate that tumor intrinsic glutamine metabolism influences T cell mediated immune surveillance by altering the tumor microenvironment.

### EXPERIMENTAL DESIGN

Aim1: Characterize the T Cell Infiltration, Activation and Cytokine Production in Genetic Subtypes of LUAD.

Having previously established that loss of KEAP1 leads to hyperactivation of the NRF2 antioxidant pathway and accelerates KRAS-driven lung tumorigenesis in a tumor cell intrinsic manner we further examine how altered metabolism of tumor cells influences immune responses to cancer. Our initial data reveals that genotype-specific changes in the NRF2 pathway of cancer cells can influence the metabolic landscape of the tumor microenvironment. In particular, changes in glutamine and serine uptake by lung cancer cells results in significant changes in T cell recruitment to the tumor, and in cytotoxic T cell activation and proliferation in vitro. We leverage our combined expertise in immunology, cancer biology, and metabolomics to evaluate how elevated glutaminolysis of tumor cells with augmented NRF2 pathway influence T cell differentiation in a tumor-genotype-specific manner.

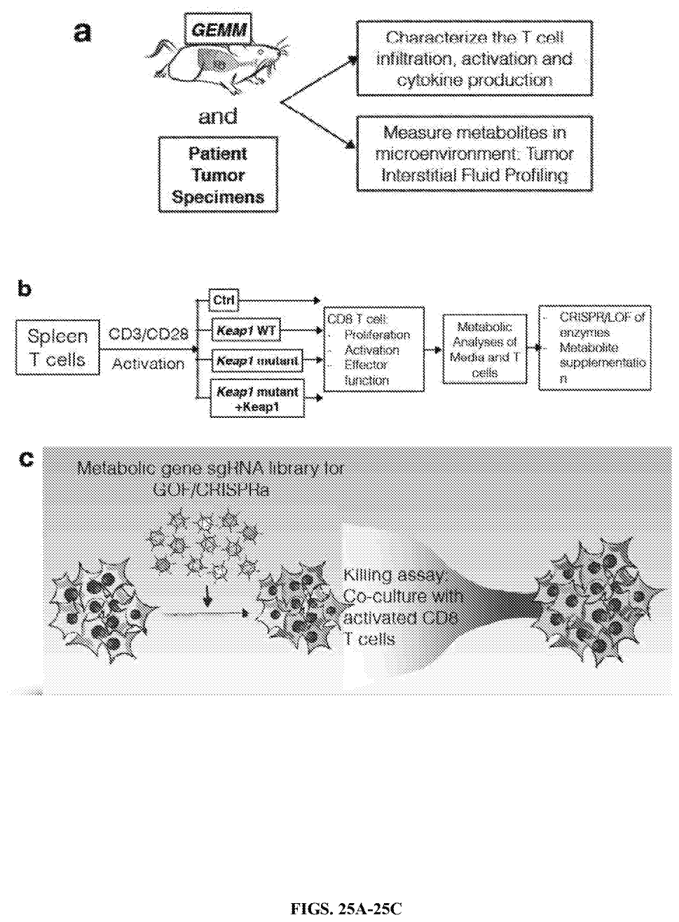

Initially, T cell infiltration, activation and cytokine production is characterized in Keap1 WT and mutant tumors from patient biospecimens and in our GEMMS. Both the autochthonous KRAS-driven KP and KPK model³³, and orthotopic intratracheal transplants of syngeneic KP and KPK cell lines in C57B6/J animals are utilized (FIG. 25a). These animal models are well established and characterized in our laboratory, including as above described^{1,2}. Detailed FACS analysis of the leukocytes in the lungs of the animals is performed to define lymphocytic infiltrate, maturation and activation status of the lymphocytes, chemokine receptor expression and presence of the innate cells that the T cells recruit. Intracellular FACS analysis is conducted to characterize the cytokines that the Th and CTL are secreting (IFNγ, IL-2, IL-4, IL-10, IL-17, IL-22) as well as to evaluate perforin and granzyme B production by CTLs. Furthermore, immunohistochemical and multicolor immunofluorescence (IF) analysis is conducted to evaluate the migration of the T cells and innate cells relative to the tumors (FIG. 24c). To distinguish Keap1 sufficient and deficient tumors in our histology sections Nqo1, a previously characterized target of the Nrf2 pathway in both mouse and human tumors, is stained for (FIG. 22b)′.

FACS and histological analysis is conducted on biospecimens from tumor biopsies of patients with KRAS driven LUAD. Tumor samples are genotyped for Keap1 mutations and immunohistochemical staining for NQO1 to further validate our categorization of patient samples. For histological evaluations, tumor size is evaluated and the immune infiltrate is characterized. As ˜20% of LUAD patients have inactivation of Keap1 we expect to compare T cell infiltration and activation following genotypic stratification of the patient samples for NRF2 activation. Finally, 10× single-cell RNA sequencing of T cell populations from both mouse and human CD8 T cells is performed in order to obtain a more comprehensive transcriptome-based map of the T cell populations with KEAP1 WT and mutant tumors.

Aim2: Define and Functionally Characterize the Metabolic Pathways that Suppress T Cell Function in Keap1 Mutant Tumors.

Identification of Genotype-Specific Metabolic Alterations in the Tumor Microenvironment.

The preliminary focus of this analysis is on the elevated glutamine and serine of Keap1 mutant LUAD and how increased glutamine uptake by the tumors impacts T cell mediated immune surveillance. This focus is based on prior work, including as provided in the above examples, that identified increased dependence of Keap1 tumors on glutamine and serine as a cell intrinsic vulnerability. To determine the levels of metabolites in the tumor microenvironment lung tumors from KP and KPK animals and the tumor interstitial fluid (TIF)³⁴ is isolated and profiled by mass-spec analysis using gas-chromatography mass spectrometry (GC-MS) and/or liquid-chromatography mass spectrometry (LC-MS) (FIG. 25a). Furthermore, the differential levels of the TIF metabolites are compared with what the levels observed in KP and KPK cells condition media in vitro.

Assess the Function of Genotype-Specific Metabolic Changes on T Cell Function In Vitro.

In order to establish how altered metabolism of KPK as compared to KP LUAD tumors impacts CTL differentiation, function and proliferation in vitro assays are performed using standard T cell activation assays in the presence of conditioned medium with and without supplementation of glutamine or serine (or other metabolites) (FIG. 25b). Proliferation, activation, cytokine production and production of cytolytic proteins, perforin and granzyme B is examined by FACS. Furthermore, upon observing that supplementation of serine or glutamine (or other metabolites) rescues T cell activation/proliferation, stable-isotope metabolite tracing is performed to characterize the utilization of supplemented glutamine or serine by using ¹³C-labeled glutamine, serine or other tracers depending on the identified metabolite in the T cells². This further enables the identification of downstream metabolites that utilize carbons from glutamine and/or serine. Finally, we predict that the tracing will further indicate specific metabolites and metabolic enzymes that are required for T cell proliferation and activation. We perform: 1) CRISPR/Cas9-based LOF of the various metabolic enzymes, and 2) rescue experiment by directly supplementing the metabolites (if cell permeable forms available) that are generated by these metabolic enzymes, to further assess their impact on T cell proliferation and function using in vitro assays (FIG. 25b).

Unbiased Identification of Metabolic Modulators of Immune Surveillance in Keap1 Mutant LUAD.

An unbiased genetic screen is used to identify metabolic mediators of immune evasion. We leverage our expertise in CRISPR/Cas9-transcriptional activator genetic screen to perform a gain-of-function positive selection screen to identify metabolic genes whose increased expression can lead to immune evasion by suppressing CD8 T cell mediated killing. Briefly, we have generated a novel library of sgRNAs targeting promoters of 3000 metabolic genes (3000 metabolic genes×10 sgRNAs per gene and 100 control sgRNAs)^{35,36} (FIG. 25c). We predict positive selection and outgrowth of cells that express sgRNAs that cause the transcriptional activation/overexpression of metabolic genes that promote the increased consumption or secretion of metabolites that can suppress CD8 T cell killing. A positive control sgRNA is included that induces expression of the PDL1 checkpoint inhibitor (sgPDL1—see Aim3 below), which is able to suppress CD8 T cell killing. Candidate genes identified from the screen are validated individually to demonstrate that GOF can lead to suppression of T cell killing. Furthermore, LOF of candidate genes is performed to validate that loss of these metabolic genes can promote CD8 T cell killing in vitro. Our focus on CD8+ T lymphocytes is justified based on preliminary analysis and their importance in current immune-based anti-tumor therapies. Nonetheless, we also assess the impact of the metabolic competition between KP/KPK tumors and other immune populations (e.g. Th cells, NKT and NK cells).

Aim3: Assess the Efficacy of Combining Immunotherapy with Metabolic Inhibitors to Treat Keap1 Lung Adenocarcinoma.

Characterize the T Cell Infiltration in Response to Suppression of Keap1 Mutant Tumor Metabolism.

Previous studies including as described in the above examples identified glutamine dependence as a key metabolic vulnerability of KRAS-driven LUAD tumors with mutations in the Nrf2 pathway. As such, these tumors are sensitive to CB839, a glutaminase inhibitor, currently in phase I clinical trials in NSCLC. Importantly, CB839 does not negatively impact T cell function (data not shown). Furthermore, based findings from Aim1, other candidate metabolites, including serine, are evaluated/identified which are highly consumed or secreted in Keap1 mutant tumors and which have functional impact on CD8 T cell proliferation/activation in vitro. We take advantage of our mouse model and treat KPK and KP tumors with: 1) CB839 or 2) use a genetic approach with doxycycline inducible hairpins to knockdown the transporters or the rate-limiting metabolic enzymes in the Keap1 mutant tumors, in order to further assess whether in addition to impacting tumor growth in a cell intrinsic manner, infiltration, proliferation and cytokine production of CD8+ T cells is altered in response to CB839 or knockdown of the other metabolic enzymes. We expect enhanced T cell infiltration and activation in Keap1 mutant tumors upon treatment of animals with CB839.

Assess the Therapeutic Potential of Combining Checkpoint Inhibitors in Combination with Suppression of Keap1 Mutant Tumor Metabolism Using a Novel GEMM.

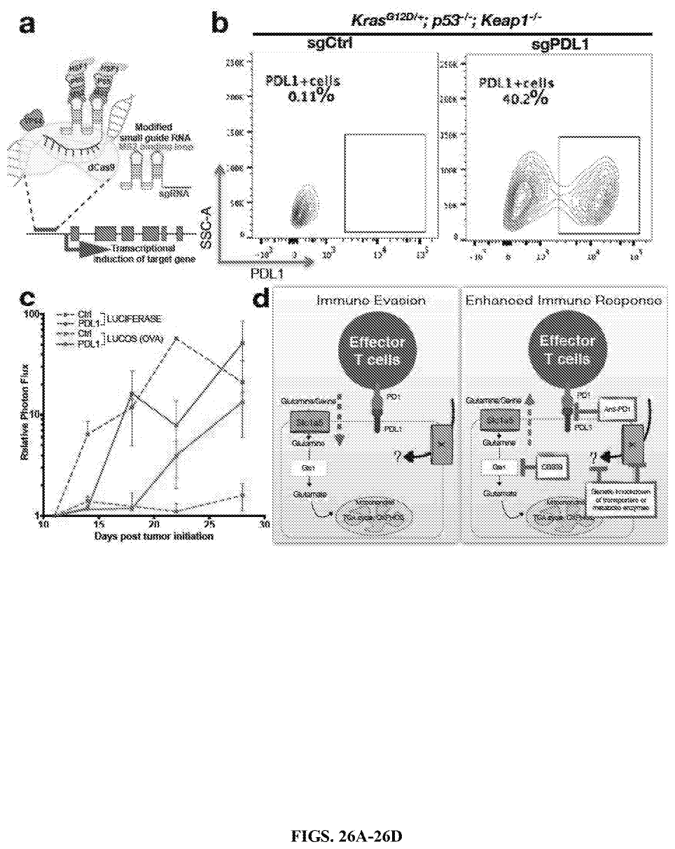

Immune checkpoint blockade has shown efficacy in a number of solid malignancies through enhancement of T cell responses, however the results in LUAD have been underwhelming²¹. Since T cell activation and proliferation are metabolically demanding processes, we further examine whether combining checkpoint inhibitor therapy with a glutaminase inhibitor, such as CB839, or suppression of other metabolic enzymes/pathways is a viable alternative for enhancement of anti-tumor immunity. Because murine KP tumors do not express high level of T cell immunomodulatory ligands, we have used CRISPR/Cas9-based transcriptional activator approaches³⁷ to engineer KP and KPK cells that have increased endogenous expression of PDL1, leading to high surface expression of the inhibitory co-receptor thought to be most relevant to LUAD (FIG. 26a-b). Using these engineered cells, we examine the efficacy of treating animals with KP and KPK PDL1 positive tumors with either anti-PD1 blockade, CB839 alone, or with combination therapy and assess the rate of tumor growth (non-invasive bioluminescence imaging and micro-CT), mouse survival (Kaplan-Meier), T cell proliferation and activation, T cell localization within the tumors and development of tertiary lymphoid structures within the diseased lungs (FIG. 26c-d)³⁸.

### REFERENCES

1 Romero, R. et al. Keap1 loss promotes Kras-driven lung cancer and results in dependence on glutaminolysis. Nat Med 23, 1362-1368, doi:10.1038/nm.4407 (2017).
2 Sayin, V. I. et al. Activation of the NRF2 antioxidant program generates an imbalance in central carbon metabolism in cancer. Elife 6, doi:10.7554/eLife.28083 (2017).
3 Skoulidis, F. et al. Co-occurring genomic alterations define major subsets of KRAS-mutant lung adenocarcinoma with distinct biology, immune profiles, and therapeutic vulnerabilities. Cancer Discov 5, 860-877, doi:10.1158/2159-8290.CD-14-1236 (2015).
4 Cancer Genome Atlas Research, N. Comprehensive genomic characterization of squamous cell lung cancers. Nature 489, 519-525, doi:10.1038/nature11404 (2012).
5 Jaramillo, M. C. & Zhang, D. D. The emerging role of the Nrf2-Keap1 signaling pathway in cancer. Genes Dev 27, 2179-2191, doi:10.1101/gad.225680.113 (2013).
6 Konstantinopoulos, P. A. et al. Keap1 mutations and Nrf2 pathway activation in epithelial ovarian cancer. Cancer Res 71, 5081-5089, doi:10.1158/0008-5472.CAN-10-4668 (2011).
7 Shibata, T. et al. Genetic alteration of Keap1 confers constitutive Nrf2 activation and resistance to chemotherapy in gallbladder cancer. Gastroenterology 135, 1358-1368, 1368 e1351-1354, doi:10.1053/j.gastro.2008.06.082 (2008).
8 Kim, Y. R. et al. Oncogenic NRF2 mutations in squamous cell carcinomas of oesophagus and skin. The Journal of pathology 220, 446-451, doi:10.1002/path.2653 (2010).
9 Sato, Y. et al. Integrated molecular analysis of clear-cell renal cell carcinoma. Nat Genet 45, 860-867, doi:10.1038/ng.2699 (2013).
10 Fabrizio, F. P. et at Keap1/Nrf2 pathway in kidney cancer: frequent methylation of KEAP1 gene promoter in clear renal cell carcinoma. Oncotarget, doi:10.18632/oncotarget.14492 (2017).
11 Muscarella, L. A. et al. Regulation of KEAP1 expression by promoter methylation in malignant gliomas and association with patient's outcome. Epigenetics 6, 317-325 (2011).
12 Hanada, N. et al. Methylation of the KEAP1 gene promoter region in human colorectal cancer. BMC cancer 12, 66, doi:10.1186/1471-2407-12-66 (2012).
13 Goldstein, L. D. et al. Recurrent Loss of NFE2L2 Exon 2 Is a Mechanism for Nrf2 Pathway Activation in Human Cancers. Cell Rep 16, 2605-2617, doi:10.1016/j.celrep.2016.08.010 (2016).
14 Herbst, R. S., Heymach, J. V. & Lippman, S. M. Lung cancer. N Engl J Med 359, 1367-1380, doi:10.1056/NEJMra0802714 (2008).
15 McFadden, D. G. et al. Genetic and clonal dissection of murine small cell lung carcinoma progression by genome sequencing. Cell 156, 1298-1311, doi:10.1016/j.cell.2014.02.031 (2014).
16 Jackson, E. L. et al. The differential effects of mutant p53 alleles on advanced murine lung cancer. Cancer Res 65, 10280-10288, doi:10.1158/0008-5472.CAN-05-2193 (2005).
17 DeNicola, G. M. et al. Oncogene-induced Nrf2 transcription promotes ROS detoxification and tumorigenesis. Nature 475, 106-109, doi:nature.com/nature/journal/v475/n7354/abs/nature10189-f1.2.html#supplementary-information (2011).
18 Sayin, V. I. et al. Antioxidants accelerate lung cancer progression in mice. Sci Transl Med 6, 221ra215, doi:10.1126/scitranslmed.3007653 (2014).
19 Gorrini, C., Harris, I. S. & Mak, T. W. Modulation of oxidative stress as an anticancer strategy. Nat Rev Drug Discov 12, 931-947, doi:10.1038/nrd4002 (2013).
20 Villaruz, L. C., Kalyan, A., Zarour, H. & Socinski, M. A. Immunotherapy in lung cancer. Transl Lung Cancer Res 3, 2-14, doi:10.3978/j.issn.2218-6751.2013.10.13 (2014).
21 Brahmer, J. R. et al. Safety and activity of anti-PD-L1 antibody in patients with advanced cancer. N Engl J Med 366, 2455-2465, doi:10.1056/NEJMoa1200694 (2012).
22 Shin, D. S. & Ribas, A. The evolution of checkpoint blockade as a cancer therapy: what's here, what's next? Curr Opin Immunol 33, 23-35, doi:10.1016/j.coi.2015.01.006 (2015).
23 Dal Bello, M. G., Alama, A., Coco, S., Vanni, I. & Grossi, F. Understanding the checkpoint blockade in lung cancer immunotherapy. Drug Discov Today 22, 1266-1273, doi:10.1016/j.drudis.2017.05.016 (2017).
24 Chang, C. H. & Pearce, E. L. Emerging concepts of T cell metabolism as a target of immunotherapy. Nat Immunol 17, 364-368, doi:10.1038/ni.3415 (2016).
25 Nakaya, M. et al. Inflammatory T cell responses rely on amino acid transporter ASCT2 facilitation of glutamine uptake and mTORC1 kinase activation. Immunity 40, 692-705, doi:10.1016/j.immuni.2014.04.007 (2014).
26 Crawford, J. & Cohen, H. J. The essential role of L-glutamine in lymphocyte differentiation in vitro. J Cell Physiol 124, 275-282, doi:10.1002/jcp.1041240216 (1985).
27 Carr, E. L. et al. Glutamine uptake and metabolism are coordinately regulated by ERK/MAPK during T lymphocyte activation. J Immunol 185, 1037-1044, doi:10.4049/jimmunol.0903586 (2010).
28 Sanchez-Rivera, F. J. et al. Rapid modelling of cooperating genetic events in cancer through somatic genome editing. Nature 516, 428-431, doi:10.1038/nature13906 (2014).
29 Hassanein, M. et al. Targeting SLC1a5-mediated glutamine dependence in non-small cell lung cancer. International journal of cancer 137, 1587-1597, doi:10.1002/ijc.29535 (2015).
30 Hassanein, M. et al. SLC1A5 mediates glutamine transport required for lung cancer cell growth and survival. Clin Cancer Res 19, 560-570, doi:10.1158/1078-0432.CCR-12-2334 (2013).
31 Feig, C. et al. Targeting CXCL12 from FAP-expressing carcinoma-associated fibroblasts synergizes with anti-PD-L1 immunotherapy in pancreatic cancer. Proc Natl Acad Sci USA 110, 20212-20217, doi:10.1073/pnas.1320318110 (2013).
32 Sato, E. et al. Intraepithelial CD8+ tumor-infiltrating lymphocytes and a high CD8+/regulatory T cell ratio are associated with favorable prognosis in ovarian cancer. Proc Natl Acad Sci USA 102, 18538-18543, doi:10.1073/pnas.0509182102 (2005).
33 Romero, R. et al. Keap1 loss promotes Kras-driven lung cancer and results in dependence on glutaminolysis. Nat Med, doi:10.1038/nm.4407 (2017).
34 Wagner, M. & Wiig, H. Tumor Interstitial Fluid Formation, Characterization, and Clinical Implications. Front Oncol 5, 115, doi:10.3389/fonc.2015.00115 (2015).
35 Birsoy, K. et al. An Essential Role of the Mitochondrial Electron Transport Chain in Cell Proliferation Is to Enable Aspartate Synthesis. Cell 162, 540-551, doi:10.1016/j.cell.2015.07.016 (2015).
36 Horlbeck, M. A. et al. Compact and highly active next-generation libraries for CRISPR-mediated gene repression and activation. Elife 5, doi:10.7554/eLife.19760 (2016).
37 Konermann, S. et al. Genome-scale transcriptional activation by an engineered CRISPR-Cas9 complex. Nature 517, 583-588, doi:10.1038/nature14136 (2015).
38 Joshi, N. S. et al. Regulatory T Cells in Tumor-Associated Tertiary Lymphoid Structures Suppress Anti-tumor T Cell Responses. Immunity 43, 579-590, doi:10.1016/j.immuni.2015.08.006 (2015).

This invention may be embodied in other forms or carried out in other ways without departing from the spirit or essential characteristics thereof. Although preferred embodiments are depicted and described in detail herein, it will be apparent to those skilled in the relevant art that various modifications, additions, substitutions, and the like can be made without departing from the spirit of the invention and these are therefore considered to be within the scope of the invention as defined in the claims which follow. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A method of treating a subject having cancer, the method comprising:
selecting a subject having cancer associated with a deregulated NRF2/KEAP1 pathway; and
administering to the selected subject one or more inhibitors comprising a glutamine transporter inhibitor; a GPD2 inhibitor; or combination(s) thereof.

2. The method of claim 1, wherein the glutamine transporter inhibitor inhibits SLC1A5, SLC1A4, SLC6A19, SLC38A3, SLC38A5, SLC38A7, SLC7A5, SLC7A6, SLC7A8, SLC38A1, SLC38A2, or combinations thereof.

3. The method of claim 2, wherein the glutamine transporter inhibitor inhibits SLC1A5.

4. The method of claim 1, wherein said selecting comprises detecting deregulation of the NRF2/KEAP1 pathway in a biological sample from the subject.

5. The method of claim 1, wherein the cancer is bladder cancer, bone marrow cancer, breast cancer, cancer of the central nervous system, cervical cancer, colon cancer, endometrial cancer, cancer of the gastric system, head and neck cancer, kidney cancer, liver cancer, lung cancer, muscle cancer, ovarian cancer, pancreatic cancer, prostate cancer, skin cancer, or thyroid cancer.

6. The method of claim 5, wherein the cancer is lung cancer.

7. The method of claim 6, wherein the cancer is non-small cell lung cancer (NSCLC).

8. The method of claim 1, wherein the cancer is mediated by a KRAS gene mutation.

9. The method of claim 1, wherein the one or more inhibitors are selected from the group consisting of an antibody or binding portion thereof, a nucleic acid aptamer, a peptide inhibitor, a small molecule, and combinations thereof.

10. The method of claim 1, wherein the one or more inhibitors comprises a glutamine transporter inhibitor and/or a GPD2 inhibitor.

11. (canceled)

12. The method of claim 10, wherein the glutamine transporter inhibitor is GPNA.

13. The method of claim 1, wherein one or more inhibitors comprises a GPD2 inhibitor.

14. The method of claim 10, wherein the GPD2 inhibitor is iGP-1, iGP-5, or a combination thereof.

15. The method of claim 1, wherein the subject is a human.

16. (canceled)

17. The method of claim 1, wherein said administering one or more inhibitors occurs in combination with administration of at least one other cancer therapeutic or chemotherapeutic to the selected subject.

18. (canceled)

19. The method of claim 17, wherein the chemotherapeutic is selected from the group consisting of alkylating agents, antimetabolites, anthracyclines, antitumor antibiotics, platinum-based chemotherapeutics, and plant alkaloids.

20. The method of claim 17, wherein the chemotherapeutic is selected from the group consisting of oxaliplatin, cyclophosphamide, ifosfamide, thiotepa, melphalan, busulfan, nimustine, ranimustine, dacarbazine, procarbazine, temozolomide, cisplatin, carboplatin, nedaplatin, methotrexate, pemetrexed, fluorouracil, tegaful/uracil, doxifluridine, tegaful/gimeracil/oteracil, capecitabine, cytarabine, enocitabine, gemcitabine, 6-mercaptopurine, fuludarabin, pentostatin, cladribine, hydroxyurea, doxorubicin, epirubicin, daunorubicin, idarubicine, pirarubicin, mitoxantrone, amurubicin, actinomycin D, bleomycine, pepleomycin, mytomycin C, aclarubicin, zinostatin, vincristine, vindesine, vinblastine, vinorelbine, paclitaxel, docetaxel, irinotecan, irinotecan active metabolite (SN-38), nogitecan (topotecan), etoposide, prednisolone, dexamethasone, tamoxifen, toremifene, medroxyprogesterone, anastrozole, exemestane, letrozole, rituximab, imatinib, gefitinib, gemtuzumab ozogamicin, bortezomib, erlotinib, cetuximab, bevacizumab, sunitinib, sorafenib, dasatinib, panitumumab, asparaginase, tretinoin, arsenic trioxide, salts thereof, active metabolites thereof, and combinations thereof.

21. The method of claim 1, comprising further administering to the subject one or more immune check-point inhibitor, immune modulator, or combination(s) thereof.

22. The method of claim 21, wherein the immune check-point inhibitor is one or more therapeutic that blocks or inhibits CTLA-4, PD-1 and/or PD-L1.

23. The method of claim 21, wherein the immune modulator is selected from one or more interleukin, interferon, tumor necrosis factor (TNF) or other growth factor, colony stimulating factor, T cell modulator, cytokine or hormone which stimulate the immune response.

24. (canceled)

25. The method of claim 17, wherein said administering occurs in combination with administering to the selected subject radiation therapy, surgery, thermoablation, focused ultrasound therapy, cryotherapy, or combinations thereof.

26. The method of claim 17, wherein said administering occurs in combination with administering a glutaminase inhibitor to the selected subject.

27. The method according to claim 17, wherein said administration occurs before or simultaneously with administration of the at least one other cancer therapeutic.

28. (canceled)

29. The method according to claim 17, wherein said administration occurs after administration of the at least one other cancer therapeutic.

30. The method according to claim 17, wherein said administration improves the efficacy of the other cancer therapeutic as compared to when said administration does not occur.

31. The method of claim 1, wherein the deregulated NRF2/KEAP1 pathway results in hyperactive NRF2 signaling.

32. The method of claim 1, wherein assessing Nqo1 levels is used to detect the deregulated NRF2/KEAP1 pathway.

33. The method of claim 32, wherein the Nqo1 levels are assessed using immunohistochemical staining.

34. The method of claim 1, wherein the NRF2/KEAP1 pathway is deregulated by a loss-of-function mutation in KEAP1, a gain-of-function mutation in NRF2, loss of KEAP1 expression by copy number loss, loss of KEAP1 expression by promoter hypermethylation, increased NRF2 expression by copy number gain, or increased NRF2 expression by promoter hypomethylation.

35. The method of claim 1, wherein said administering is carried out orally, parenterally, periadventitially, subcutaneously, intravenously, intramuscularly, intraarticularly, intraperitoneally, by inhalation, by intranasal instillation, by implantation, by intracavitary or intravesical instillation, intraocularly, intraarterially, intralesionally, transdermally, or by application to mucous membranes.

36. The method of claim 1, wherein the method suppresses tumor growth or inhibits metastasis of a tumor in the selected subject.

37. (canceled)


SEQUENCE LISTING (20 sequences)

SEQ ID NO: 1 - DNA, 22 bp, Artificial Sequence
  forward primer
ctcttgcctacgccaccagctc

SEQ ID NO: 2 - DNA, 31 bp, Artificial Sequence
  reverse primer
agctagccaccatggcttgagtaagtctgca

SEQ ID NO: 3 - DNA, 23 bp, Artificial Sequence
  forward primer
gaagaaattagagggcatgcttc

SEQ ID NO: 4 - DNA, 22 bp, Artificial Sequence
  reverse primer
cttctcccagtgaccttatgta

SEQ ID NO: 5 - DNA, 23 bp, Artificial Sequence
  sgKeap1.2
gtgaacatggctttaaagactgg

SEQ ID NO: 6 - DNA, 36 bp, Artificial Sequence
  WT
cccgttggtgaacatggctttaaagactggggctgg

SEQ ID NO: 7 - DNA, 37 bp, Artificial Sequence
  Allele A
cccgttggtgaacatggcttgtaaagactggggctgg

SEQ ID NO: 8 - DNA, 35 bp, Artificial Sequence
  Allele B
cccgttggtgaacatggctttaagactggggctgg

SEQ ID NO: 9 - DNA, 25 bp, Artificial Sequence
  Allele C
cccgttggtgaacatggctttatgg

SEQ ID NO: 10 - DNA, 24 bp, Artificial Sequence
  sgKeap1.4
gtgttccacgcgtgcatcgactgg

SEQ ID NO: 11 - DNA, 35 bp, Artificial Sequence
  WT
gaggtgttccacgcgtgcatcgactgggtcaaata

SEQ ID NO: 12 - DNA, 36 bp, Artificial Sequence
  KPK1 allele
gaggtgttccacgcgtgcatccgactgggtcaaata

SEQ ID NO: 13 - DNA, 28 bp, Artificial Sequence
  KPK1 allele
gaggtgttccacgcgtgcgggtcaaata

SEQ ID NO: 14 - DNA, 31 bp, Artificial Sequence
  KPK2 allele
gaggtgttccacgcgtgcatcgggtcaaata

SEQ ID NO: 15 - DNA, 24 bp, Artificial Sequence
  sgNrf2.3
gaggttggggcagcacctgctggg

SEQ ID NO: 16 - DNA, 34 bp, Artificial Sequence
  WT
caggggcccgaggttggggcagcacctgctggga

SEQ ID NO: 17 - DNA, 33 bp, Artificial Sequence
  KPN1 allele
caggggcccgaggttggggcagcactgctggga

SEQ ID NO: 18 - DNA, 28 bp, Artificial Sequence
  KPN1 allele
caggggcccgaggttggggcagctggga

SEQ ID NO: 19 - DNA, 35 bp, Artificial Sequence
  KPN2 allele
caggggcccgaggttggggcagcaccttgctggga

SEQ ID NO: 20 - DNA, 33 bp, Artificial Sequence; identical to SEQ ID NO: 17
  KPN2 allele